(19) United States
(12) Patent Application Publication
(10) Pub. No.: US 2011/0070192 A1
(43) Pub. Date: Mar. 24, 2011

(54) Method of preparation of novel nucleoside analogs and uses

(76) Inventors: Bruno Tse, San Diego, CA (US); Borcherng Hong, Vista, CA (US); Wei-Hung Chen, Chia-Yi (TW); Yea-Fen Jiang, Chia-Yi (TW); Shuo-Cang Zhang, Chia-Yi (TW); Subramanian Marappan, San Diego, CA (US); Sepehr Sarshar, Cardiff by the Sea, CA (US)

(21) Appl. No.: 10/578,850

(22) PCT Filed: Nov. 8, 2004
(86) PCT No.: PCT/US04/37505

§ 371 Date: Apr. 6, 2007

Related U.S. Application Data
(60) Provisional application No. 60/520,462, filed on Nov. 14, 2003.

(51) Int. Cl. A61K 38/21; C07H 19/09; A61K 31/7072; C07H 21/00; C07D 403/14; A61K 31/515; A61K 31/513; C07F 7/10; A61K 31/695; C07H 19/19; A61K 31/708; C07D 473/32; A61K 31/522; A61K 31/282; A61K 33/24; A61K 31/664; A61K 31/7068; A61K 31/4985; A61P 3/10; A61P 31/00; A61P 35/00; C12N 5/071; C12Q 1/70; C12Q 1/68

(52) U.S. Cl. 424/85.4; 536/28.53; 514/50; 536/24.32; 544/314; 514/270; 514/274; 544/317; 544/229; 514/63; 536/28.4; 514/46; 544/276; 514/263.24; 514/492; 424/649; 514/110; 514/49; 514/249; 435/375; 435/5; 435/6

(57) ABSTRACT

Processes for the preparation of racemic and optically active nucleoside analogs of formula (A) are described. These compounds are useful as anti-infective agents, antisense therapeutic agents and hybridization assay probes.

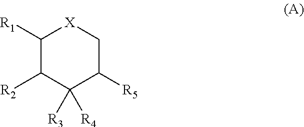

### BACKGROUND OF THE INVENTION

1. Field of the Invention

This invention relates to nucleoside analog compounds, and more particularly, to the nucleoside analogs of formula A. This invention further relates to the chemical synthesis and the pharmaceutical and/or medical use of such nucleoside analogs, including their use as anti-infectives and antisense agents.

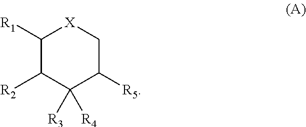

2. Background Information

In antisense therapeutic technology, synthetic oligonucleotides are introduced into a diseased cell. Antisense oligonucleotides have demonstrated effective inhibition of many viral and cellular gene products. An ideal antisense oligonucleotide has high binding affinity for the target RNA, is resistant to nuclease degradation, binds selectively to transport proteins, and is cell permeable in vivo. To achieve these goals, structural analogs of nucleic acids with modified heterocycle, sugar, and phosphodiester backbone moieties have been synthesized. Some of the most successful analogs resulted from modification of the sugar at the 2′-position.

In designing novel genetic diagnostic probes, analogs of the natural nucleosides are introduced into oligonucleotide sequences in order to increase their hybridization with complementary DNA or RNA strands. Besides enhanced hybridization, a number of other characteristics are generally recognized as desirable, including efficient oligomerization of the modified building blocks using automated DNA synthesizers, stability toward degradation by nucleases, and good aqueous solubility of oligomers. High affinity and selective recognition of complementary oligonucleotides are paramount for most applications. This property is usually estimated by comparing the melting temperature (Tₘ value) of an oligonucleotide duplex involving a partly or fully modified oligonucleotide and an unmodified complement. The Tₘ value of the corresponding unmodified duplex of complementary oligonucleotide strands then serves as standard reference.

Certain modifications enhance the aforementioned properties of non-natural oligonucleotides and others do not. For example, modification of the phosphodiester linkages with nonionic phosphoramidate or methylphosphonate linkages has shown success in increasing the thermal affinities of non-natural oligonucleotides towards complementary RNA and, especially, DNA. Introduction of conformationally restricted analogs in which the nucleoside monomers contain bi- or tricyclic carbohydrate moieties have been found to display high-affinity binding of, in particular, complementary RNA.

Infectious diseases constitute an alarming public health issue. As of the end of 2002, there were approximately 42 million people living with HIV/AIDS worldwide. It is estimated that in 2002 alone, 5 million people were infected with HIV and 3.1 million died of AIDS-related complications. Most known anti-viral agents are chemical analogs of naturally occurring nucleosides or sugars. Ganciclovir (9-[(1,3-dihydroxy-2-propoxy)methyl]guanine) is a potent inhibitor of viruses of the herpes family, including cytomegalovirus (CMV), that are pathogenic for humans and animals. The primary mechanism of ganciclovir action against CMV is inhibition of the replication of viral DNA by ganciclovir-5′-triphosphate (ganciclovir-TP). This inhibition includes a selective and potent inhibition of the viral DNA polymerase. Other examples of anti-viral therapeutics that act via similar modes of action include AZT, Acyclovir, and Valtrex™. These antiviral therapies have markedly reduced mortality rates associated with certain viral diseases. Yet, many challenges remain. First, the current therapies do not suppress viral replication in all patients and less than half of the patients actually achieve undetectable viral loads. Second, enumerable genetic mutations have given rise to drug resistant viral strains. Most importantly, viral infections remain incurable and patients suffering from such infectious diseases are in dire need of novel treatments. As such, there is an obvious and immediate need for novel nucleoside analogs that address the aforementioned topics.

### SUMMARY OF THE INVENTION

According to an embodiment of the present invention, a compound having the structure A, or a pharmaceutically acceptable salt thereof, is provided:

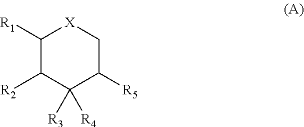

where X is oxygen, sulfur or —NR₆; R₁ is a C₁₋₁₀ a substituted alkyl or —CH₂OR₁₁; R₂ is hydrogen, halogen, —OR₁₂, —SR₁₂ or —NHR₁₂; each of R₃ and R₄ is, independently, hydrogen, halogen, azido, —CN, a C₁₋₁₀ alkylcarboxy, a C₁₋₁₀ arylcarboxy or —OSO₂R₇, with the further proviso that R₃ and R₄ cannot both be hydrogen; R₅ is a heteroaryl, saturated heterocyclic or —NR₈R₉; R₆ is hydrogen, amino protecting group, a C₁₋₁₀ alkyl, a C₁₋₁₀ substituted alkyl, an aryl, a C₁₋₁₀ alkylcarbonyl, an arylcarbonyl, a C₁₋₁₀ alkyloxycarbonyl, an aryloxycarbonyl or —SO₂R₁₀; each of R₈ and R₉ is, independently, hydrogen, or a C₁₋₁₀ alkyl, or R₈, R₉ and the nitrogen atom to which R₈ and R₉ are attached, combine to form a saturated heterocyclic or heteroaryl ring; each of R₇ and R₁₀ is, independently, a C₁₋₁₀ alkyl, a C₁₋₁₀ substituted alkyl, an aryl or a substituted aryl; R₁₁ is hydrogen, a hydroxyl protecting group, —P(O)(OR₁₅)(OR₁₆), or —CH₂P(O)(OR₁₅)(OR₁₆); R₁₂ is hydrogen, —PR₁₃R₁₄, a hydroxyl protecting group if R₂ is —OR₁₂, a thiol protecting group if R₂ is —SR₁₂, or an amino protecting group if R₂ is —NHR₁₂; or if R₁ is —CH₂OR₁₁ and R₂ is —OR₁₂, then R₁₁, R₁₂ and the oxygen atoms to which R₁₁ and R₁₂ are attached, combine to form a cyclic acetal or ketal; each of R₁₃ and R₁₄ is, independently, —NR₈R₉ or —OCH₂CH₂CN; and each of R₁₅ and R₁₆ is, independently, hydrogen or an C₁₋₁₀ alkyl.

Some examples of compounds that can be used, or pharmaceutically acceptable salts thereof, include the compounds having the formulae:

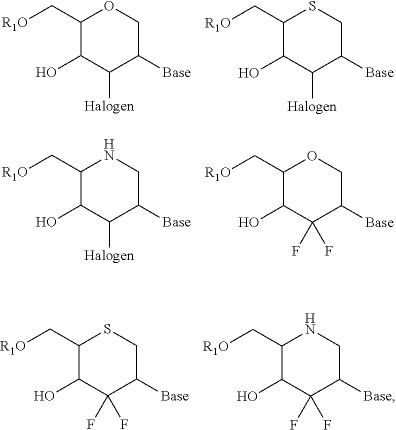

where R₁ is —H, PO₃H or —CH₂OPO₃H; the halogen F, Cl, Br or I; and the base is a moiety selected from the group having the formulae:

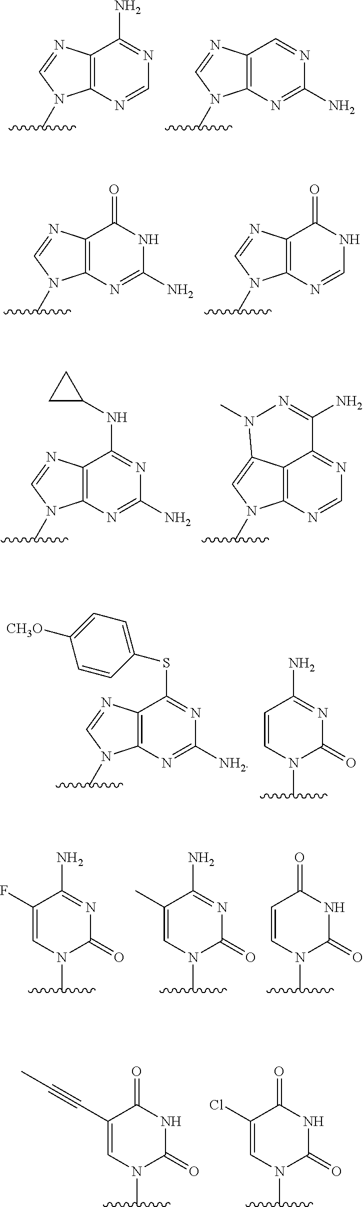

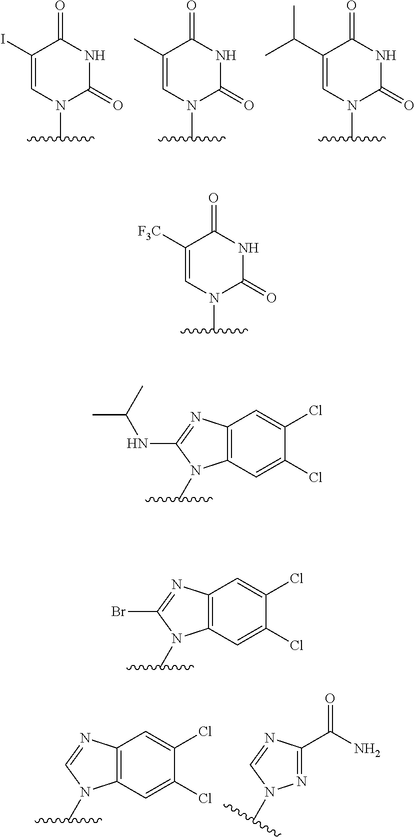

According to another embodiment of the present invention, a pharmaceutical composition is provided, the composition comprising at least one of any compound described above, and pharmaceutically acceptable pro-drugs and salts thereof. The pharmaceutical composition can further include a pharmaceutically acceptable vehicle, for enteral, parenteral, topical or ocular administration, and can be used for the treatment or prophylaxis of various infections, e.g., viral infections, bacterial infections, fungal infections. The pharmaceutical composition can be also used for the treatment or prophylaxis of cancer, diabetes and other diseases of genetic origin. The pharmaceutical composition can be also used in antisense therapy, including for the treatment or prophylaxis of cancer, diabetes and other diseases of genetic origin.

According to yet another embodiment of the present invention, a method for treating cancer, is provided, the method comprising administering to a subject in need thereof an effective amount of at least one compound described above, in a pharmaceutically acceptable vehicle. Examples of types of cancer that can be treated include mammary cancer, prostate cancer, kidney cancer, Karposi's sarcoma, colon cancer, cervical cancer, lung cancer, cutaneous T-cell lymphoma, cancer of the head and neck, cancers of the aerodigestive pathway, skin cancer, bladder cancer, sarcomas, leukoplakias, and acute promyelocytic leukemia. To treat cancer, the method can be used in combination with using at least one other chemotherapeutic agent, such as Busulfan, Carboplatin, Cisplatin, Cyclophosphamide, Cytosine arabinoside, Etoposide, 5-Fluorouracil, Melphalan, Methotrexate, Mitoxantrone, Taxol, Interferon, Fareston, Arzoxifene, Evista, and Tamoxifen.

According to another embodiment of the present invention, a method for modulating the expression of enzymes, proteins, nuclear factors or receptors in cells or tissues is provided, the method comprising contacting the cells or tissues with at least one compound or composition described above.

According to yet another embodiment of the present invention, a nucleic acid probe is provided, the probe being constructed from at least one compound described above. The nucleic acid probe can be used, for example, for the identification and quantification of a bacterium, virus or any other organism in sputum, urine, blood, tissue sections, food, soil, or water.

### DETAILED DESCRIPTION

### Definitions

The compounds according to this invention may contain one or more asymmetric carbon atoms and can thus occur as racemates and racemic mixtures, single enantiomers, diastereomeric mixtures or individual diastereomers. The term “stereoisomer” refers to a chemical compound having the same molecular weight, chemical composition, and constitution as another, but with the atoms grouped differently. That is, certain identical chemical moieties are at different orientations in space and, therefore, when pure, have the ability to rotate the plane of polarized light. However, some pure stereoisomers may have an optical rotation that is so slight that it is undetectable with present instrumentation. The compounds described herein may have one or more asymmetrical carbon atoms and therefore include various stereoisomers. All such isomeric forms of these compounds are expressly included in the present invention.

Each stereogenic carbon may be of R or S configuration. Although the specific compounds exemplified in this application may be depicted in a particular configuration, compounds having either the opposite stereochemistry at any given chiral center or mixtures thereof are also envisioned. When chiral centers are found in the derivatives of this invention, it is to be understood that this invention encompasses all possible stereoisomers.

The terms “optically pure compound” or “optically pure isomer” refers to a single stereoisomer of a chiral compound regardless of the configuration of the said compound.

For the purpose of this application, all sugars are referenced using conventional three-letter nomenclature. All sugars are assumed to be in the D-form unless otherwise noted, except for fucose, which is in the L-form. Further, all sugars are in the pyranose form.

The following example of nomenclature and numbering system is provided for reference.

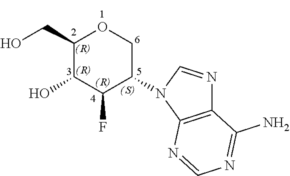

### (2R,3R,4R,5S)-5-(6-Amino-purin-9-yl)-4-fluoro-2-hydroxymethyl-tetrahydro-pyran-3-ol

The term “substantially homogeneous” refers to collections of molecules wherein at least 80%, preferably at least about 90% and more preferably at least about 95% of the molecules are a single compound or a single stereoisomer thereof.

As used herein, the term “attached” signifies a stable covalent bond, certain preferred points of attachment being apparent to those skilled in the art.

The terms “optional” or “optionally” refer to occurrence or non-occurence of the subsequently described event or circumstance, and that the description includes instances where said event or circumstance occurs and instances where it does not. In such context, the sentence “optionally substituted alkyl group” means that the alkyl group may or may not be substituted and the description includes both a substituted and an unsubstituted alkyl group.

The term “effective amount” of a compound refers a non-toxic but sufficient amount of the compound that provides a desired effect. This amount may vary from subject to subject, depending on the species, age, and physical condition of the subject, the severity of the disease that is being treated, the particular compound used, its mode of administration, and the like. Therefore, it is difficult to generalize an exact “effective amount,” yet, a suitable effective amount may be determined by one of ordinary skill in the art.

The term “pharmaceutically acceptable” refers to a compound, additive or composition that is not biologically or otherwise undesirable. For example, the additive or composition may be administered to a subject along with a compound of the invention without causing any undesirable biological effects or interacting in an undesirable manner with any of the other components of the pharmaceutical composition in which it is contained.

The term “pharmaceutically acceptable salts” includes hydrochloric salt, hydrobromic salt, hydroiodic salt, hydrofluoric salt, sulfuric salt, citric salt, maleic salt, acetic salt, lactic salt, nicotinic salt, succinic salt, oxalic salt, phosphoric salt, malonic salt, salicylic salt, phenylacetic salt, stearic salt, pyridine salt, ammonium salt, piperazine salt, diethylamine salt, nicotinamide salt, formic salt, urea salt, sodium salt, potassium salt, calcium salt, magnesium salt, zinc salt, lithium salt, cinnamic salt, methylamino salt, methanesulfonic salt, picric salt, tartaric salt, triethylamino salt, dimethylamino salt, tris(hydroxymethyl)aminomethane salt and the like. Additional pharmaceutically acceptable salts are known to those of skill in the art.

When used in conjunction with a compound of this invention, the terms “elicite”, “eliciting,” modulator”, “modulate”, “modulating”, “regulator”, “regulate” or “regulating” selective gene expression refer to a compound that can act as an activator, an agonist, a pan-agonist or an antagonist of gene expression by a particular receptor, such as for example a Retinoid X Receptor and the like.

The terms “therapeutic agent” and “chemotherapeutic agent”, refer to a compound or compounds and pharmaceutically acceptable compositions thereof that are administered to mammalian subjects as prophylactic or remedy in the treatment of a disease or medical condition. Such compounds may be administered to the subject via oral formulation, transdermal formulation or by injection.

The term “Lewis acid” refers to a molecule that can accept an unshared pair of electrons and as such would be obvious to one of ordinary skill and knowledge in the art. The definition of “Lewis acid” includes but is not limited to: boron trifluoride, boron trifluoride etherate, boron trifluoride tetrahydrofuran complex, boron trifluoride tert-butyl-methyl ether complex, boron trifluoride dibutyl ether complex, boron trifluoride dihydrate, boron trifluoride di-acetic acid complex, boron trifluoride dimethyl sulfide complex, boron trichloride, boron trichloride dimethyl sulfide complex, boron tribromide, boron tribromide dimethyl sulfide complex, boron triiodide, triimethoxyborane, triethoxyborane, trimethylaluminum, triethylaluminum, aluminum trichloride, aluminum trichloride tetrahydrofuran complex, aluminum tribromide, titanium tetrachloride, titanium tetrabromide, titanium iodide, titanium tetraethoxide, titanium tetraisopropoxide, scandium (III) trifluoromethanesulfonate, yttrium (III) trifluoromethanesulfonate, ytterbium (III) trifluoromethanesulfonate, lanthanum (III) trifluoromethanesulfonate, zinc (II) chloride, zinc (II) bromide, zinc (II) iodide, zinc (II) trifluoromethanesulfonate, zinc (II) sulfate, magnesium sulfate, lithium perchlorate, copper (II) trifluoromethanesulfonate, copper (II) tetrafluoroborate and the like. Certain Lewis acids may have optically pure ligands attached to the electron acceptor atom, as set forth in Corey, E. J. Angewandte Chemie, International Edition (2002), 41(10), 1650-1667; Aspinall, H. C. Chemical Reviews (Washington, D.C., United States) (2002), 102(6), 1807-1850; Groger, H. Chemistry—A European Journal (2001), 7(24), 5246-5251; Davies, H. M. L. Chemtracts (2001), 14(11), 642-645; Wan, Y. Chemtracts (2001), 14(11), 610-615; Kim, Y. H. Accounts of Chemical Research (2001), 34(12), 955-962; Seebach, D. Angewandte Chemie, International Edition (2001), 40(1), 92-138; Blaser, H. U. Applied Catalysis, A: General (2001), 221(1-2), 119-143; Yet, L. Angewandte Chemie, International Edition (2001), 40(5), 875-877; Jorgensen, K. A. Angewandte Chemie, International Edition (2000), 39(20), 3558-3588; Dias, L. C. Current Organic Chemistry (2000), 4(3), 305-342; Spindler, F. Enantiomer (1999), 4(6), 557-568; Fodor, K. Enantiomer (1999), 4(6), 497-511; Shimizu, K. D.; Comprehensive Asymmetric Catalysis (1999), 3, 1389-1399; Kagan, H. B. Comprehensive Asymmetric Catalysis (1999), 1, 9-30; Mikami, K. Lewis Acid Reagents (1999), 93-136 and all references cited therein. Such Lewis acids may be used by one of ordinary skill and knowledge in the art to produce optically pure compounds from achiral starting materials.

The term “acylating agent” refers to a molecule that can transfer an alkylcarbonyl, substituted alkylcarbonyl or aryl carbonyl group to another molecule. The definition of “acylating agent” includes but is not limited to ethyl acetate, vinyl acetate, vinyl propionate, vinyl butyrate, isopropenyl acetate, 1-ethoxyvinyl acetate, trichloroethyl butyrate, trifluoroethyl butyrate, trifluoroethyl laureate, S-ethyl thiooctanoate, biacetyl monooxime acetate, acetic anhydride, acetyl chloride, succinic anhydride, diketene, diallyl carbonate, carbonic acid but-3-enyl ester cyanomethyl ester, amino acid and the like.

The term “nucleophile” or “nucleophilic reagent” refers to a negatively charged or neutral molecule that has an unshared pair of electrons and as such would be obvious to one of ordinary skill and knowledge in the art. The definition of “nucleophile” includes but is not limited to: water, alkylhydroxy, alkoxy anion, arylhydroxy, aryloxy anion, alkylthiol, alkylthio anion, arylthiol, arylthio anion, ammonia, alkylamine, arylamine, alkylamine anion, arylamine anion, hydrazine, alkyl hydrazine, arylhydrazine, alkylcarbonyl hydrazine, arylcarbonyl hydrazine, hydrazine anion, alkyl hydrazine anion, arylhydrazine anion, alkylcarbonyl hydrazine anion, arylcarbonyl hydrazine anion, cyanide, azide, hydride, alkyl anion, aryl anion and the like.

The term “electrophile” or “electrophilic reagent” refers to a positively charged or neutral molecule that has an open valence shell and as such would be obvious to one of ordinary skill and knowledge in the art. The definition of “electrophile” includes but is not limited to: hydronium, acylium, lewis acids, such as for example, boron trifluoride and the like, halogens, such as for example Br₂ and the like, carbocations, such as for example tert-butyl cation and the like, diazomethane, trimethylsilyldiazomethane, alkyl halides, such as for example methyl iodide, benzyl bromide and the like, alkyl triflates, such as for example methyl triflate and the like, alkyl sulfonates, such as for example ethyl toluenesulfonate, butyl methanesulfonate and the like, acyl halides, such as for example acetyl chloride, benzoyl bromide and the like, acid anhydrides, such as for example acetic anhydride, succinic anhydride, maleic anhydride and the like, isocyanates, such as for example methyl isocyanate, phenylisocyanate and the like, chloroformates, such as for example methyl chloroformate, ethyl chloroformate, benzyl chloroformate and the like, sulfonyl halides, such as for example methanesulfonyl chloride, p-toluenesulfonyl chloride and the like, silyl halides, such as for example trimethylsilyl chloride, tertbutyldimethyl silyll chloride and the like, phosphoryl halide such as for example dimethyl chlorophosphate and the like, alpha-beta-unsaturated carbonyl compounds such as for example acrolein, methyl vinyl ketone, cinnamaldehyde and the like.

The term “leaving group” refers to any atom (or group of atoms) that is stable in its anion or neutral form after it has been displaced by a nucleophile and as such would be obvious to one of ordinary skill and knowledge in the art. The definition of “leaving group” includes but is not limited to: water, methanol, ethanol, chloride, bromide, iodide, methanesulfonate, tolylsulfonate, trifluoromethanesulfonate, acetate, trichloroacetate, benzoate and the like.

The term “oxidant” refers to any reagent that will increase the oxidation state of a carbon atom in the starting material by either adding an oxygen atom to this carbon or removing an electron from this carbon and as such would be obvious to one of ordinary skill and knowledge in the art. The definition of “oxidant” includes but is not limited to: osmium tetroxide, ruthenium tetroxide, ruthenium trichloride, potassium permanganate, meta-chloroperbenzoic acid, hydrogen peroxide, dimethyl dioxirane and the like.

The term “metal ligand” refers to a molecule that has an unshared pair of electrons and can coordinate to a metal atom and as such would be obvious to one of ordinary skill and knowledge in the art. The definition of “metal ligand” includes but is not limited to: water, alkoxy anion, alkylthio anion, ammonia, trialkylamine, triarylamine, trialkylphosphine, triarylphosphine, cyanide, azide and the like.

The term “reducing reagent” refers to any reagent that will decrease the oxidation state of a carbon atom in the starting material by either adding a hydrogen atom to this carbon or adding an electron to this carbon and as such would be obvious to one of ordinary skill and knowledge in the art. The definition of “reducing reagent” includes but is not limited to: borane-dimethyl sulfide complex, 9-borabicyclo[3.3.1]nonane (9-BBN), catechol borane, lithium borohydride, sodium borohydride, sodium borohydride-methanol complex, potassium borohydride, sodium hydroxyborohydride, lithium triethylborohydride, lithium n-butylborohydride, sodium cyanoborohydride, calcium (II) borohydride, lithium aluminum hydride, diisobutylaluminum hydride, n-butyl-diisobutylaluminum hydride, sodium bis-methoxyethoxyaluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The term “coupling reagent” refers to any reagent that will activate the carbonyl of a carboxylic acid and facilitate the formation of an ester or amide bond. The definition of “coupling reagent” includes but is not limited to: acetyl chloride, ethyl chloroformate, dicyclohexylcarbodiimide (DCC), diisopropyl carbodiiimide (DIC), 1-ethyl-3-(3-dimethylaminopropyl) carbodiimide (EDCI), N-hydroxybenzotriazole (HOBT), N-hydroxysuccinimide (HOSu), 4-nitrophenol, pentafluorophenol, 2-(1H-benzotriazole-1-yl)-1,1,3,3-tetramethyluronium tetrafluoroborate (TBTU), O-benzotriazole-N,N,N′N′-tetramethyluronium hexafluorophosphate (HBTU), benzotriazole-1-yl-oxy-tris-(dimethylamino)-phosphonium hexafluorophosphate (BOP), benzotriazole-1-yl-oxy-tris-pyrrolidinophosphonium hexafluorophosphate, bromo-trispyrrolidino-phosphonium hexafluorophosphate, 2-(5-norbornene-2,3-dicarboximido)-1,1,3,3-tetramethyluronium tetrafluoroborate (TNTU), O—(N-succinimidyl)-1,1,3,3-tetramethyluronium tetrafluoroborate (TSTU), tetramethylfluoroformamidinium hexafluorophosphate and the like.

The term “removable protecting group” or “protecting group” refers to any group which when bound to a functionality, such as the oxygen atom of a hydroxyl or carboxyl group or the nitrogen atom of an amino group, prevents reactions from occurring at these functional groups and which protecting group can be removed by conventional chemical or enzymatic steps to reestablish the functional group. The particular removable protecting group employed is not critical.

The definition of “hydroxyl protecting group” includes but is not limited to:
a) Methyl, tert-butyl, allyl, propargyl, p-chlorophenyl, p-methoxyphenyl, p-nitrophenyl, 2,4-dinitrophenyl, 2,3,5,6-tetrafluoro-4-(trifluoromethyl)phenyl, methoxymethyl, methylthiomethyl, (phenyldimethylsilyl)methoxymethyl, benzyloxymethyl, p-methoxy-benzyloxymethyl, p-nitrobenzyloxymethyl, o-nitrobenzyloxymethyl, (4-methoxyphenoxy)methyl, guaiacolmethyl, tert-butoxymethyl, 4-pentenyloxymethyl, tert-butyldimethylsiloxymethyl, thexyldimethylsiloxymethyl, tert-butyldiphenylsiloxymethyl, 2-methoxyethoxymethyl, 2,2,2-trichloroethoxymethyl, bis(2-chloroethoxy)methyl, 2-(trimethylsilypethoxymethyl, menthoxymethyl, 1-ethoxyethyl, 1-(2-chloroethoxy)ethyl, 1-[2-(trimethylsilyl)ethoxy]ethyl, 1-methyl-1-ethoxyethyl, 1-methyl-1-benzyloxyethyl, 1-methyl-1-benzyloxy-2-fluoroethyl, 1-methyl-1-phenoxyethyl, 2,2,2-trichloroethyl, 1-dianisyl-2,2,2-trichloroethyl, 1,1,1,3,3,3-hexafluoro-2-phenylisopropyl, 2-trimethylsilylethyl, 2-(benzylthio)ethyl, 2-(phenylselenyl)ethyl, tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydropyranyl S,S-dioxide, 1-[(2-chloro-4-methyl)phenyl]-4-methoxypiperidin-4-yl, 1-(2-fluorophenyl)-4-methoxypiperidin-4-yl, 1,4-dioxan-2-yl, tetrahydrofuranyl, tetrahydrothiofuranyl and the like;
b) Benzyl, 2-nitrobenzyl, 2-trifluoromethylbenzyl, 4-methoxybenzyl, 4-nitrobenzyl, 4-chlorobenzyl, 4-bromobenzyl, 4-cyanobenzyl, 4-phenylbenzyl, 4-acylaminobenzyl, 4-azidobenzyl, 4-(methylsulfinyl)benzyl, 2,4-dimethoxybenzyl, 4-azido-3-chlorobenzyl, 3,4-dimethoxybenzyl, 2,6-dichlorobenzyl, 2,6-difluorobenzyl, 1-pyrenylmethyl, diphenylmethyl, 4,4′-dinitrobenzhydryl, 5-benzosuberyl, triphenylmethyl (Trityl), □-naphthyldiphenylmethyl, (4-Methoxyphenyl)-diphenyl-methyl, di-(p-methoxyphenyl)-phenylmethyl, tri-(p-methoxyphenyl)methyl, 4-(4′-bromophenacyloxy)-phenyldiphenylmethyl, 4,4′,4″-tris(4,5-dichlorophthalimidophenyl)methyl, 4,4′,4″-tris(levulinoyloxyphenyl)methyl, 4,4′-dimethoxy-3″-[N-(imidazolylmethyl)]trityl, 4,4′-dimethoxy-3″-[N-(imidazolylethyl)carbamoyl]trityl, 1,1-bis(4-methoxyphenyl)-1′-pyrenylmethyl, 4-(17-tetrabenzo[a,c,g,I]fluorenylmethyl)-4,4′-dimethoxytrityl, 9-anthryl, 9-(9-phenyl)xanthenyl, 9-(9-phenyl-10-oxo)anthryl and the like;
c) Trimethylsilyl, triethylsilyl, triisopropylsilyl, dimethylisopropylsilyl, diethylisopropylsilyl, dimethylhexylsilyl, tert-butyldimethylsilyl, tert-butyldiphenylsilyl, tribenzylsilyl, tri-p-xylylsilyl, triphenylsilyl, diphenylmethylsilyl, di-tert-butylmethylsilyl, tris(trimethylsilyl)silyl, (2-hydroxystyryl)dimethylsilyl, (2-hydroxystyryl)diisopropylsilyl, tert-butylmethoxyphenylsilyl, tert-butoxydiphenylsilyl and the like;
d) —C(O)R₂₀, where R₂₀ is selected from alkyl, substituted alkyl, aryl and more specifically R₂₀=hydrogen, methyl, ethyl, tert-butyl, adamantyl, crotyl, chloromethyl, dichloromethyl, trichloromethyl, trifluoromethyl, methoxymethyl, triphenylmethoxymethyl, phenoxymethyl, 4-chlorophenoxymethyl, phenylmethyl, diphenylmethyl, 4-methoxycrotyl, 3-phenylpropyl, 4-pentenyl, 4-oxopentyl, 4,4-(ethylenedithio)pentyl, 5-[3-bis(4-methoxyphenyl)hydroxymethylphenoxy]-4-oxopentyl, phenyl, 4-methylphenyl, 4-nitrophenyl, 4-fluorophenyl, 4-chlorophenyl, 4-methoxyphenyl, 4-phenylphenyl, 2,4,6-trimethylphenyl, □-naphthyl, benzoyl and the like;
e) —C(O)OR₂₀, where R₂₀ is selected from alkyl, substituted alkyl, aryl and more specifically R₂₀=methyl, methoxymethyl, 9-fluorenylmethyl, ethyl, 2,2,2-trichloromethyl, 1,1-dimethyl-2,2,2-trichloroethyl, 2-(trimethylsilyl)ethyl, 2-(phenylsulfonyl)ethyl, isobutyl, tert-Butyl, vinyl, allyl, 4-nitrophenyl, benzyl, 2-nitrobenzyl, 4-nitrobenzyl, 4-methoxybenzyl, 2,4-dimethoxybenzyl, 3,4-dimethoxybenzyl, 2-(methylthiomethoxy)ethyl, 2-dansenylethyl, 2-(4-nitrophenyl)ethyl, 2-(2,4-dinitrophenyl)ethyl, 2-cyano-1-phenylethyl, thiobenzyl, 4-ethoxy-1-naphthyl and the like.

The definition of “amino protecting group” includes but is not limited to:
a) 2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 1-methyl-1-(triphenylphosphonio)ethyl, 1,1-dimethyl-2-cyanoethyl, 2-dansylethyl, 2-(4-nitrophenyl)ethyl, 4-phenylacetoxybenzyl, 4-azidobenzyl, 4-azidomethoxybenzyl, m-chloro-p-acyloxybenzyl, p-(dihydroxyboryl)benzyl, 5-benzisoxazolylmethyl, 2-(trifluoromethyl)-6-chromonylmethyl, m-nitrophenyl, 3,5-dimethoxybenzyl, 1-methyl-1-(3,5-dimethoxyphenyl)ethyl, o-nitrobenzyl, □-methylnitropiperonyl, 3,4-dimethoxy-6-nitrobenzyl, N-benzenesulfenyl, N-o-nitrobenzenesulfenyl, N-2,4-dinitrobenzenesulfenyl, N-pentachlorobenzenesulfenyl. N-2-nitro-4-methoxybenzenesulfenyl, N-triphenylmethylsulfenyl, N-1-(2,2,2-trifluoro-1,1-diphenyl)ethylsulfenyl, N-3-nitro-2-pyridinesulfenyl, N-p-toluenesulfonyl, N-benzenesulfonyl, N-2,3,6-trimethyl-4-methoxybenzenesulfonyl, N-2,4,6-trimethoxybenzene-sulfonyl, N-2,6-dimethyl-4-methoxybenzenesulfonyl, N-pentamethylbenzenesulfonyl, N-2,3,5,6-tetramethyl-4-methoxybenzenesulfonyl and the like;
b) —C(O)OR₂₀, where R₂₀ is selected from alkyl, substituted alkyl, aryl and more specifically R₂₀=methyl, ethyl, 9-fluorenylmethyl, 9-(2-sulfo)fluorenylmethyl. 9-(2,7-dibromo)fluorenylmethyl, 17-tetrabenzo[a,c,g,i]fluorenylmethyl. 2-chloro-3-indenylmethyl, benz[f]inden-3-ylmethyl, 2,7-di-t-butyl-[9-(10,10-dioxo-10,10,10,10-tetrahydrothloxanthyl)]methyl, 1,1-dioxobenzo[b]thiophene-2-ylmethyl, 2,2,2-trichloroethyl, 2-trimethylsilylethyl, 2-phenylethyl, 1-(1-adamantyl)-1-methylethyl, 2-chloroethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-tert-butylphenyl)-1-methylethyl, 2-(2′-pyridyl)ethyl, 2-(4′-pyridyl)ethyl, 2,2-bis(4′-nitrophenyl)ethyl, N-(2-pivaloylamino)-1,1-dimethylethyl, 2-[(2-nitrophenyl)dithio]-1-phenylethyl, tert-butyl, 1-adamantyl, 2-adamantyl, Vinyl, allyl, 1-Isopropylallyl, cinnamyl 4-nitrocinnamyl, 3-(3′-pyridyl)prop-2-enyl, 8-quinolyl, N-Hydroxypiperidinyl, alkyldithio, benzyl, p-methoxybenzyl, p-nitrobenzyl, p-bromobenzyl. p-chlorobenzyl, 2,4-dichlorobenzyl, 4-methylsulfinylbenzyl, 9-anthrylmethyl, diphenylmethyl, tert-amyl, S-benzyl thiocarbamate, butynyl, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N′-dimethylcarboxamido)benzyl, 1,1-dimethyl-3-(N,N′-dimethylcarboxamido)propyl, 1,1-dimethylpropynyl, di(2-pyridyl)methyl, 2-furanylmethyl, 2-Iodoethyl, isobornyl, isobutyl, isonicotinyl, p-(p′-methoxyphenylazo)benzyl, 1-methylcyclobutyl, 1-methylcyclohexyl, 1-methyl-1-cyclopropylmethyl, 1-methyl-1-(p-phenylazophenyl)ethyl, 1-methyl-1-phenylethyl, 1-methyl-1-4′-pyridylethyl, phenyl, p-(phenylazo)benzyl, 2,4,6-tri-methylphenyl, 4-(trimethylammonium)benzyl, 2,4,6-trimethylbenzyl and the like.

The definition of “carboxyl protecting group” includes but is not limited to: 2-N-(morpholino)ethyl, choline, methyl, methoxyethyl, 9-Fluorenylmethyl, methoxymethyl, methylthiomethyl, tetrahydropyranyl, tetrahydrofuranyl, methoxyethoxymethyl, 2-(trimethylsilyl)ethoxymethyl, benzyloxymethyl, pivaloyloxymethyl, phenylacetoxymethyl, triisopropylsilylmethyl, cyanomethyl, acetol, p-bromophenacyl. □-methylphenacyI, p-methoxyphenacyl, desyl, carboxamidomethyl, p-azobenzenecarboxamido-methyl, N-phthalimidomethyl, (methoxyethoxy)ethyl, 2,2,2-trichloroethyl, 2-fluoroethyl, 2-chloroethyl, 2-bromoethyl, 2-iodoethyl, 4-chlorobutyl, 5-chloropentyl, 2-(trimethylsilyl)ethyl, 2-methylthioethyl, 1,3-dithianyl-2-methyl, 2-(p-nitrophenylsulfenyl)ethyl, 2-(p-toluenesulfonyl)ethyl, 2-(2′-pyridyl)ethyl, 2-(p-methoxyphenyl)ethyl, 2-(diphenylphosphino)ethyl, 1-methyl-1-phenylethyl, 2-(4-acetyl-2-nitrophenyl)ethyl, 2-cyanoethyl, heptyl, tert-butyl, 3-methyl-3-pentyl, dicyclopropylmethyl, 2,4-dimethyl-3-pentyl, cyclopentyl, cyclohexyl, allyl, methallyl, 2-methylbut-3-en-2-yl, 3-methylbut-2-(prenyl), 3-buten-1-yl, 4-(trimethylsilyl)-2-buten-1-yl, cinnamyI, □-methylcinnamyl, propargyl, phenyl, 2,6-dimethylphenyl, 2,6-diisopropylphenyl, 2,6-di-tert-butyl-4-methylphenyl, 2,6-di-tert-butyl-4-methoxyphenyl, p-(methylthio)phenyl, pentafluorophenyl, benzyl, triphenylmethyl, diphenylmethyl, bis(o-nitrophenyl)methyl, 9-anthrylmethyl, 2-(9,10-dioxo)anthrylmethyl. 5-dibenzosuberyl, 1-pyrenylmethyl, 2-(trifluoromethyl)-6-chromonylmethyl, 2,4,6-trimethylbenzyl, p-bromobenzyl, o-nitrobenzyl, p-nitrobenzyl, p-methoxybenzyl, 2,6-dimethoxybenzyl, 4-(methylsulfinyl)benzyl, 4-Sulfobenzyl, 4-azidomethoxybenzyl, 4-{a/-[1-(4,4-dimethyl-2,6-dioxocyclohexylidene)-3-methylbutyl]amino}benzyl, piperonyl, 4-picolyl, trimethylsilyl, triethylsilyl, tert-butyldimethylsilyl, isopropyldimethylsilyl, phenyldimethylsilyl, di-tert-butylmethylsilyl, triisopropylsilyl and the like.

The definition of “thiol protecting group” includes but is not limited to:
a) Alkyl, benzyl, 4-methoxybenzyl, 2-hydroxybenzyl, 4-hydroxybenzyl, 2-acetoxybenzyl, 4-acetoxybenzyl, 4-nitrobenzyl, 2,4,6-trimethylbenzyl, 2,4,6-trimethoxybenzyl, 4-picolyl, 2-quinolinylmethyl, 2-picolyl n-oxido, 9-anthrylmethyl, 9-fluorenylmethyl, xanthenyl, ferrocenylmethyl and the like;
b) Diphenylmethyl, bis(4-methoxyphenyl)methyl, 5-dibenzosuberyl, triphenylmethyl, diphenyl-4-pyridylmethyl, phenyl, 2,4-dinitrophenyl, tert-butyl, 1-adamantyl and the like;
c) Methoxymethyl, isobutoxymethyl, benzyloxymethyl, 2-tetrahydropyranyl, benzylthiomethyl, phenylthiomethyl, acetamidomethyl, trimethylacetamidomethyl, benzamidomethyl, allyl oxycarbonylaminomethyl, phenylacetamidomethyl, phthalimidomethyl, acetyl, carboxy-, cyanomethyl and the like;
d) (2-nitro-1-phenyl)ethyl, 2-(2,4-dinitrophenyl)ethyl, 2-(4′-pyridyl)ethyl, 2-cyanoethyl, 2-(trimethylsilyl)ethyl, 2,2-bis(carboethoxy)ethyl, 1-(3-nitrophenyl)-2-benzoyl-ethyl, 2-phenylsulfonylethyl, 1-(4-methylphenylsulfonyl)-2-methylpro4-2-yl and the like;
e) Trimethylsilyl, triethylsilyl, triisopropylsilyl, dimethylisopropylsilyl, diethylisopropylsilyl, dimethylhexylsilyl, tert-butyldimethylsilyl, tert-butyldiphenylsilyl, tribenzylsilyl, tri-p-xylylsilyl, triphenylsilyl, diphenylmethylsilyl, di-tert-butylmethylsilyl, tris(trimethylsilyl)silyl, (2-hydroxystyryl)dimethylsilyl, (2-hydroxystyryl)diisopropylsilyl, tert-butylmethoxyphenylsilyl, tert-butoxydiphenylsilyl and the like;
f) Benzoyl, trifluoroacetyl, N-[[(4-biphenylyl)isopropoxy]carbonyl]-N-methyl-□-aminothiobutyrate, N-(t-butoxycarbonyl)-N-methyl-γ-aminothiobutyrate and the like;
g) 2,2,2-Trichloroethoxycarbonyl, tert-butoxycarbonyl, benzyloxycarbonyl, 4-methoxybenzyloxycarbonyl and the like;
h) N-(Ethylamino)carbonyl, N-(methoxymethylamino)carbonyl and the like;
i) Ethylthio, tert-butylthio, phenylthio, substituted phenylthio and the like;
j) (Dimethylphosphino)thioyl, (diphenylphosphino)thioyl and the like;
k) Sulfonate, alkyloxycarbonylthio, benzyloxycarbonylthio, 3-nitro-2-pyridinethio and the like;
l) Tricarbonyl[1,2,3,4,5-η]-2,4-cyclohexadien-1-yl]-iron(1+) and the like.

The term “Amino acid” refers to any of the naturally occurring amino acids, as well as synthetic analogs and derivatives thereof. Alpha-Amino acids comprise a carbon atom to which is bonded an amino group, a carboxy group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine), substituted arylalkyl (e.g., as in tyrosine), heteroarylalkyl (e.g., as in tryptophan, histidine) and the like. One of skill in the art will appreciate that the term “amino acid” can also include beta-, gamma-, delta-, omega-amino acids, and the like. Unnatural amino acids are also known in the art, as set forth in, Natchus, M. G. Organic Synthesis: Theory and Applications (2001), 5, 89-196; Ager, D. J. Current Opinion in Drug Discovery & Development (2001), 4(6), 800; Reginato, G. Recent Research Developments in Organic Chemistry (2000), 4(Pt. 1), 351-359; Dougherty, D. A. Current Opinion in Chemical Biology (2000), 4(6), 645-652; Lesley, S. A. Drugs and the Pharmaceutical Sciences (2000), 101(Peptide and Protein Drug Analysis), 191-205; Pojitkov, A. E. Journal of Molecular Catalysis B: Enzymatic (2000), 10(1-3), 47-55; Ager, D. J. Speciality Chemicals (1999), 19(1), 10-12, and all references cited therein. Stereoisomers (e.g., D-amino acids) of the twenty conventional amino acids, unnatural amino acids such as alpha, alpha-disubstituted amino acids and other unconventional amino acids may also be suitable components for compounds of the present invention. Examples of unconventional amino acids include: 4-hydroxyproline, 3-methylhistidine, 5-hydroxylysine, and other similar amino acids and imino acids (e.g., 4-hydroxyproline).

The term “N-protected amino acid” refers to any amino acid which has a protecting group bound to the nitrogen of the amino functionality. This protecting group prevents reactions from occurring at the amino functional group and can be removed by conventional chemical or enzymatic steps to reestablish the amino functional group. The particular protecting group employed is not critical.

The term “O-protected amino acid” refers to any amino acid which has a protecting group bound to the oxygen of the carboxyl functionality. This protecting group prevents reactions from occurring at the carboxyl functional group and can be removed by conventional chemical or enzymatic steps to reestablish the carboxyl functional group. The particular protecting group employed is not critical.

The term “Prodrug” refers to an agent that is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. A prodrug may be converted into the parent drug by various mechanisms, including enzymatic processes and metabolic hydrolysis. See Harper, “Drug Latentiation” in Jucker, ed. Progress in Drug Research 4:221-294 (1962); Morozowich et al., “Application of Physical Organic Principles to Prodrug Design” in E. B. Roche ed. Design of Biopharmaceutical Properties through Prodrugs and Analogs, APHA Acad. Pharm. Sci. (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of .beta.-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Adv. Drug Delivery Rev. 8(1): 1-38 (1992); Fleisher et al. “Improved oral drug delivery: solubility limitations overcome by the use of prodrugs”, Adv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, J. Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” J. Chem. Soc., Chem. Commun., 875-877 (1991); Friis and Bundgaard, “Prodrugs of phosphates and phosphonates: Novel lipophilic alpha-acyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989).

The terms “halogen”, “halide” or “halo” include fluorine, chlorine, bromine, and iodine.

The terms “alkyl” and “substituted alkyl” are interchangeable and include substituted and unsubstituted C₁-C₁₀ straight chain saturated aliphatic hydrocarbon groups, substituted and unsubstituted C₂-C₁₀ straight chain unsaturated aliphatic hydrocarbon groups, substituted and unsubstituted C₄-C₁₀ branched saturated aliphatic hydrocarbon groups, substituted and unsubstituted C₄-C₁₀ branched unsaturated aliphatic hydrocarbon groups, substituted and unsubstituted C₃-C₈ cyclic saturated aliphatic hydrocarbon groups, substituted and unsubstituted C₅-C₈ cyclic unsaturated aliphatic hydrocarbon groups having the specified number of carbon atoms. For example, the definition of “alkyl” shall include but is not limited to: methyl (Me), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group comprising halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₂₁ and —O₀₋₁₀alkylCONR₂₂R₂₃ wherein R₂₁, R₂₂ and R₂₃ are independently selected from hydrogen, alkyl, aryl, or R₂₂ and R₂₃ are taken together with the nitrogen to which they are attached forming a saturated cyclic or unsaturated cyclic system containing 3 to 8 carbon atoms with at least one substituent as defined herein.

The term “alkyloxy” (e.g. methoxy, ethoxy, propyloxy, allyloxy, cyclohexyloxy) represents a substituted or unsubstituted alkyl group as defined above having the indicated number of carbon atoms attached through an oxygen bridge. The term “alkyloxyalkyl” represents an alkyloxy group attached through an alkyl or substituted alkyl group as defined above having the indicated number of carbon atoms.

The term “alkyloxycarbonyl” (e.g. methoxycarbonyl, ethoxycarbonyl, tert-butoxycarbonyl, allyloxycarbonyl) represents a substituted or unsubstituted alkyloxy group as defined above having the indicated number of carbon atoms attached through a carbonyl bridge.

The term “alkylthio” (e.g. methylthio, ethylthio, propylthio, cyclohexenylthio and the like) represents a substituted or unsubstituted alkyl group as defined above having the indicated number of carbon atoms attached through a sulfur bridge. The term “alkylthioalkyl” represents an alkylthio group attached through an alkyl or substituted alkyl group as defined above having the indicated number of carbon atoms.

The term “alkylamino” (e.g. methylamino, diethylamino, butylamino, N-propyl-N-hexylamino, (2-cyclopentyl)propylamino, hexenylamino, and the like) represents one or two substituted or unsubstituted alkyl groups as defined above having the indicated number of carbon atoms attached through an amine bridge. The substituted or unsubstituted alkyl groups maybe taken together with the nitrogen to which they are attached forming a saturated cyclic or unsaturated cyclic system containing 3 to 10 carbon atoms with at least one substituent as defined above. The term “alkylaminoalkyl” represents an alkylamino group attached through a substituted or unsubstituted alkyl group as defined above having the indicated number of carbon atoms.

The term “alkylhydrazino” (e.g. methylhydrazino, diethylhydrazino, butylhydrazino, (2-cyclopentyl)propylhydrazino, cyclohexanehydrazino, and the like) represents one or two substituted or unsubstituted alkyl groups as defined above having the indicated number of carbon atoms attached through a nitrogen atom of a hydrazine bridge. The substituted or unsubstituted alkyl groups maybe taken together with the nitrogen to which they are attached forming a saturated cyclic or unsaturated cyclic system containing 3 to 10 carbon atoms with at least one substituent as defined above. The term “alkylhydrazinoalkyl” represents an alkylhydrazino group attached through a substituted or unsubstituted alkyl group as defined above having the indicated number of carbon atoms.

The term “alkylcarbonyl” (e.g. cyclooctylcarbonyl, pentylcarbonyl, 3-hexenylcarbonyl and the like) represents a substituted or unsubstituted alkyl group as defined above having the indicated number of carbon atoms attached through a carbonyl group. The term “alkylcarbonylalkyl” represents an alkylcarbonyl group attached through a substituted or unsubstituted alkyl group as defined above having the indicated number of carbon atoms.

The term “alkylcarboxy” (e.g. heptylcarboxy, cyclopropylcarboxy, 3-pentenylcarboxy and the like) represents an alkylcarbonyl group as defined above wherein the carbonyl is in turn attached through an oxygen. The term “alkylcarboxyalkyl” represents an alkylcarboxy group attached through an alkyl group as defined above having the indicated number of carbon atoms.

The term “alkylcarbonylamino” (e.g. hexylcarbonylamino, cyclopentylcarbonyl-aminomethyl, methylcarbonylaminophenyl and the like) represents an alkylcarbonyl group as defined above wherein the carbonyl is in turn attached through the nitrogen atom of an amino group. The nitrogen group may itself be substituted with a substituted or unsubstituted alkyl or aryl group. The term “alkylcarbonylaminoalkyl” represents an alkylcarbonylamino group attached through a substituted or unsubstituted alkyl group as defined above having the indicated number of carbon atoms.

The term “alkylcarbonylhydrazino” (e.g. ethylcarbonylhydrazino, tert-butylcarbonylhydrazino and the like) represents an alkylcarbonyl group as defined above wherein the carbonyl is in turn attached through the nitrogen atom of a hydrazino group.

The term “aryl” represents an unsubstituted, mono-, di- or trisubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphtyl and the like). The aryl substituents are independently selected from the group comprising halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₂₁, and —C₀₋₁₀alkylCONR₂₂R₂₃ wherein R₂₁, R₂₂ and R₂₃ are independently selected from hydrogen, alkyl, aryl or R₂₂ and R₂₃ are taken together with the nitrogen to which they are attached forming a saturated cyclic or unsaturated cyclic system containing 3 to 8 carbon atoms with at least one substituent as defined above.

The definition of “aryl” includes but is not limited to phenyl, biphenyl, naphthyl, dihydronaphthyl, tetrahydronaphthyl, indenyl, indanyl, azulenyl, anthryl, phenanthryl, fluorenyl, pyrenyl and the like.

The term “arylalkyl” (e.g. (4-hydroxyphenyl)ethyl, (2-aminonaphthyl)hexenyl and the like) represents an aryl group as defined above attached through a substituted or unsubstituted alkyl group as defined above having the indicated number of carbon atoms.

The term “arylcarbonyl” (e.g. 2-thiophenylcarbonyl, 3-methoxyanthrylcarbonyl and the like) represents an aryl group as defined above attached through a carbonyl group.

The term “arylalkylcarbonyl” (e.g. (2,3-dimethoxyphenyl)propylcarbonyl, (2-chloronaphthyl)pentenyl-carbonyl and the like) represents an arylalkyl group as defined above wherein the alkyl group is in turn attached through a carbonyl.

The term “aryloxy” (e.g. phenoxy, naphthoxy, 3-methylphenoxy, and the like) represents an aryl or substituted aryl group as defined above having the indicated number of carbon atoms attached through an oxygen bridge. The term “aryloxyalkyl” represents an aryloxy group attached through a substituted or unsubstituted alkyl group as defined above having the indicated number of carbon atoms.

The term “aryloxycarbonyl” (e.g. phenoxycarbonyl, naphthoxycarbonyl) represents a substituted or unsubstituted aryloxy group as defined above having the indicated number of carbon atoms attached through a carbonyl bridge.

The term “arylthio” (e.g. phenylthio, naphthylthio, 3-bromophenylthio, and the like) represents an aryl or substituted aryl group as defined above having the indicated number of carbon atoms attached through a sulfur bridge. The term “arylthioalkyl” represents an arylthio group attached through a substituted or unsubstituted alkyl group as defined above having the indicated number of carbon atoms.

The term “arylamino” (e.g. phenylamino, diphenylamino, naphthylamino, N-phenyl-N-naphthylamino, o-methylphenylamino, p-methoxyphenylamino, and the like) represents one or two aryl groups as defined above having the indicated number of carbon atoms attached through an amine bridge. The term “arylaminoalkyl” represents an arylamino group attached through a substituted or unsubstituted alkyl group as defined above having the indicated number of carbon atoms. The term “arylalkylamino” represents an aryl group attached through an alkylamino group as defined above having the indicated number of carbon atoms. The term “N-aryl-N-alkylamino” (e.g. N-phenyl-N-methylamino, N-naphthyl-N-butylamino, and the like) represents one aryl and one a substituted or unsubstituted alkyl group as defined above having the indicated number of carbon atoms independently attached through an amine bridge.

The term “arylhydrazino” (e.g. phenylhydrazino, naphthylhydrazino, 4-methoxyphenylhydrazino, and the like) represents one or two aryl groups as defined above having the indicated number of carbon atoms attached through a hydrazine bridge. The term “arylhydrazinoalkyl” represents an arylhydrazino group attached through a substituted or unsubstituted alkyl group as defined above having the indicated number of carbon atoms. The term “arylalkylhydrazino” represents an aryl group attached through an alkylhydrazino group as defined above having the indicated number of carbon atoms. The term “N-aryl-N-alkylhydrazino” (e.g. N-phenyl-N-methylhydrazino, N-naphthyl-N-butylhydrazino, and the like) represents one aryl and one a substituted or unsubstituted alkyl group as defined above having the indicated number of carbon atoms independently attached through an amine atom of a hydrazine bridge.

The term “arylcarboxy” (e.g. phenylcarboxy, naphthylcarboxy, 3-fluorophenylcarboxy and the like) represents an arylcarbonyl group as defined above wherein the carbonyl is in turn attached through an oxygen bridge. The term “arylcarboxyalkyl” represents an arylcarboxy group attached through a substituted or unsubstituted alkyl group as defined above having the indicated number of carbon atoms.

The term “arylcarbonylamino” (e.g. phenylcarbonylamino, naphthylcarbonylamino, 2-methylphenylcarbonylamino and the like) represents an arylcarbonyl group as defined above wherein the carbonyl is in turn attached through the nitrogen atom of an amino group. The nitrogen group may itself be substituted with an a substituted or unsubstituted alkyl or aryl group. The term “arylcarbonylaminoalkyl” represents an arylcarbonylamino group attached through a substituted or unsubstituted alkyl group as defined above having the indicated number of carbon atoms. The nitrogen group may itself be substituted with a substituted or unsubstituted alkyl or aryl group.

The term “arylcarbonylhydrazino” (e.g. phenylcarbonylhydrazino, naphthylcarbonylhydrazino, and the like) represents an arylcarbonyl group as defined above wherein the carbonyl is in turn attached through the nitrogen atom of a hydrazino group.

The terms “heteroaryl”, “heterocycle” or “heterocyclic” refers to a monovalent unsaturated group having a single ring or multiple condensed rings, from 1 to 8 carbon atoms and from 1 to 4 hetero atoms selected from nitrogen, sulfur or oxygen within the ring. The heteroaryl groups in this invention can be optionally substituted with 1 to 3 substituents selected from the group comprising: halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₂₁, and —C₀₋₁₀alkylCONR₂₂R₂₃ wherein R₂₁, R₂₂ and R₂₃ are independently selected from hydrogen, alkyl, aryl, or R₂₂ and R₂₃ are taken together with the nitrogen to which they are attached forming a saturated cyclic or unsaturated cyclic system containing 3 to 8 carbon atoms with at least one substituent as defined above.

The definition of “heteroaryl” includes but is not limited to thienyl, benzothienyl, isobenzothienyl, 2,3-dihydrobenzothienyl, furyl, pyranyl, benzofuranyl, isobenzofuranyl, 2,3-dihydrobenzofuranyl, pyrrolyl, pyrrolyl-2,5-dione, 3-pyrrolinyl, indolyl, isoindolyl, 3H-indolyl, indolinyl, indolizinyl, indazolyl, phthalimidyl (or isoindoly-1,3-dione), imidazolyl, 2H-imidazolinyl, benzimidazolyl, pyridyl, pyrazinyl, pyradazinyl, pyrimidinyl, triazinyl, quinolyl, isoquinolyl, 4H-quinolizinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, chromanyl, benzodioxolyl, piperonyl, purinyl, pyrazolyl, triazolyl, tetrazolyl, thiazolyl, isothiazolyl, benzthiazolyl, oxazolyl, isoxazolyl, benzoxazolyl, oxadiazolyl, thiadiazolyl, pyrrolidinyl-2,5-dione, imidazolidinyl-2,4-dione, 2-thioxo-imidazolidinyl-4-one, imidazolidinyl-2,4-dithione, thiazolidinyl-2,4-dione, 4-thioxo-thiazolidinyl-2-one, piperazinyl-2,5-dione, tetrahydro-pyridazinyl-3,6-dione, 1,2-dihydro-[1,2,4,5]tetrazinyl-3,6-dione, [1,2,4,5]tetrazinanyl-3,6-dione, dihydro-pyrimidinyl-2,4-dione, pyrimidinyl-2,4,6-trione, 1H-pyrimidinyl-2,4-dione, 5-iodo-1H-pyrimidinyl-2,4-dione, 5-chloro-1H-pyrimidinyl-2,4-dione, 5-methyl-1H-pyrimidinyl-2,4-dione, 5-isopropyl-1H-pyrimidinyl-2,4-dione, 5-propynyl-1H-pyrimidinyl-2,4-dione, 5-trifluoromethyl-1H-pyrimidinyl-2,4-dione, 6-amino-9H-purinyl, 2-amino-9H-purinyl, 4-amino-1H-pyrimidinyl-2-one, 4-amino-5-fluoro-1H-pyrimidinyl-2-one, 4-amino-5-methyl-1H-pyrimidinyl-2-one, 2-amino-1,9-dihydro-purinyl-6-one, 1,9-dihydro-purinyl-6-one, 1H-[1,2,4]triazolyl-3-carboxylic acid amide, 2,6-diamino-N₆-cyclopropyl-9H-purinyl, 2-amino-6-(4-methoxyphenylsulfanyl)-9H-purinyl, 5,6-dichloro-1H-benzoimidazolyl, 2-isopropylamino-5,6-dichloro-1H-benzoimidazolyl, 2-bromo-5,6-dichloro-1H-benzoimidazolyl, and the like. For the purposes of this application, the terms “heteroaryl”, “heterocycle” or “heterocyclic” do not include carbohydrate rings (i.e. mono- or oligosaccharides).

The term “saturated heterocyclic” represents an unsubstituted, mono-, di- or trisubstituted monocyclic, polycyclic saturated heterocyclic group covalently attached at any ring position capable of forming a stable covalent bond, certain preferred points of attachment being apparent to those skilled in the art (e.g., 1-piperidinyl, 4-piperazinyl and the like).

The saturated heterocyclic substituents are independently selected from the group comprising halo, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N-—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₂₁, and —C₀₋₁₀alkylCONR₂₂R₂₃ wherein R₂₁, R₂₂ and R₂₃ are independently selected from hydrogen, alkyl, aryl, or R₂₂ and R₂₃ are taken together with the nitrogen to which they are attached forming a saturated cyclic or unsaturated cyclic system containing 3 to 8 carbon atoms with at least one substituent as defined above.

The definition of saturated heterocyclic includes but is not limited to pyrrolidinyl, pyrazolidinyl, piperidinyl, 1,4-dioxanyl, morpholinyl, 1,4-dithienyl, thiomorpholinyl, piperazinyl, quinuclidinyl, and the like.

The term “alpha-beta-unsaturated carbonyl” refers to a molecule that has a carbonyl group directly attached to a double or triple bonded carbon and which would be obvious to one of ordinary skill and knowledge in the art. The definition of alpha-beta-unsaturated carbonyl includes but is not limited to acrolein, methyl vinyl ketone, and the like.

The term “acetal” refers to a molecule that contains a carbon atom C₁ that is directly attached to a hydrogen atom (H₁), a substituted carbon atom (C₂) and two oxygen atoms (O₁ and O₂). These oxygen atoms are in turn attached to other substituted carbon atoms (C₃ and C₄), which would be obvious to one of ordinary skill and knowledge in the art. The definition of acetal includes but is not limited to 1,1-dimethoxypropane, 1,1-bis-allyloxybutane and the like.

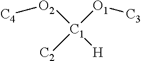

The term “cyclic acetal” refers to an acetal as defined above where C₃ and C₄, together with the oxygen atoms to which they are attached, combine thru an alkyl bridge to form a 5- to 10-membered ring, which would be obvious to one of ordinary skill and knowledge in the art. The definition of cyclic acetal includes but is not limited to 2-methyl-[1,3]dioxolane, 2-ethyl-[1,3]dioxane, 2-phenyl-[1,3]dioxane, 2 2-phenyl-hexahydro-pyrano[3,2-d][1,3]dioxine and the like.

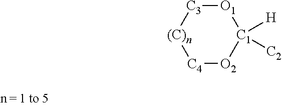

The term “ketal” refers to a molecule that contains a carbon atom C₁ that is directly attached to two substituted carbon atom (C₂ and C₃) and two oxygen atoms (O₁ and O₂). These oxygen atoms are in turn attached to other substituted carbon atoms (C₄ and C₅), which would be obvious to one of ordinary skill and knowledge in the art. The definition of acetal includes but is not limited to 2,2-dimethoxy-butane, 3,3-diethoxy-pentane and the like.

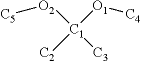

The term “cyclic ketal” refers to a ketal as defined above where C₄ and C₅, together with the oxygen atoms to which they are attached, combine thru an alkyl bridge to form a 5- to 10-membered ring, which would be obvious to one of ordinary skill and knowledge in the art. The definition of cyclic acetal includes but is not limited to 2,2,4,5-tetramethyl-[1,3]dioxolane, 2,2-diethyl-[1,3]dioxepane, 2,2-dimethyl-hexahydro-pyrano[3,2-d][1,3]dioxine and the like.

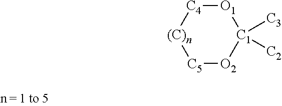

In one embodiment of the invention, there are provided compounds having the structural formula A:

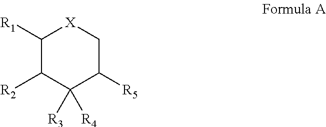

wherein:
a) X is selected from the group consisting of oxygen, sulfur, and —NR₆
b) R₁ is selected from the group consisting of C₁₋₁₀ substituted alkyl, and —CH₂OR₁₁
c) R₂ is selected from the group consisting of hydrogen, halogen, —OR₁₂, —SR₁₂, and —NHR₁₂
d) R₃ and R₄ are independently selected from the group consisting of hydrogen, halogen, azido, —CN, C₁₋₁₀ alkylcarboxy, C₁₋₁₀ arylcarboxy, and —OSO₂R₇ with the proviso that R₃ and R₄ cannot both be hydrogen,
e) R₅ is heteroaryl, saturated heterocyclic, or —NR₈R₉,
f) R₆ is selected from the group consisting of hydrogen, amino protecting group, C₁₋₁₀ alkyl, C₁₋₁₀ substituted alkyl, aryl, C₁₋₁₀ alkylcarbonyl, arylcarbonyl, C₁₋₁₀ alkyloxycarbonyl, aryloxycarbonyl, and —SO₂R₁₀
g) R₈ and R₉ are independently selected from the group consisting of hydrogen, and C₁₋₁₀ alkyl, or R₈ and R₉, together with the nitrogen atom to which they are attached, combine to form a saturated heterocyclic or heteroaryl ring;
h) R₇ and R₁₀ are independently selected from the group consisting of C₁₋₁₀ alkyl, C₁₋₁₀ substituted alkyl, aryl and substituted aryl,
i) R₁₁ is selected from the group consisting of hydrogen, hydroxyl protecting group, —P(O)(OR₁₅)(OR₁₆), and —CH₂P(O)(OR₁₅)(OR₁₆),
j) R₁₂ is selected from the group consisting of hydrogen, —PR₁₃R₁₄, hydroxyl protecting group if R₂ is —OR₁₂, thiol protecting group if R₂ is —SR₁₂, and amino protecting group if R₂ is —NHR₁₂, or if R₁ is —CH₂OR₁₁ and R₂ is —OR₁₂, then R₁₁ and R₁₂ together with the oxygen atoms to which they are attached, combine to form a cyclic acetal or ketal,
k) R₁₃ and R₁₄ are independently selected from the group consisting of —NR₈R₉, and —OCH₂CH₂CN,
l) R₁₅ and R₁₆ are independently selected from the group consisting of hydrogen, C₁₋₁₀ alkyl

or a pharmaceutically acceptable salt of any of the foregoing.

Invention compounds having structure A include

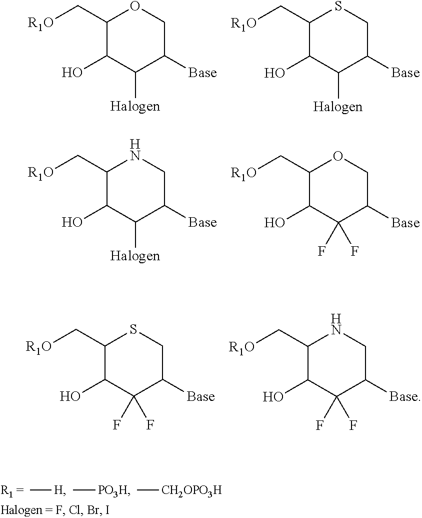

### Base:

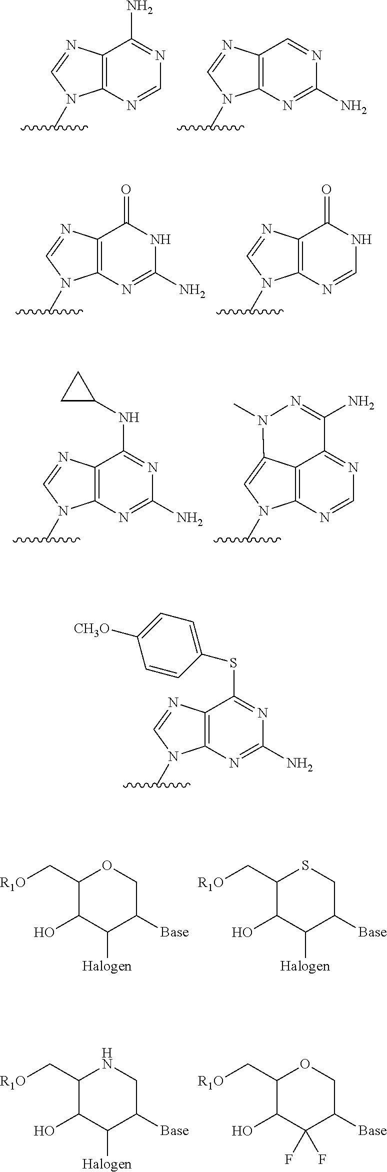

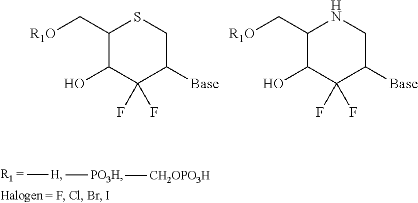

### Base:

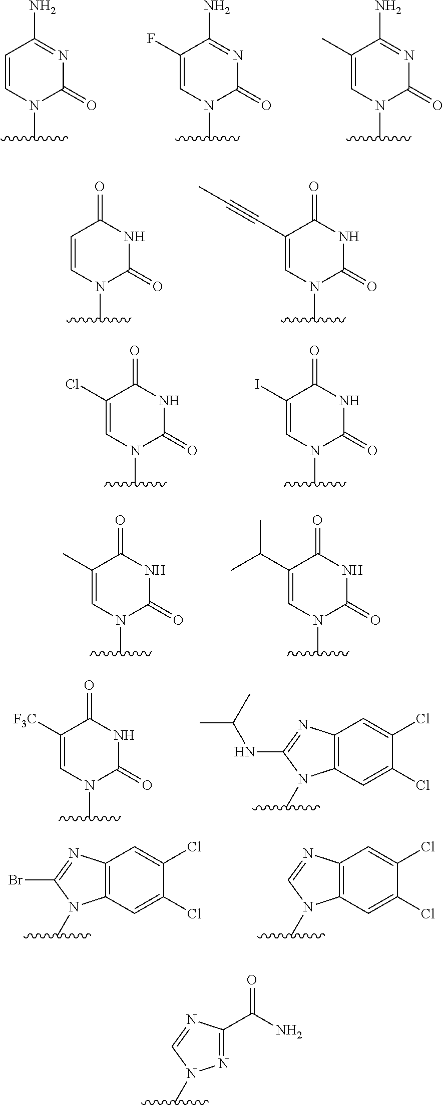

and pharmaceutically acceptable salts of the forgoing.

In another embodiment of the invention, there are provided pharmaceutical compositions comprising at least one of the compounds of the invention, as well as pharmaceutically acceptable pro-drugs and salts of such compounds, in a pharmaceutically acceptable vehicle, for enteral, parenteral, topical or ocular administration.

In another embodiment of the invention, there are provided pharmaceutical compositions comprising at least one of the compounds of the invention, in a pharmaceutically acceptable vehicle, for the treatment or prophylaxis of viral infections and the like.

In another embodiment of the invention, there are provided pharmaceutical compositions comprising at least one of the compounds of the invention, in a pharmaceutically acceptable vehicle, for the treatment or prophylaxis of bacterial infections and the like.

In another embodiment of the invention, there are provided pharmaceutical compositions comprising at least one of the compounds of the invention, in a pharmaceutically acceptable vehicle, for the treatment or prophylaxis of fungal infections and the like.

In another embodiment of the invention, there are provided pharmaceutical compositions comprising at least one of the compounds of the invention, in a pharmaceutically acceptable vehicle, for use in antisense therapy, such as for example, in the treatment or prophylaxis of cancer, diabetes and other diseases of genetic origin.

In another embodiment of the invention, there are provided pharmaceutical compositions comprising at least one of the compounds of the invention, in a pharmaceutically acceptable vehicle, for the treatment of carcinomas. Examples of carcinomas include mammary cancer, prostate cancer, kidney cancer, Karposi's sarcoma, colon cancer, cervical cancer, lung cancer, cutaneous T-cell lymphoma, cancer of the head and neck, cancers of the aerodigestive pathway, skin cancer, bladder cancer, sarcomas, leukoplakias, acute promyelocytic leukemia, and the like.

In another embodiment of the invention, there are provided pharmaceutical compositions comprising at least one of the compounds of the invention in combination with other therapeutic agents and to methods of treating diseases and/or conditions using the same. Example of diseases and/or conditions include cancer, mammary cancer, prostate cancer, kidney cancer, Karposi's sarcoma, colon cancer, cervical cancer, lung cancer, cutaneous T-cell lymphoma, cancer of the head and neck, cancers of the aerodigestive pathway, skin cancer, bladder cancer, sarcomas, leukoplakias, acute promyelocytic leukemia and the like. Examples of other therapeutic agents include Busulfan, Carboplatin, Cisplatin, Cyclophosphamide, Cytosine arabinoside, Etoposide, 5-Fluorouracil, Melphalan, Methotrexate, Mitoxantrone, Taxol, Interferon, Fareston, Arzoxifene, Evista, Tamoxifen, and the like.

In another embodiment of the invention, there are provided methods of modulating the expression of enzymes, proteins, nuclear factors or receptors in cells or tissues comprising contacting said cells or tissues with one or more of the compounds or compositions of the invention.

In another embodiment of the invention, there are provided methods of treating a mammalian subject, particularly a human, suspected of having or being prone to a disease or condition associated with expression of said enzymes, proteins, nuclear factors or receptors by administering a therapeutically or prophylactically effective amount of one or more of the compounds or compositions of the invention.

In another embodiment of the invention, there are provided nucleic acid probes constructed from compounds of formula A.

In another embodiment of the invention, there are provided methods of using nucleic acid probes constructed from compounds of formula A for the identification and quantification of a bacterium, virus or any other organism in sputum, urine, blood, tissue sections, food, soil, water and the like.

Pharmaceutical compositions containing the nucleoside analogs of the invention as an active ingredient can take the form of tablets, capsules, powders, suspensions, solutions, emulsions as well as salves and creams, and can be used for parenteral (intravenous, intradermal, intramuscular, intrathecal etc.) injections, oral, rectal, intravaginal and intranasal administering or for local application (for instance oligonucleotide skin injuries, mucosa and eyes). Such compositions can be prepared by combining the active ingredient(s) with pharmaceutically acceptable excipients normally used for this purpose. Such excipients can comprise aqueous and non-aqueous solvents, stabilizers, suspension agents, dispersing agents, moisturizers and the like, and will be known to the skilled person in the pharmaceutical field. The composition may further contain likewise suitable additives such as for instance polyethylene glycols and, if necessary, colorants, fragrances and the like.

The pharmaceutical compositions will preferably contain at least 0.1 volume % by weight of the active ingredient. The actual concentration will depend oligonucleotide the disease and the chosen administering route. In general this concentration will lie between 0.1 and 100% for the above applications and indications. The dose of the active ingredient to be administered can further vary between 0.1 mg and 100 mg per kg body weight, preferably between 0.1 mg and 50 mg per kg body weight, and most preferably between 0.5 mg and 20 mg per kg body weight.

The desired dose is preferably presented in the form of two, three, four, five, six or more sub-doses which are administered at appropriate intervals per day. These sub-doses can be administered in the form of dosage units containing for instance from 1 to 1500 mg, preferably from 5 to 1000 mg and most preferably from 10 to 700 mg active constituent per dosage unit, and if the condition of the patient requires the dose can, by way of alternative, be administered as a continuous infusion.

### General Methods of Preparation

As used herein, the following abbreviations have the following meanings: Me refers to methyl (CH₃—), Et refers to ethyl (CH₃CH₂—), i-Pr refers to isopropyl ((CH₃)₂CH₂—), t-Bu or tert-butyl refers to tertiary butyl ((CH₃)₃CH—), Ph refers to phenyl, Bn refers to benzyl (PhCH₂—), Bz refers to benzoyl (PhCO—), MOM refers to methoxymethyl, Ac refers to acetyl, TMS refers to trimethylsilyl, TBS refers to ter-butyldimethylsilyl, Ms refers to methanesulfonyl (CH₃SO₂—), Ts refers to p-toluenesulfonyl (p-CH₃PhSO₂—), Tf refers to trifluoromethanesulfonyl (CF₃SO₂—), TfO refers to trifluoromethanesulfonate (CF₃SO₃—), DMF refers to N,N-dimethylformamide, DCM refers to dichloromethane (CH₂Cl₂), THF refers to tetrahydrofuran, EtOAc refers to ethyl acetate, Et₂O refers to diethyl ether, MeCN refers to acetonitrile (CH₃CN), NMP refers to 1-N-methyl-2-pyrrolidinone, DMA refers to N,N-dimethylacetamide, DMSO refers to dimethylsulfoxide, DCC refers to 1,3-dicyclohexyldicarbodiimide, EDCI refers to 1-(3-dimethylaminopropyl)-3-ethylcarbodiimide, Boc refers to tert-butylcarbonyl, Fmoc refers to 9-fluorenylmethoxycarbonyl, TBAF refers to tetrabutylammonium fluoride, TBAI refers to tetrabutylammonium iodide, TMEDA refers to N,N,N,N-tetramethylethylene diamine, Dess-Martin periodinane or Dess Martin reagent refers to 1,1,1-triacetoxy-1,1-dihydro-1,2-benziodoxol-3(1H)-one, DMAP refers to 4-N,N-dimethylaminopyridine, (i-Pr)₂NEt or DIEA or Hunig's base refers to N,N-diethylisopropylamine, DBU refers to 1,8-Diazabicyclo[5.4.0]undec-7-ene, (DHQ)₂AQN refers to dihydroquinine anthraquinone-1,4-diyl diether, (DHQ)₂PHAL refers to dihydroquinine phthalazine-1,4-diyl diether, (DHQ)₂PYR refers to dihydroquinine 2,5-diphenyl-4,6-pyrimidinediyl diether, (DHQD)₂AQN refers to dihydroquinidine anthraquinone-1,4-diyl diether, (DHQD)₂PHAL refers to dihydroquinidine phthalazine-1,4-diyl diether, (DHQD)₂PYR refers to dihydroquinidine 2,5-diphenyl-4,6-pyrimidinediyl diether, LDA refers to lithium diisopropylamide, LiTMP refers to lithium 2,2,6,6-tetramethylpiperidinamide, n-BuLi refers to n-butyllithium, t-BuLi refers to tert-butyl lithium, IBA refers to 1-hydroxy-1,2-benziodoxol-3(1H)-one 1-oxide, OsO₄ refers to osmium tetroxide, m-CPBA refers to meta-chloroperbenzoic acid, DMD refers to dimethyl dioxirane, PDC refers to pyridinium dichromate, NMO refers to N-methyl morpholine-N-oxide, NaHMDS refers to sodium hexamethyldisilazide, LiHMDS refers to lithium hexamethyldisilazide, HMPA refers to hexamethylphosphoramide, TMSCl refers to trimethylsilyl chloride, TMSCN refers to trimethylsilyl cyanide, TBSCl refers to tert-butyldimethylsilyl chloride, TFA refers to trifluoroacetic acid, TFAA refers to trifluoroacetic anhydride, AcOH refers to acetic acid, Ac₂O refers to acetic anhydride, AcCl refers to acetyl chloride, TsOH refers to p-toluenesulfonic acid, TsCl refers to p-toluenesulfonyl chloride, MBHA refers to 4-methylbenzhydrylamine, BHA refers to benzhydrylamine, ZnCl₂ refers to zinc (II) dichloride, BF₃ refers to boron trifluoride, Y(OTf)₂ refers to yttrium (III) trifluoromethanesulfonate, Cu(BF₄)₂ refers to copper (II) tetrafluoroborate, LAH refers to lithium aluminum hydride (LiAlH₄), NaHCO₃ refers to sodium bicarbonate, K₂CO₃ refers to potassium carbonate, NaOH refers to sodium hydroxide, KOH refers to potassium hydroxide, LiOH refers to lithium hydroxide, HCl refers to hydrochloric acid, H₂SO₄ refers to sulfuric acid, MgSO₄ refers to magnesium sulfate, and Na₂SO₄ refers to sodium sulfate. ¹H NMR refers to proton nuclear magnetic resonance, ¹³C NMR refers to carbon 13 nuclear magnetic resonance, NOE refers to nuclear overhauser effect, NOESY refers to nuclear overhauser and exchange spectroscopy, COSY refers to homonuclear correlation spectroscopy, HMQC refers to proton detected heteronuclear multiplet-quantum coherence, HMBC refers to heteronuclear multiple-bond connectivity, s refers to singlet, br s refers to broad singlet, d refers to doublet, br d refers to broad doublet, t refers to triplet, q refers to quartet, dd refers to double doublet, m refers to multiplet, ppm refers to parts per million, IR refers to infrared spectrometry, MS refers to mass spectrometry, HRMS refers to high resolution mass spectrometry, EI refers to electron impact, FAB refers to fast atom bombardment, CI refers to chemical ionization, HPLC refers to high pressure liquid chromatography, TLC refer to thin layer chromatography, R_{f} refers to, Rₜ refers to retention time, GC refers to gas chromatography, min is minutes, h is hours, rt or RT is room temperature, g is grams, mg is milligrams, L is liters, mL is milliliters, mol is moles and mmol is millimoles.

For all of the following examples, standard work-up and purification methods can be utilized and will be obvious to those skilled in the art. Synthetic methodologies that make up the invention are shown in Schemes 1-10. These Schemes are intended to describe the applicable chemistry through the use of specific examples and are not indicative of the scope of the invention.

### EXAMPLES

The following examples are provided to further illustrate the advantages and features of the present invention, but are not intended to limit the scope of the invention.

### Example 1

### Preparation of (3S,4R,5R,6R) acetic acid 3,4,5-triacetoxy-tetrahydropyran-2-ylmethyl ester

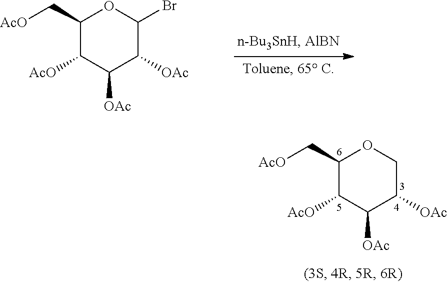

To a solution of acetobromo-α-D-glucose (15 g, 36.5 mmol) in toluene (200 mL) was added was added n-tributyltin hydride (9.8 mL, 36.4 mmol) and AIBN (100 mg, 0.61 mmol). The mixture was stirred at 65° C. for 1 hour. THF (30 mL), KF (20 mL of a 8.6M aqueous solution) and water (100 mL) were added, and the product was extracted with Et₂O three times. The combined organic layers were washed with brine, dried over MgSO₄, filtered, and concentrated in vacuo.

Yield: 9.88 g (82%); R_{f}=0.6 in 1:1 EtOAc-hexane. ¹H NMR (CDCl₃, 300 MHz): δ 2.04 (3H, s), 2.05 (6H, s), 2.11 (3H, s), 3.32 (1H, t), 3.61 (1H, ddd), 4.17 (3H, m), 5.02 (1H, m), 5.04 (1H, m), 5.21 (1H, t).

### Example 2

### Preparation of (4aR,7S,8R,8aS) 2-phenyl-hexahydropyrano[3,2-d][1,3]dioxine-7,8-diol

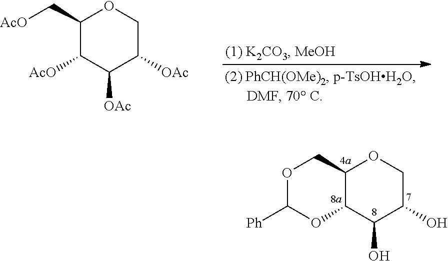

To a solution of (3S,4R,5R,6R) acetic acid 3,4,5-triacetoxy-tetrahydropyran-2-ylmethyl ester (9.88 g, 29.8 mmol) in 50 mL of MeOH was added K₂CO₃ (206 mg, 1.5 mmol). The mixture was stirred at room temperature for 48 hours. Toluene was added and the solvent was removed in vacuo. The residue was dissolved in 50 mL of DMF. PhCH(OMe)₂ (5.6 mL, 37.3 mmol) and p-TsOH monohydrate (1.7 g, 8.9 mmol) were added. The mixture was placed on a rotary evaporator under a mild vacuum and heated to 70° C. for 1 hour. NEt₃ (3 mL) was added and the solvent was removed in vacuo. The crude product was purified by flash chromatography and dried in vacuo overnight to give (4aR,7S,8R,8aS) 2-phenyl-hexahydropyrano[3,2-d][1,3]dioxine-7,8-diol.

Yield: 6.81 g (91% over 2 steps); R_{f}=0.65 in EtOAc. ¹H NMR (CDCl₃, 300 MHz): δ 2.87 (1H, br s), 3.22 (1H, br s), 3.30 (1H, t), 3.36 (1H, m), 3.44 (1H, m), 3.65-3.75 (3H, m), 3.98 (1H, dd), 4.31 (1H, dd), 5.51 (1H, s), 7.37-7.51 (5H, m).

### Example 3

### Preparation of (4aR,7S,8R,8aS) acetic acid 8-hydroxy-2-phenyl-hexahydropyrano[3,2-d][1,3]dioxin-7-yl ester and (4aR,7S,8R,8aR) acetic acid 7-hydroxy-2-phenyl-hexahydropyrano[3,2-d][1,3]dioxin-8-yl ester

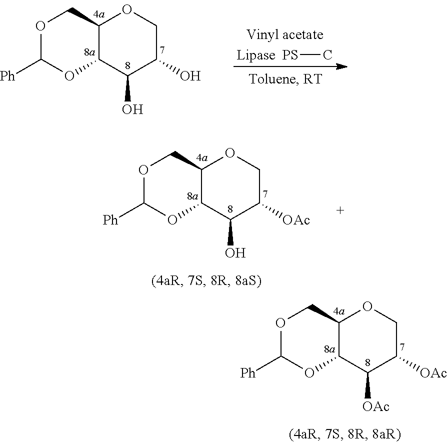

To a solution of (4aR,7S,8R,8aS) 2-phenyl-hexahydropyrano[3,2-d][1,3]dioxine-7,8-diol (6.81 g, 27 mmol) in toluene (200 ml) was added vinyl acetate (5 mL, 54 mmol) and Lipase PS—C “Amano” I (2.5 g). The mixture was stirred at room temperature overnight, filtered and concentrated in vacuo. The isomers (4aR,7S,8R,8aS) acetic acid 8-hydroxy-2-phenyl-hexahydropyrano[3,2-d][1,3]dioxin-7-yl ester and (4aR,7S,8R,8aR) acetic acid 7-hydroxy-2-phenyl-hexahydropyrano[3,2-d][1,3]dioxin-8-yl ester were separated by flash chromatography. (4aR,7S,8R,8aS) acetic acid 8-hydroxy-2-phenyl-hexahydropyrano[3,2-d][1,3]dioxin-7-yl ester.

Yield: 5.10 g (64%); R_{f}=0.5 in 1:1 EtOAc-hexane. ¹H NMR (CDCl₃, 300 MHz): δ 2.13 (3H, s), 2.66 (1H, br s), 3.34 (1H, t), 3.40 (1H, m), 3.55 (1H, t), 3.72 (1H, t), 3.92 (1H, t), 4.14 (1H, dd), 4.35 (1H, dd), 4.95 (1H, m), 5.55 (1H, s), 7.36-7.52 (5H, m). (4aR,7S,8R,8aR) Acetic acid 7-hydroxy-2-phenyl-hexahydropyrano[3,2-d][1,3]dioxin-8-yl ester. Yield: 1.96 g (25%).

### Example 4

### Preparation of (4aR,7S,8S,8aS) acetic acid 8-fluoro-2-phenyl-hexahydropyrano[3,2-d][1,3]dioxin-7-yl ester and (4aR,7S,8S,8aR) 8-fluoro-2-phenyl-hexahydropyrano[3,2-d][1,3]dioxin-7-ol

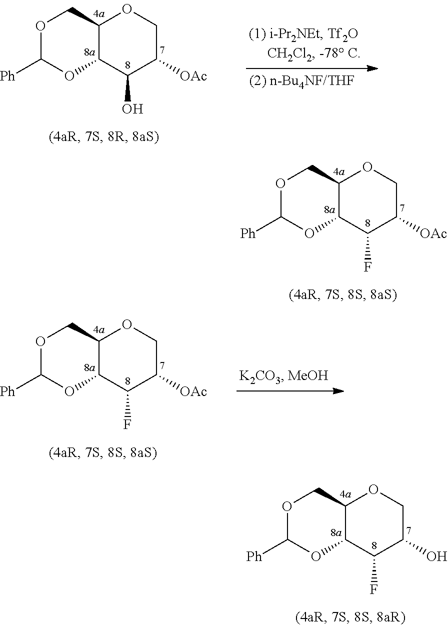

To a 20 mL CH₂Cl₂ solution of (4aR,7S,8R,8aS) acetic acid 8-hydroxy-2-phenyl-hexahydropyrano[3,2-d][1,3]dioxin-7-yl ester (1 g, 3.40 mmol) at −78° C. was added i-Pr₂NEt (1.8 mL, 10.3 mmol) and trifluoromethanesulfonic anhydride (0.8 mL, 4.8 mmol). The mixture was stirred at −78° C. for 15 minutes. Tetrabutylammonium fluoride (20 mL of a 1 M solution in THF, 20 mmol) was added, and the mixture was stirred at room temperature overnight. Water (50 mL) was added, and the mixture was extracted with Et₂O. The combined organic layers were washed with brine, dried over Na₂SO₄, filtered, and concentrated in vacuo to yield (4aR,7S,8S,8aS) acetic acid 8-fluoro-2-phenyl-hexahydropyrano[3,2-d][1,3]dioxin-7-yl ester. The crude isolate was dissolved in 20 mL of MeOH and K₂CO₃ (100 mg, 0.72 mmol) was added. The mixture was stirred at room temperature for 2 hours, and the solvent was removed in vacuo. Saturated aqueous NR₄Cl (20 mL) was added and the mixture was extracted with CH₂Cl₂. The combined organic layers were dried over Na₂SO₄, filtered, and concentrated in vacuo. The crude residue was purified by flash chromatography to give (4aR,7S,8S,8aR) 8-fluoro-2-phenyl-hexahydropyrano[3,2-d][1,3]dioxin-7-ol.

Yield: 430 mg (50% over 3 steps); R_{f}=0.4 in 1:1 EtOAc-hexane. ¹H NMR (CDCl₃, 300 MHz): δ 2.09 (1H, br d), 3.50-3.71 (3H, m), 3.76-4.00 (3H, m), 4.40 (1H, dd), 5.02-5.20 (1H, d), 5.55 (1H, s), 7.36-7.52 (5H, m).

### Example 5

### Preparation of (4aR,7S,8R,8aR) trifluoromethanesulfonic acid 8-fluoro-2-phenyl-hexahydropyrano[3,2-d][1,3]dioxin-7-yl ester

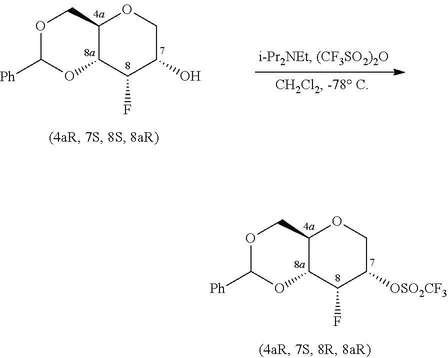

To a solution of (4aR,7S,8S,8aR) 8-fluoro-2-phenyl-hexahydropyrano[3,2-d][1,3]dioxin-7-ol (40 mg, 0.157 mmol) in 5 mL of CH₂Cl₂ was added i-Pr₂NEt (0.19 mL, 1.09 mmol) and trifluoromethanesulfonic anhydride (0.08 mL, 0.476 mmol) at −78° C. The mixture was stirred at −78° C. for 15 minutes, concentrated in vacuo and purified by flash chromatography to give (4aR,7S,8R,8aR) trifluoromethanesulfonic acid 8-fluoro-2-phenyl-hexahydropyrano[3,2-d][1,3]dioxin-7-yl ester.

Yield: 57.7 mg (95%); R_{f}=0.65 (3:1 hexane/EtOAc). ¹H NMR (CDCl₃, 300 MHz) δ 3.57-3.73 (2H, m), 3.87 (1H, m), 3.93 (1H, t), 4.10 (1H, dd), 4.42 (1H, dd), 4.94 (1H, dddd), 5.19-5.38 (1H, d), 5.55 (1H, s), 7.37-7.50 (5H, m).

### Example 6

### Preparation of (4aR,7R,8S,8aR) 9-(8-fluoro-2-phenyl-hexahydropyrano[3,2-d][1,3]dioxin-7-yl)-9H-purin-6-ylamine

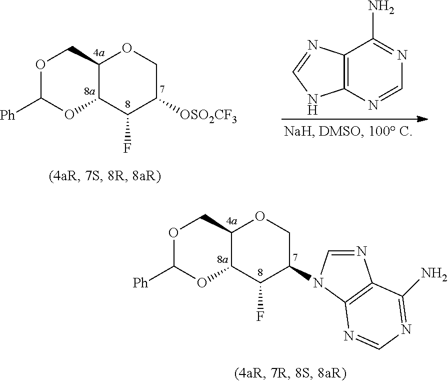

To a solution of (4aR,7S,8R,8aR) trifluoromethanesulfonic acid 8-fluoro-2-phenyl-hexahydropyrano[3,2-d][1,3]dioxin-7-yl ester (57.7 mg, 0.149 mmol) in 20 mL of DMSO was added adenine (1 g, 7.4 mmol) and NaH (178 mg, 7.4 mmol). The mixture was stirred at 100° C. overnight. Water was added to the mixture and the product was extracted with EtOAc three times. The combined organic fractions were washed with water and then brine, dried over Na₂SO₄, filtered and concentrated in vacuo. The crude product was purified by flash chromatography to give (4aR,7R,8S,8aR) 9-(8-fluoro-2-phenyl-hexahydropyrano[3,2-d][1,3]dioxin-7-yl)-9H-purin-6-ylamine.

Yield: 43.6 mg (79%); R_{f}=0.2 (EtOAc). ¹H NMR (CDCl₃, 300 MHz) δ 3.68-3.38 (2H, m), 4.15 (1H, m), 4.38-4.54 (3H, m), 4.99 (1H, m), 5.22-5.38 (1H, d), 5.50 (1H, s), 5.90 (2H, br s), 7.33-7.45 (5H, m), 8.36 (1H, s), 8.39 (1H, s).

### Example 7

### Preparation of (2R,3R,4S,5R) 5-(6-amino-purin-9-yl)-4-fluoro-2-hydroxymethyl-tetrahydropyran-3-ol

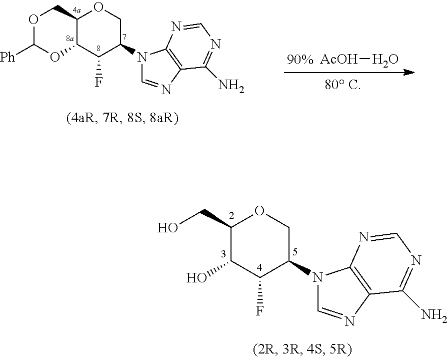

(4aR,7R,8S,8aR) 9-(8-fluoro-2-phenyl-hexahydropyrano[3,2-d][1,3]dioxin-7-yl)-9H-purin-6-ylamine (7.7 mg, 0.021 mmol) was dissolved in 3 mL of 9:1 acetic acid-water. The mixture was stirred at 80° C. for 2 hours, and was concentrated in vacuo. The residue was dissolved in MeOH and 3 drops of NEt₃ were added. The solvent was removed in vacuo, and the crude product was purified by PTLC to give (2R,3R,4S,5R) 5-(6-amino-purin-9-yl)-4-fluoro-2-hydroxymethyl-tetrahydropyran-3-ol.

Yield: 4.7 mg (80%); R_{f}=0.2 (10% MeOH—CH₂Cl₂). ¹H NMR (acetone-d₆, 300 MHz) δ 3.74-3.98 (4H, m), 4.25 (1H, dt), 4.37 (1H, dd), 4.46 (1H, br), 4.95 (1H, m), 5.08-5.24 (1H, ddd), 6.62 (2H, s), 8.21 (1H, s), 8.35 (1H, s).

### Example 8

### Preparation of (4aR,7R,8S,8aR) 4-amino-1-(8-fluoro-2-phenyl-hexahydropyrano[3,2-d][1,3]dioxin-7-yl)-1H-pyrimidin-2-one

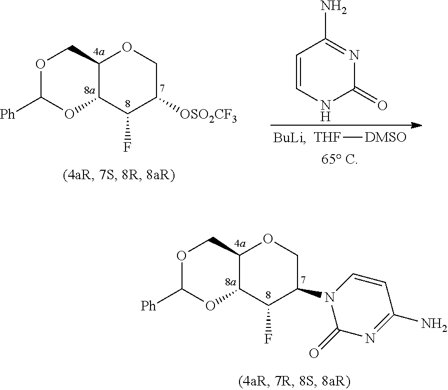

To a suspension of cytosine (300 mg, 2.7 mmol) in 5 mL of THF was added n-BuLi (0.97 mL of a 2.5M solution in hexane, 2.43 mmol) at room temperature. The mixture was cooled to 0° C. and DMSO (5 mL) was added. The reaction was then stirred at room temperature for 5 minutes to yield a clear solution. A solution of (4aR,7S,8R,8aR) trifluoromethanesulfonic acid 8-fluoro-2-phenyl-hexahydropyrano[3,2-d][1,3]dioxin-7-yl ester (11.8 mg, 0.031 mmol) in 2 mL of DMSO was added, and the mixture was stirred at 65° C. for 24 hours. After aqueous work-up ad extraction with EtOAc, the combined organic fractions were dried over Na₂SO₄, filtered and concentrated in vacuo. The crude product was purified by PTLC to give (4aR,7R,8S,8aR) 4-amino-1-(8-fluoro-2-phenyl-hexahydropyrano[3,2-d][1,3]dioxin-7-yl)-1H-pyrimidin-2-one.

Yield: 6.1 mg (58%); R_{f}=0.6 (EtOAc). ¹H NMR (CDCl₃, 300 MHz) δ 3.80 (1H, dt), 3.92-4.25 (4H, m), 4.36 (1H, dd), 5.00 (2H, br), 5.03-5.20 (1H, d), 5.33 (1H, t), 5.62 (1H, s), 6.15 (1H, d), 7.35-7.51 (5H, m), 8.02 (1H, d).

### Example 9

### Preparation of (2R,3R,4S,5R) 4-amino-1-(4-fluoro-5-hydroxy-6-hydroxymethyl-tetrahydropyran-3-yl)-1H-pyrimidin-2-one

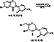

(4aR,7R,8S,8aR) 4-amino-1-(8-fluoro-2-phenyl-hexahydropyrano[3,2-d][1,3]dioxin-7-yl)-1H-pyrimidin-2-one (6.1 mg, 0.018 mmol) was dissolved in 3 mL of 9:1 acetic acid-water. and stirred at 80° C. for 1 hour. After concentration in vacuo, the crude product was purified by PTLC to give (2R,3R,4S,5R) 4-amino-1-(4-fluoro-5-hydroxy-6-hydroxymethyl-tetrahydropyran-3-yl)-1H-pyrimidin-2-one.

Yield: 3.2 mg (70%); R_{f}=0.15 (10% MeOH—CH₂Cl₂). 1H NMR (acetone-d₆, 300 MHz) δ 3.56 (1H, m), 3.70 (1H, m), 3.77-4.00 (4H, m), 4.85-5.02 (1H, dt), 5.17 (1H, m), 6.23 (1H, d), 6.32 (2H, br), 7.91 (1H, d).

### Example 10

### Reparation of (4aR,7R,8S,8aR) 6-chloro-9-(8-fluoro-2-phenyl-hexahydropyrano[3,2-d][1,3]dioxin-7-yl)-9H-purin-2-ylamine

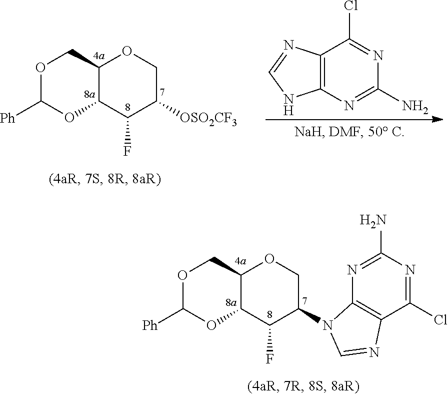

To (4aR,7S,8R,8aR) trifluoromethanesulfonic acid 8-fluoro-2-phenyl-hexahydropyrano[3,2-d][1,3]dioxin-7-yl ester (27.2 mg, 0.070 mmol) in 8 mL DMF was added 2-amino-6-chloropurine (300 mg, 1.8 mmol) and NaH (43 mg, 1.8 mmol). The mixture was stirred at 50° C. for 12 hours. After aqueous workup and extraction with Et₂O, the combined organic fractions were dried over Na₂SO₄, filtered and concentrated in vacuo. The crude product was purified by flash chromatography to give (4aR,7R,8S,8aR) 6-chloro-9-(8-fluoro-2-phenyl-hexahydropyrano[3,2-d][1,3]dioxin-7-yl)-9H-purin-2-ylamine.

Yield: 15.5 mg (54%); R_{f}=0.4 (1:1 EtOAc-hexane). ¹H NMR (CDCl₃, 300 MHz) δ 3.66 (1H, ddd), 3.78 (1H, dt), 4.06-4.18 (2H, m), 4.35-4.49 (2H, m), 4.81 (1H, m), 5.18 (2H, br), 5.18-5.35 (1H, d), 5.50 (1H, s), 7.33-7.45 (5H, m), 8.26 (1H, s).

### Example 11

### Preparation of (4aR,7R,8S,8aR) 2-amino-9-(8-fluoro-2-phenyl-hexahydropyrano[3,2-d][1,3]dioxin-7-yl)-1,9-dihydro-purin-6-one

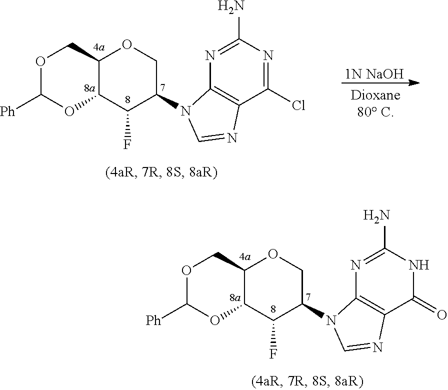

To a 3 mL dioxane solution of (4aR,7R,8S,8aR) 6-chloro-9-(8-fluoro-2-phenyl-hexahydropyrano[3,2-d][1,3]dioxin-7-yl)-9H-purin-2-ylamine (15.5 mg, 0.038 mmol) was added 3 mL of 1N NaOH. The mixture was stirred at 80° C. for 1 hour, acidified and extracted with EtOAc three times. The combined organic fractions were dried over Na₂SO₄, filtered and concentrated in vacuo. The crude product was purified by PTLC to give (4aR,7R,8S,8aR) 2-amino-9-(8-fluoro-2-phenyl-hexahydropyrano[3,2-d][1,3]dioxin-7-yl)-1,9-dihydro-purin-6-one.

Yield: 10.8 mg (73%); R_{f}=0.5 (10% MeOH—CH₂Cl₂). ¹H NMR (acetone-d₆, 300 MHz) δ 3.87-4.04 (2H, m), 4.34-4.48 (3H, m), 4.70 (1H, m), 5.10-5.26 (1H, d), 5.75 (1H, s), 6.25 (2H, br), 7.34-7.47 (5H, m), 7.83 (1H, s).

### Example 12

### Preparation of (2R,3R,4S,5R) 2-amino-9-(4-fluoro-5-hydroxy-6-hydroxymethyl-tetrahydropyran-3-yl)-1,9-dihydro-purin-6-one

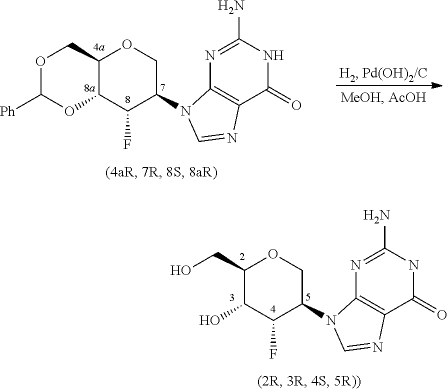

To a solution of (4aR,7R,8S,8aR) 2-amino-9-(8-fluoro-2-phenyl-hexahydropyrano[3,2-d][1,3]dioxin-7-yl)-1,9-dihydro-purin-6-one (2.6 mg, 0.0067 mmol) in 3 mL of MeOH was added 3 drops of acetic acid and 10 mg of Pearlman's catalyst. The mixture was stirred under hydrogen (1 atm) for 1 hour, filtered through a plug of cotton wool, and concentrated in vacuo to give (2R,3R,4S,5R) 2-amino-9-(4-fluoro-5-hydroxy-6-hydroxymethyl-tetrahydropyran-3-yl)-1,9-dihydro-purin-6-one.

Yield: 1.8 mg (90%); R_{f}=0.05 (10% MeOH—CH₂Cl₂). ¹H NMR (CD3OD, 300 MHz) δ 3.65-3.90 (4H, m), 4.18-4.30 (2H, m), 4.76 (1H, m), 4.94-5.11 (1H, d), 8.11 (1H, s).

### Example 13

### Preparation of 3-(2-trimethylsilylethoxymethyl)-1H-pyrimidine-2,4-dione

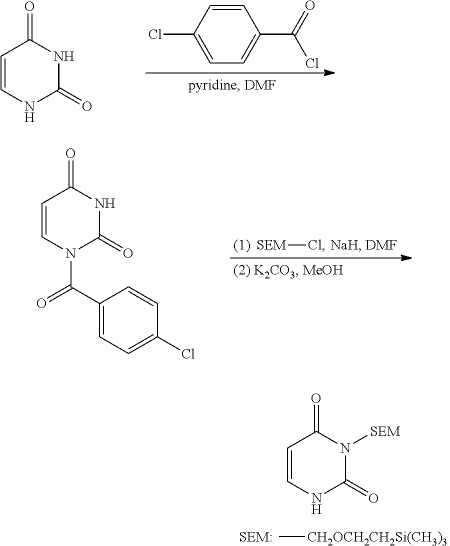

To a suspension of uracil (3 g, 0.027 mol) in 20 mL of DMF was added 4.3 mL of pyridine and 4-chloro-benzoyl chloride (3.7 mL, 0.029 mol). The mixture was stirred at room temperature overnight, and filtered. The residue was washed with CH₃CN and dried in vacuo to give 1-(4-chlorobenzoyl)-1H-pyrimidine-2,4-dione.

Yield: 5.13 (76%). ¹H NMR (CDCl3, 300 MHz) δ 5.95 (1H, d), 7.46 (2H, d), 7.70 (2H, d), 7.82 (1H, d).

To a suspension of 1-(4-chloro-benzoyl)-1H-pyrimidine-2,4-dione (2 g, 8.0 mmol) in 20 mL of DMF was added NaH (240 mg, 10 mmol) and SEM chloride (1.7 mL, 9.6 mmol) at 0° C. The mixture was stirred at room temperature for 2 hours. After aqueous work-up and extraction with Et₂O, the combined organic fractions were dried over Na₂SO₄, filtered and concentrated in vacuo. The residue was dissolved in 20 mL of MeOH and K₂CO₃ (3 g, 21.7 mmol) was added. The mixture was stirred at room temperature for 2 hours, and was concentrated in vacuo. Saturated aqueous NH₄Cl solution was added, and the product was extracted with EtOAc. The combined organic fractions were dried over Na₂SO₄, filtered, and concentrated in vacuo. The crude product was purified by flash chromatography to give 3-(2-trimethylsilylethoxymethyl)-1H-pyrimidine-2,4-dione.

Yield: 1.55 g (80%); R_{f}=0.35 (1:1 EtOAc-hexane). ¹H NMR (CDCl₃, 300 MHz) δ 0.01 (9H, s), 0.99 (2H, m), 3.70 (2H, m), 5.29 (2H, s), 5.79 (1H, dd), 7.21 (1H, dd), 10.24 (1H, d).

### Example 14

### Preparation of 5-methyl-3-(2-trimethylsilylethoxymethyl)-1H-pyrimidine-2,4-dione

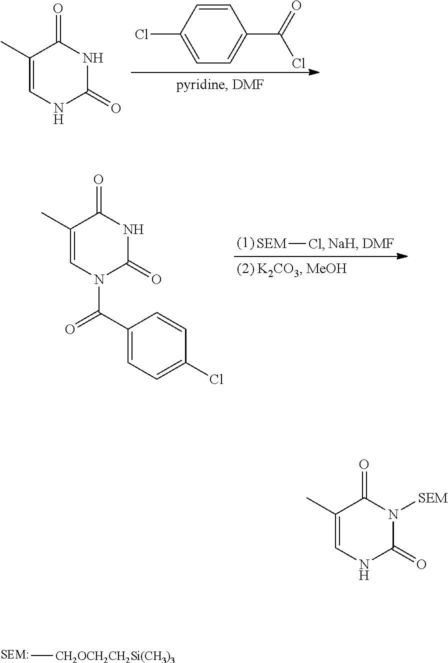

Following the procedure described above, 5 g of 5-methyl-1H-pyrimidine-2,4-dione was converted to 7.72 g (74% yield) of 5-methyl-1-(4-chlorobenzoyl)-1H-pyrimidine-2,4-dione. Similarly, 2.5 g of 5-methyl-1-(4-chloro-benzoyl)-1H-pyrimidine-2,4-dione was converted to 2.10 g of 5-methyl-3-(2-trimethylsilylethoxymethyl)-1H-pyrimidine-2,4-dione.

Yield: 2.10 g (87%); R_{f}=0.35 (1:1 EtOAc-hexane). ¹H NMR (CDCl₃, 300 MHz) δ 0.06 (9H, s), 0.91 (2H, m), 1.86 (3H, s), 3.63 (2H, m), 5.34 (2H, s), 7.06 (1H, d), 10.33 (1H, d).

### Example 15

### Preparation of 5-iodo-3-(2-trimethylsilylethoxymethyl)-1H-pyrimidine-2,4-dione

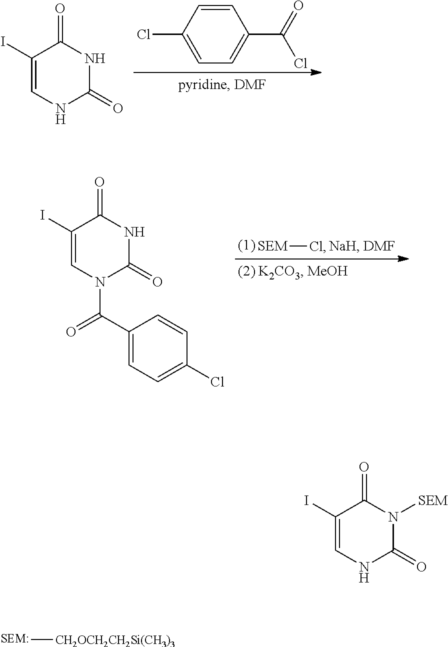

Following the procedure described above, 5 g of 5-iodo-1H-pyrimidine-2,4-dione was converted to 3.58 g of 5-iodo-3-(2-trimethylsilylethoxymethyl)-1H-pyrimidine-2,4-dione.

Yield: 3.58 g (46% overall yield); R_{f}=0.6 (1:1 EtOAc-hexane). ¹H NMR (CDCl₃, 300 MHz) δ 0.03 (9H, s), 0.99 (2H, t), 3.71 (2H, t), 5.44 (2H, s), 7.67 (1H, d), 9.94 (1H, d).

### Example 16

### Preparation of (4aR,7R,8S,8aR) 1-(8-fluoro-2-phenyl-hexahydropyrano[3,2-d][1,3]-dioxin-7-yl)-3-(2-trimethylsilylethoxymethyl)-1H-pyrimidine-2,4-dione

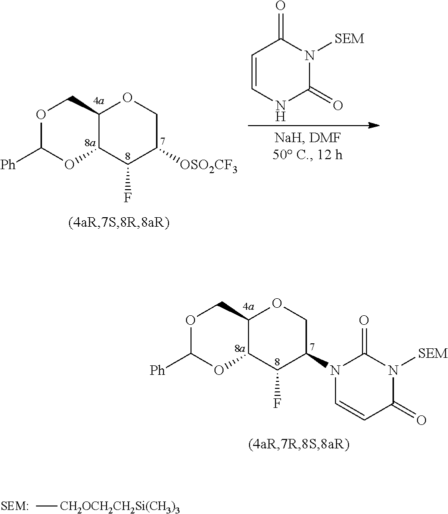

To a solution of (4aR,7S,8R,8aR) trifluoromethanesulfonic acid 8-fluoro-2-phenyl-hexahydropyrano[3,2-d][1,3]dioxin-7-yl ester (64.6 mg, 0.167 mmol) in 5 mL of DMF was added 3-(2-trimethylsilylethoxymethyl)-1H-pyrimidine-2,4-dione (610 mg, 2.5 mmol) and NaH (60 mg, 2.5 mmol). The mixture was stirred at 50° C. for 12 hours. After aqueous work-up and extraction with Et₂O, the combined organic fractions were dried over Na₂SO₄, filtered, and concentrated in vacuo. The crude product was purified by flash chromatography to give (4aR,7R,8S,8aR) 1-(8-fluoro-2-phenyl-hexahydropyrano[3,2-d][1,3]-dioxin-7-yl)-3-(2-trimethylsilylethoxymethyl)-1H-pyrimidine-2,4-dione.

Yield: 36.7 mg (46% yield); R_{f}=0.4 (1:1 EtOAc-hexane). ¹H NMR (CDCl₃, 300 MHz) δ 0.02 (9H, s), 0.99 (2H, m), 3.62-3.79 (4H, m), 4.04-4.17 (2H, m), 4.33-4.44 (2H, m), 4.74 (1H, m), 5.00-5.16 (1H, d), 5.40 (2H, s), 5.58 (1H, s), 5.85 (1H, d), 7.35-7.48 (5H, m), 7.95 (1H, d).

### Example 17

### Preparation of (2R,3R,4S,5R) 1-(4-fluoro-5-hydroxy-6-hydroxymethyl-tetrahydropyran-3-yl)-1H-pyrimidine-2,4-dione

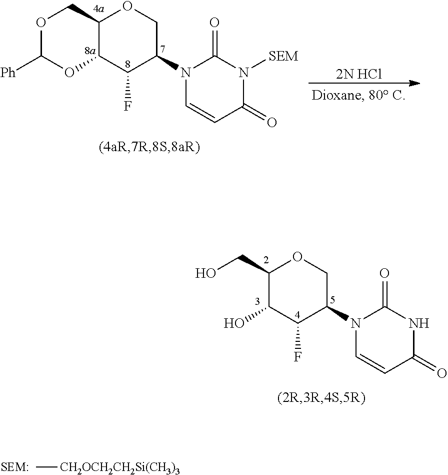

To a solution of (4aR,7R,8S,8aR) 1-(8-fluoro-2-phenyl-hexahydropyrano[3,2-d][1,3]-dioxin-7-yl)-3-(2-trimethylsilylethoxymethyl)-1H-pyrimidine-2,4-dione (19.6 mg, 0.041 mmol) in 3 mL of dioxane was added 3 mL of 2N HCl. The mixture was stirred at 80° C. overnight. The product was extracted with 100 mL of EtOAc. The organic fraction was dried over Na₂SO₄, filtered, and concentrated in vacuo. The crude product was purified by PTLC purification to give (2R,3R,4S,5R) 1-(4-fluoro-5-hydroxy-6-hydroxymethyl-tetrahydropyran-3-yl)-1H-pyrimidine-2,4-dione.

Yield: 3.5 mg (33% yield); R_{f}=0.25 (10% MeOH—CH₂Cl₂). ¹H NMR (acetone-d₆, 300 MHz) δ 3.70 (1H, m), 3.82 (2H, m), 3.91-4.02 (1H, ddd), 4.11-4.24 (2H, m), 4.67 (1H, m), 4.90-5.06 (1H, dt), 5.56 (1H, d), 8.12 (1H, d).

### Example 18

### Preparation of (4aR,7R,8S,8aR) 1-(8-fluoro-2-phenyl-hexahydropyrano[3,2-d][1,3]dioxin-7-yl)-5-methyl-3-(2-trimethylsilylethoxymethyl)-1H-pyrimidine-2,4-dione

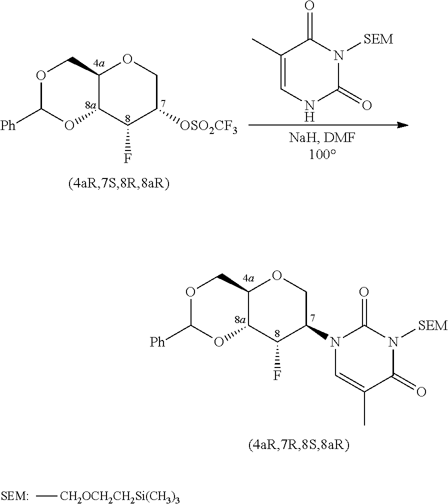

To a solution of (4aR,7S,8R,8aR) trifluoromethanesulfonic acid 8-fluoro-2-phenyl-hexahydropyrano[3,2-d][1,3]dioxin-7-yl ester (41 mg, 0.16 mmol) in 10 mL of DMF was added 5-methyl-3-(2-trimethylsilylethoxymethyl)-1H-pyrimidine-2,4-dione (1.21 g, 4.7 mmol) and NaH (113 mg, 4.7 mmol). The mixture was stirred at 100° C. for 24 hours. After aqueous work-up and extraction with Et₂O, the combined organic fractions were dried over Na₂SO₄, filtered and concentrated in vacuo. The crude product was purified by flash chromatography to give (4aR,7R,8S,8aR) 1-(8-fluoro-2-phenyl-hexahydropyrano[3,2-d][1,3]dioxin-7-yl)-5-methyl-3-(2-trimethylsilylethoxymethyl)-1H-pyrimidine-2,4-dione. (4aR,7S,8R,8aR) trifluoromethanesulfonic acid 8-fluoro-2-phenyl-hexahydropyrano[3,2-d][1,3]dioxin-7-yl ester was also recovered (12.9 mg, 31%).

Yield: 10.1 mg (21%); R_{f}=0.4 (1:1 EtOAc-hexane). ¹H NMR (CDCl₃, 300 MHz) δ 0.02 (9H, s), 1.00 (2H, m), 2.02 (3H, d), 3.63-3.81 (4H, m), 4.05-4.18 (2H, m), 4.32-4.45 (2H, m), 4.74 (1H, m), 4.99-5.15 (1H, d), 5.42 (2H, s), 5.60 (1H, s), 7.35-7.49 (5H, m), 7.78 (1H, m).

### Example 19

### Preparation of (2R,3R,4S,5R) 1-(4-fluoro-5-hydroxy-6-hydroxymethyl-tetrahydropyran-3-yl)-5-methyl-1H-pyrimidine-2,4-dione

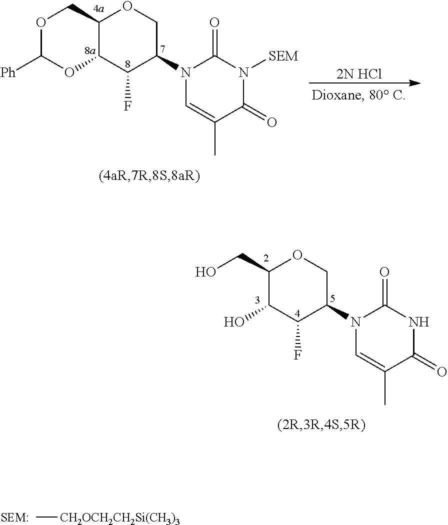

To a solution of (4aR,7R,8S,8aR) 1-(8-fluoro-2-phenyl-hexahydropyrano[3,2-d][1,3]dioxin-7-yl)-5-methyl-3-(2-trimethylsilylethoxymethyl)-1H-pyrimidine-2,4-dione (6.0 mg, 0.012 mmol) in 3 mL of dioxane was added 3 mL of 2N HCl. The mixture was stirred at 80° C. overnight. The product was extracted with 100 mL of EtOAc. The organic fraction was dried over Na₂SO₄, filtered and concentrated in vacuo. The crude product was purified by PTLC to give (2R,3R,4S,5R) 1-(4-fluoro-5-hydroxy-6-hydroxymethyl-tetrahydropyran-3-yl)-5-methyl-1H-pyrimidine-2,4-dione.

Yield: 2.0 mg (61%); R_{f}=0.25 (10% MeOH—CH₂Cl₂). ¹H NMR (acetone-d₆, 300 MHz) δ 1.83 (3H, d), 3.68 (1H, m), 3.83 (2H, d), 3.94-4.03 (1H, ddd), 4.09-4.21 (2H, m), 4.45 (1H, br), 4.68 (1H, m), 4.88-5.04 (1H, dt), 7.97 (1H, m), 10.03 (1H, br).

### Example 20

### Preparation of (4aR,7R,8S,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione

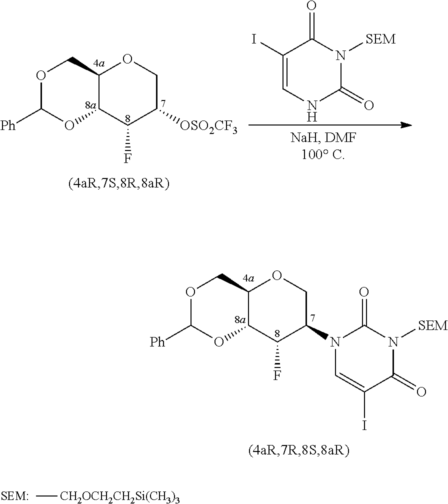

To a solution of (4aR,7S,8R,8aR) trifluoromethanesulfonic acid 8-fluoro-2-phenyl-hexahydropyrano[3,2-d][1,3]dioxin-7-yl ester (23.7 mg, 0.061 mmol) in 4 mL of DMF was added 5-iodo-3-(2-trimethylsilylethoxymethyl)-1H-pyrimidine-2,4-dione (400 mg, 1.1 mmol) and NaH (26 mg, 1.1 mmol). The mixture was stirred at 80° C. overnight. After aqueous work-up and extraction with Et₂O, the product was purified by chromatography to give (4aR,7R,8S,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione.

Yield: 19.4 mg (52%); R_{f}=0.35 (1:3 EtOAc-hexane). ¹H NMR (CDCl₃, 300 MHz) δ 0.02 (9H, s), 0.99 (2H, m), 3.61-3.82 (4H, m), 4.05-4.21 (2H, m), 4.34-4.46 (2H, m), 4.73 (1H, m), 4.98-5.14 (1H, d), 5.46 (2H, s), 5.62 (1H, s), 7.36-7.49 (5H, m), 8.41 (1H, s).

### Example 21

### Preparation of (2R,3R,4S,5R) 1-(4-fluoro-5-hydroxy-6-hydroxymethyl-tetrahydropyran-3-yl)-5-iodo-1H-pyrimidine-2,4-dione

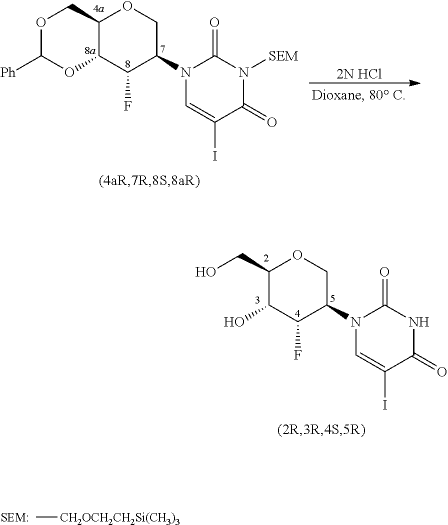

To a solution of (4aR,7R,8S,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione (19.4 mg, 0.032 mmol) in 3 mL of dioxane was added 3 mL of 2N HCl. The mixture was stirred at 80° C. overnight. The product was extracted with 100 mL of EtOAc. The organic fraction was dried over Na₂SO₄, filtered and concentrated in vacuo. The crude product was purified by PTLC to give (2R,3R,4S,5R) 1-(4-fluoro-5-hydroxy-6-hydroxymethyl-tetrahydropyran-3-yl)-5-iodo-1H-pyrimidine-2,4-dione.

Yield: 6.0 mg (49%); R_{f}=0.25 (10% MeOH—CH₂Cl₂). ¹H NMR (acetone-d₆, 300 MHz) δ 3.70 (1H, m), 3.84 (2H, m), 3.91-4.07 (2H, m), 4.12-4.29 (2H, m), 4.46 (1H, d), 4.71 (1H, m), 4.92-5.09 (1H, dt), 8.55 (1H, s), 10.45 (1H, br).

### Example 22

### Preparation of 5,6-dichloro-1H-benzoimidazole

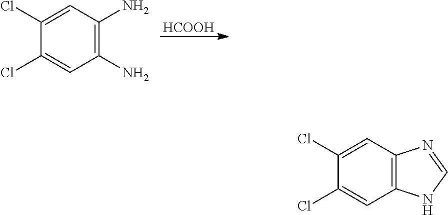

4,5-Dichlorobenzene-1,2-diamine (500 mg, 2.82 mmol) was dissolved in 10 mL of HCOOH and stirred under reflux for 1 hour. The solvent was removed in vacuo, the residue was dissolved in EtOAc and NEt₃ (3 drops) was added. The solvent was removed in vacuo and the crude product was purified by flash chromatography to give 5,6-dichloro-1H-benzoimidazole.

Yield: 480 mg (91%); R_{f}=0.5 (10% MeOH—CH₂Cl₂). ¹H NMR (acetone-d₆, 300 MHz) δ 7.83 (2H, s), 8.29 (1H, s), 11.8 (1H, br).

### Example 23

### Preparation of (4aR,7R,8S,8aR) 5,6-dichloro-1-(8-fluoro-2-phenyl-hexahydro-pyrano[3,2-d][1,3]dioxin-7-yl)-1H-benzoimidazole

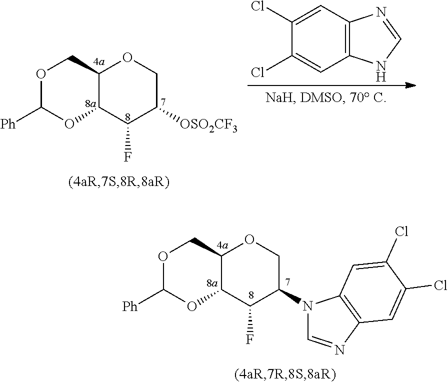

To a solution of (4aR,7S,8R,8aR) trifluoromethanesulfonic acid 8-fluoro-2-phenyl-hexahydropyrano[3,2-d][1,3]dioxin-7-yl ester (47.2 mg, 0.12 mmol), prepared according to earlier procedures, in 5 mL of DMSO was added 5,6-dichloro-1H-benzoimidazole (524 mg, 2.8 mmol) and NaH (67 mg, 2.8 mmol). The mixture was stirred at 70° C. overnight. After aqueous work-up and extraction with Et₂O, The combined organic fractions were washed with brine, dried over Na₂SO₄, filtered, and concentrated in vacuo. The crude product was purified by flash chromatography to give (4aR,7R,8S,8aR) 5,6-dichloro-1-(8-fluoro-2-phenyl-hexahydro-pyrano[3,2-d][1,3]dioxin-7-yl)-1H-benzoimidazole.

Yield: 29.4 mg (58%); R_{f}=0.5 (1:1 EtOAc-hexane). ¹H NMR (CDCl₃, 300 MHz) δ 3.61-3.75 (1H, ddd), 3.80 (1H, dt), 4.14 (1H, m), 4.44-4.51 (3H, m), 4.66 (1H, m), 5.05-5.21 (1H, d), 5.49 (1H, s), 7.26-7.44 (5H, m), 7.60 (1H, s), 7.97 (1H, s), 8.51 (1H, s).

### Example 24

### Preparation of (2R,3R,4S,5R) 5-(5,6-dichlorobenzoimidazol-1-yl)-4-fluoro-2-hydroxymethyl-tetrahydropyran-3-ol

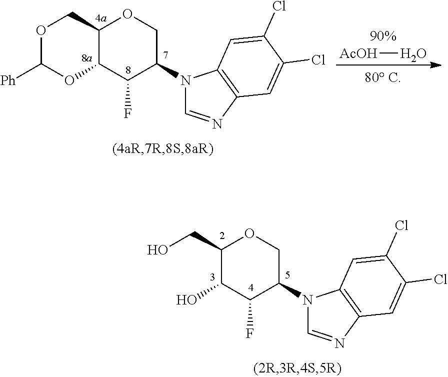

Compound (2R,3R,4S,5R) 5-(5,6-dichloro-benzoimidazol-1-yl)-4-fluoro-2-hydroxymethyl-tetrahydropyran-3-ol (5.1 mg, 0.012 mmol) was dissolved in 3 mL of 9:1 acetic acid-water. The mixture was stirred at 80° C. for 1 hour and was concentrated in vacuo. Acetone (2 mL) was added, followed by 3 drops of NEt₃. The solvent was removed in vacuo, and the crude product was purified by PTLC to give (2R,3R,4S,5R) 5-(5,6-dichlorobenzoimidazol-1-yl)-4-fluoro-2-hydroxymethyl-tetrahydropyran-3-ol.

Yield: 2.8 mg (70%); R_{f}=0.4 (10% MeOH—CH₂Cl₂). ¹H NMR (acetone-d₆, 300 MHz) δ 3.78 (1H, m), 3.87-3.91 (2H, m), 3.97-4.06 (1H, m), 4.27-4.42 (2H, m), 4.50 (1H, d), 4.99 (1H, m), 5.04-5.20 (1H, dt), 7.87 (1H, s), 8.05 (1H, s), 8.59 (1H, s).

### Example 25

### Preparation of (4aR,7S,8S,8aS) 2-phenyl-hexahydropyrano[3,2-d][1,3]dioxine-7,8-diol

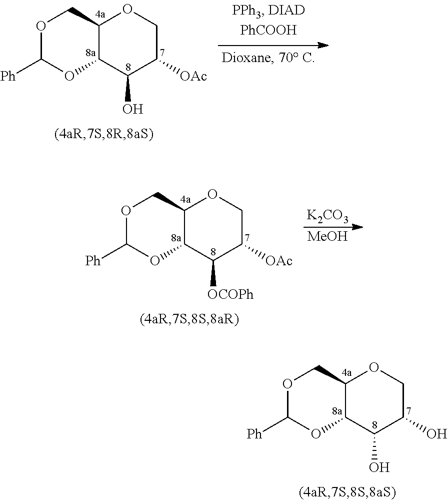

To a solution of (4aR,7S,8R,8aS) acetic acid 8-hydroxy-2-phenyl-hexahydropyrano[3,2-d][1,3]dioxin-7-yl ester (1 g, 3.4 mmol) in 40 mL of dioxane was added PPh₃ (5.4 g, 20.6 mmol) and benzoic acid (20.5 mmol). To the mixture at 0° C. was slowly added DIAD (diisopropyl azodicarboxylate) (4 mL, 20.3 mmol). The mixture was stirred at 70° C. overnight, concentrated in vacuo. The crude (4aR,7S,8S,8aR) benzoic acid 7-acetoxy-2-phenyl-hexahydropyrano[3,2-d][1,3]dioxin-8-yl ester (R_{f}=0.5 in 3:1 hexane-EtOAc) was dissolved in 36 mL of MeOH and 4 mL of THF. K₂CO₃ (100 mg, 0.72 mmol) was added and the mixture was stirred at room temperature for 8 hours. The solvent was removed in vacuo, and the crude product was purified by chromatography to give (4aR,7S,8S,8aS) 2-phenyl-hexahydropyrano[3,2-d][1,3]dioxine-7,8-diol.

Yield: 505.8 mg (59% yield, 2 steps); R_{f}=0.3 (1:1 EtOAc-hexane). ¹H NMR (CDCl₃, 300 MHz) δ 3.50-3.58 (2H, m), 3.67 (1H, t), 3.77-3.87 (3H, m), 4.33 (1H, m), 4.37 (1H, dd), 5.58 (1H, s), 7.36-7.51 (5H, m).

### Example 26

### Preparation of (4aR,7S,8S,8aS) acetic acid 8-hydroxy-2-phenyl-hexahydropyrano[3,2-d][1,3]dioxin-7-yl ester

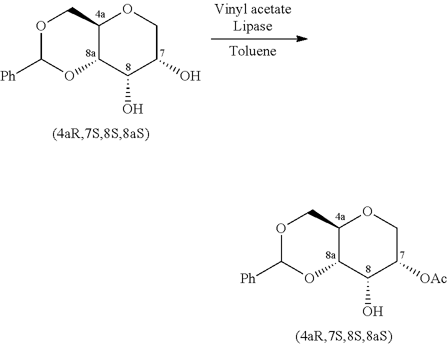

To (4aR,7S,8S,8aS) 2-phenyl-hexahydropyrano[3,2-d][1,3]dioxine-7,8-diol (129.5 mg, 0.514 mmol) in 6 mL of toluene was added lipase PS—I “Amano” I (400 mg) and vinyl acetate (0.4 mL, 4.34 mmol). The mixture was stirred at room temperature for 6 hours, filtered, concentrated in vacuo and purified by chromatography to give (4aR,7S,8S,8aS) acetic acid 8-hydroxy-2-phenyl-hexahydropyrano[3,2-d][1,3]dioxin-7-yl ester.

Yield: 143 mg (95%); R_{f}=0.55 (1:1 EtOAc-hexane). ¹H NMR (CDCl₃, 300 MHz) δ 2.14 (3H, s), 3.61 (1H, dd), 3.67 (1H, t), 3.76-3.84 (2H, m), 3.91 (1H, m), 4.38 (1H, m), 4.46 (1H, m), 4.96 (1H, m), 5.59 (1H, s), 7.37-7.51 (5H, m).

### Example 27

### Preparation of (4aR,7S,8R,8aR) acetic acid 8-fluoro-2-phenyl-hexahydropyrano[3,2-d][1,3]dioxin-7-yl ester

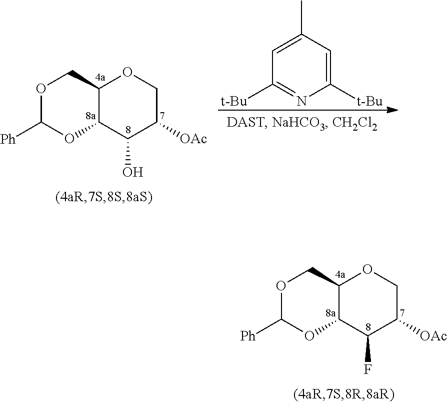

To a solution of (4aR,7S,8S,8aS) acetic acid 8-hydroxy-2-phenyl-hexahydropyrano[3,2-d][1,3]dioxin-7-yl ester (100.0 mg, 0.34 mmol) in 4 mL of CH₂Cl₂ were added 4-methyl-2,6-di-tent-butylpyridine (140 mg, 0.68 mmol), NaHCO₃ (100 mg, 1 mmol) and diethylaminosulfur trifluoride (DAST) (0.045 mL, 0.34 mmol). The mixture was stirred at room temperature for 5 minutes and purified by PTLC to give (4aR,7S,8R,8aR) acetic acid 8-fluoro-2-phenyl-hexahydropyrano[3,2-d][1,3]dioxin-7-yl ester.

Yield: 45.9 mg (46%); R_{f}=0.45 (1:3 EtOAc-hexane). ¹H NMR (CDCl₃, 300 MHz) δ 2.14 (3H, s), 3.31 (1H, t), 3.42 (1H, m), 3.77 (2H, m), 4.19 (1H, m), 4.39 (1H, m), 4.62-4.80 (1H, dt), 5.15 (1H, m), 5.77 (1H, s), 7.36-7.52 (5H, m).

### Example 28

### Preparation of (4aR,7S,8R,8aR) 8-fluoro-2-phenyl-hexahydropyrano[3,2-d][1,3]dioxin-7-ol

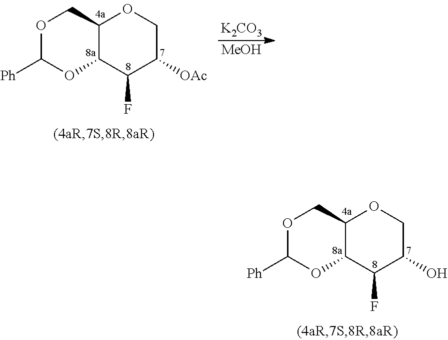

To a solution of (4aR,7S,8R,8aR) acetic acid 8-fluoro-2-phenyl-hexahydropyrano[3,2-d][1,3]dioxin-7-yl ester (45.9 mg, 0.155 mmol) in 5 mL of MeOH was added K₂CO₃ (10 mg, 0.072 mmol). The mixture was stirred at room temperature for 1 hour. The solvent was removed in vacuo, saturated aqueous NH₄Cl was added and the product was extracted with CH₂Cl₂. The combined organic fractions were dried over Na₂SO₄, filtered and concentrated in vacuo. The crude product was purified by flash chromatography to give (4aR,7S,8R,8aR) 8-fluoro-2-phenyl-hexahydropyrano[3,2-d][1,3]dioxin-7-ol.

Yield: 39.1 mg (99%); R_{f}=0.5 (1:1 EtOAc-hexane). ¹H NMR (CDCl₃, 300 MHz) δ 3.45-3.46 (2H, m), 3.71 (1H, q), 3.74 (1H, t), 4.02 (1H, m), 4.10 (1H, m), 4.37 (1H, ddd), 4.48-4.66 (1H, dt), 5.55 (1H, s), 7.36-7.52 (5H, m).

### Example 29

### Preparation of (4aR,7S,8S,8aR) trifluoromethanesulfonic acid 8-fluoro-2-phenyl-hexahydropyrano[3,2-d][1,3]dioxin-7-yl ester

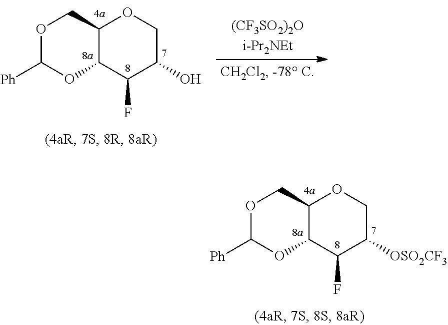

To a solution of 4aR,7S,8R,8aR) 8-fluoro-2-phenyl-hexahydropyrano[3,2-d][1,3]dioxin-7-ol (39.1 mg, 0.154 mmol) in 4 mL of CH₂Cl₂ was added i-Pr₂NEt (0.3 mL, 1.72 mmol), followed by trifluoromethanesulfonic anhydride (0.12 mL, 0.71 mmol) at −78° C. The mixture was stirred at −78° C. for 15 minutes and purified by chromatography to give (4aR,7S,8S,8aR) trifluoromethanesulfonic acid 8-fluoro-2-phenyl-hexahydropyrano[3,2-d][1,3]dioxin-7-yl ester.

Yield: 39.3 mg (66%); R_{f}=0.7 (1:3 EtOAc-hexane) ¹H NMR (CDCl₃, 300 MHz) δ 3.42 (1H, m), 3.58 (1H, t), 3.75-3.85 (2H, m), 4.31 (1H, m), 4.40 (1H, m), 4.71-4.89 (1H, dt), 4.97 (1H, m), 5.57 (1H, s), 7.38-7.51 (5H, m).

### Example 30

### Preparation of (4aR,7R,8R,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione

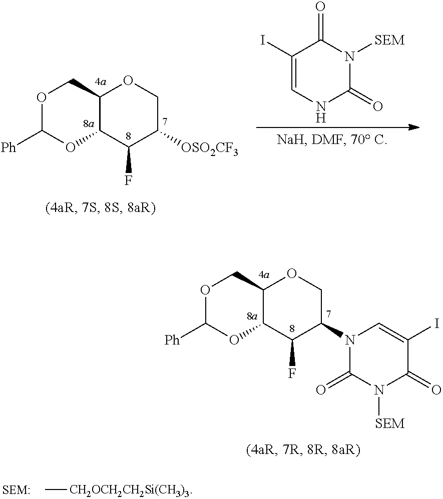

To a solution of (4aR,7S,8S,8aR) trifluoromethanesulfonic acid 8-fluoro-2-phenyl-hexahydropyrano[3,2-d][1,3]dioxin-7-yl ester (39.3 mg, 0.102 mmol) in 4 mL of DMF was added 5-iodo-3-(2-trimethylsilylethoxymethyl)-1H-pyrimidine-2,4-dione (500 mg, 1.4 mmol) and NaH (33 mg, 1.4 mmol). The mixture was stirred at 70° C. overnight. After aqueous work-up and extraction with Et₂O, the combined organic fractions were dried over Na₂SO₄, filtered and concentrated in vacuo. The crude product was purified by flash chromatography to give (4aR,7R,8R,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione.

Yield: 37.0 mg (60%); R_{f}=0.3 (1:3 EtOAc-hexane). ¹H NMR (CDCl₃, 300 MHz) δ 0.02 (9H, s), 0.98 (2H, m), 3.45 (1H, m), 3.69-3.77 (2H, m), 3.84 (1H, t), 4.26-4.39 (3H, m), 4.79-4.97 (1H, ddd), 5.55 (1H, d), 5.62 (1H, d), 5.64 (1H, s), 5.75 (1H, m), 7.36-7.51 (5H, m), 8.06 (1H, s).

### Example 31

### Preparation of (3R,4R,5R,6R) 1-(4-fluoro-5-hydroxy-6-hydroxymethyl-tetrahydropyran-3-yl)-5-iodo-1H-pyrimidine-2,4-dione

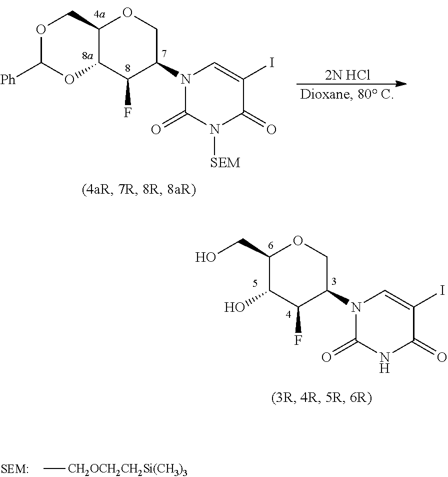

To a solution of (4aR,7R,8R,8aR) 1-(8-fluoro-2-phenyl-hexahydropyrano[3,2-d][1,3]dioxin-7-yl)-5-iodo-3-(2-trimethylsilylethoxymethyl)-1H-pyrimidine-2,4-dione (9.8 mg, 0.016 mmol) in 3 mL of dioxane solution was added 3 mL of 2N HCl. The mixture was stirred at 80° C. for 6 hours. The product was extracted with 100 mL of EtOAc. The organic fraction was dried over Na₂SO₄, filtered, concentrated in vacuo, and purified by PTLC to give (3R,4R,5R,6R) 1-(4-fluoro-5-hydroxy-6-hydroxymethyl-tetrahydropyran-3-yl)-5-iodo-1H-pyrimidine-2,4-dione.

Yield: 3.9 mg (60%); R_{f}=0.2 (10% MeOH—CH₂Cl₂). ¹H NMR (Acetone-d₆, 300 MHz) δ 3.38 (1H, m), 3.89 (2H, m), 4.05-4.12 (2H, m), 4.36 (1H, ddd), 4.82-4.98 (1H, ddd), 5.28 (1H, m), 8.76 (1H, s).

### Example 32

### Preparation of (4aR,7R,8R,8aR) 4-amino-1-(8-fluoro-2-phenyl-hexahydro-pyrano[3,2-d][1,3]dioxin-7-yl)-1H-pyrimidin-2-one

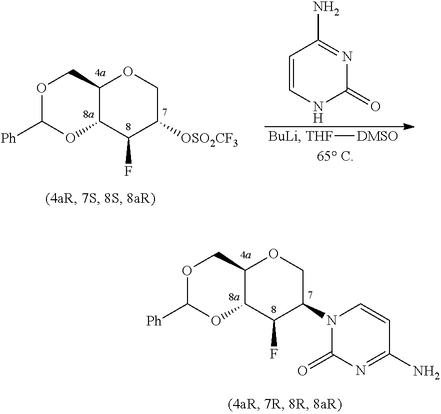

To a suspension of cytosine (450 mg, 4.05 mmol) in 8 mL of THF was added n-BuLi (1.46 mL of a 2.5 M solution in hexane, 3.65 mmol) at 0° C. DMSO (8 mL) was added at 0° C. and the mixture was stirred at room temperature to give a clear solution that was added to (4aR,7S,8S,8aR) trifluoromethanesulfonic acid 8-fluoro-2-phenyl-hexahydropyrano[3,2-d][1,3]dioxin-7-yl ester (24.5 mg, 0.063 mmol). The mixture was stirred at 70° C. overnight. After aqueous work-up and extraction with EtOAc, the combined organic fractions were dried over Na₂SO₄, filtered, and concentrated in vacuo. The crude product was purified by flash chromatography to give (4aR,7R,8R,8aR) 4-amino-1-(8-fluoro-2-phenyl-hexahydro-pyrano[3,2-d][1,3]dioxin-7-yl)-1H-pyrimidin-2-one.

Yield: 12.2 mg (56%); R_{f}=0.5 (EtOAc). ¹H NMR (CDCl₃, 300 MHz) δ 3.45 (1H, m), 3.65-3.74 (2H, m), 3.89 (1H, t), 4.32 (1H, m), 4.47 (1H, q), 4.77-4.93 (1H, ddd), 5.17 (2H, br s), 5.65 (1H, s), 5.72 (1H, br s), 6.18 (1H, d), 7.35-7.52 (5H, m), 7.99 (1H, d).

### Example 33

### Preparation of (3R,4R,5R,6R) 4-amino-1-(4-fluoro-5-hydroxy-6-hydroxymethyl-tetrahydropyran-3-yl)-1H-pyrimidin-2-one

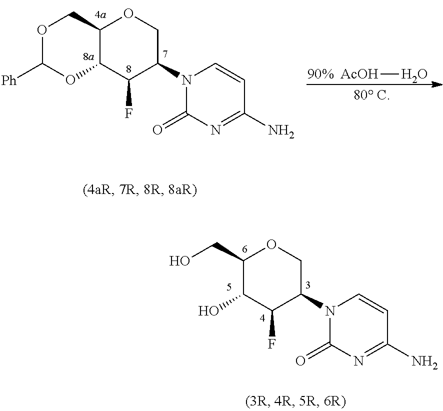

(4aR,7R,8R,8aR) 4-amino-1-(8-fluoro-2-phenyl-hexahydro-pyrano[3,2-d][1,3]dioxin-7-yl)-1H-pyrimidin-2-one (12.2 mg, 0.035 mmol) was dissolved in 3 mL of 9:1 acetic acid-water. The mixture was stirred at 80° C. for 1 hour, concentrated in vacuo and purified by PTLC to give (3R,4R,5R,6R) 4-amino-1-(4-fluoro-5-hydroxy-6-hydroxymethyl-tetrahydropyran-3-yl)-1H-pyrimidin-2-one.

Yield: 3.1 mg (34%); R_{f}=0.15 (10% MeOH—CH₂Cl₂). ¹H NMR (Acetone-d₆, 300 MHz) δ 3.27 (1H, m), 3.63-3.75 (2H, m), 3.89 (1H, d), 4.07-4.17 (2H, m), 4.52-4.55 (1H, ddd), 4.8 (1H, br), 5.54 (1H, m), 6.20 (1H, d), 6.27 (2H, br), 7.89 (1H, d).

### Example 34

### Preparation of allyloxy-acetic acid ethyl ester

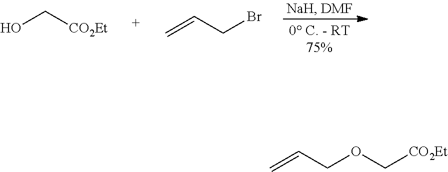

A round-bottom flask was charged with NaH (1.76 g, 44 mmol, 60% dispersion in mineral oil) and flushed with argon. Hexane-(10 ml×2) was added and decanted. DMF (10 ml) was added into the flask and the resulting solution was cooled to 0° C. Ethyl glycolate (4.16 g, 40.0 mmol) was added over 10 min. The solution was allowed to gradually warm to 25° C. and was maintained at that temperature for 2 h. The solution was cooled to 0° C. and allyl bromide (5.32 g, 44.0 mmol) was added over 10 min. The solution was allowed to gradually warm to 25° C. and stirred at that temperature for 2 h. Aqueous solution NH₄Cl (10 ml) was added to the reaction and the mixture was diluted with EtOAc (60 ml). The organic layer was separated and washed with H₂O (20 ml×²), dried over Na₂SO₄ and concentrated in vacuo. The crude product was purified by distillation under reduced pressure.

Yield=4.29 g, 75%; colorless liquid; bp=38-39° C., 2 mmHg. IR (neat): 1985, 1756, 1724, 1203, 1130 cm⁻¹. ¹H NMR (CDCl₃, 400 MHz): δ 5.90-5.70 (m, 1H), 5.25-5.00 (m, 2H), 4.10-4.20 (m, 2H), 3.92-4.05 (m, 4H), 1.21 (t, J=7 Hz, 3H). ¹³C NMR (CDCl₃, 100 MHz): δ 170.09 (C), 133.62 (CH), 117.78 (CH₂), 72.10 (CH₂), 66.99 (CH₂), 60.53 (CH₂), 13:94 (CH₃). MS (m/z, relative intensity): 144 (M⁺, 14), 115 (22), 103 (100), 83 (85); HRMS: calculated for C₇H₁₂O₃ (M⁺): 144.0786. found 144.0783.

### Example 35

### Preparation of 2-allyloxy-3-hydroxypent-4-enoic acid ethyl ester

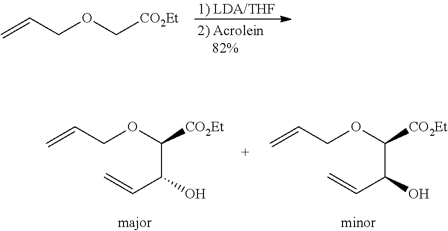

Under an atmosphere of argon, n-BuLi (3 mmol, 1.2 ml, 2.5 M in hexane) was added dropwise to a solution of diisopropylamine (281 mg, 2.78 mmol) in dry THF (20 ml) at −78° C. After stirring for 20-30 min, a solution of allyloxy-acetic acid ethyl ester (200 mg, 1.38 mmol) in THF (4 ml) was added and the mixture was stirred at −78° C. for 10 min. Acrolein (79 mg, 1.38 mmol) was added into the reaction mixture and stirring was maintained until all starting materials were consumed. The reaction was quenched by addition of EtOH (2 ml) and warmed to room temperature. The solution was diluted with EtOAc (60 ml), washed with H₂O (20 ml×2), dried over Na₂SO₄ and concentrated in vacuo. The crude product was purified by flash chromatography with 20% EtOAc-hexane.

Yield=961 mg, 82%; colorless liquid; R_{f}=0.25 in 20% EtOAc-hexane). IR (neat): 3300-3600, 2977, 1742, 1364, 1231, 1134, 1028, 927 cm⁻¹. ¹H NMR (CDCl₃, 400 MHz): δ 6.0-5.8 (m, 2H), 5.3-5.1 (m, 4H), 4.4-4.2 (m, 1H), 4.2-4.0 (m, 3H), 3.9-3.8 (m, 2H), 2.73 (br s, 1H), 1.2 (t, J=7 Hz, 3H). ¹³C NMR (CDCl₃, 100 MHz, 2:1 isomeric forms, * denotes minor isomer): δ 170.35* (C), 170.08 (C), 135.77* (CH), 135.42 (CH), 133.56 (CH), 133.47* (CH), 118.23* (CH₂), 118.12 (CH₂), 117.14* (CH₂), 116.96 (CH₂), 80.85 (one CH and one CH*), 73.32* (CH), 72.99 (CH), 71.88* (CH₂), 71.87 (CH₂), 60.99* (CH₂), 60.91 (CH₂), 14.08 (CH₃), 14.05* (CH₃). MS (m/z, relative intensity): 200 (M⁺, 7), 182 (27), 153 (41), 136 (51), 115 (37), 95 (100); HRMS calculated for C₁₀H₁₆O₄ (M⁺): 200.1048. found 200.1044.

### Example 36

### Preparation of 3-hydroxy-3,6-dihydro-2H-pyran-2-carboxylic acid ethyl ester

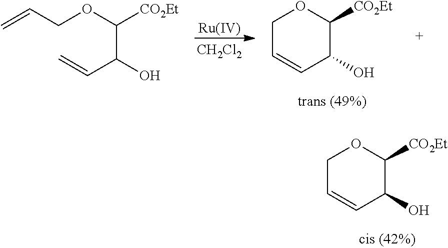

To a solution of 2-allyloxy-3-hydroxypent-4-enoic acid ethyl ester (200 mg, 1.0 mmol) in CH₂Cl₂ (10 ml) was added bis-(tricyclohexylphosphine)-benzylidine ruthenium (IV) chloride (20 mg, 0.024 mmol) and the resulting mixture was stirred at ambient temperature for 4 h. Bis-(tricyclohexylphosphine)-benzylidine ruthenium (IV) chloride (20 mg, 0.024 mmol) was added again and the resulting mixture was stirred at ambient temperature for an additional 10 h. The solution was concentrated in vacuo. The crude product was purified by flash chromatography with 25 to 30% EtOAc-hexane

Trans 3-hydroxy-3,6-dihydro-2H-pyran-2-carboxylic acid ethyl ester: Yield=84 mg (49%); R_{f}=0.29 in 40% EtOAc-hexane. IR (neat): 3200-3600, 2976, 1747, 1185, 1111, 1024 cm⁻¹; ¹H NMR (CDCl₃, 400 MHz): δ 5.78 (br s, 2H), 4.10-4.35 (m, 5H), 3.93 (d, J=7 Hz, 1H), 3.07 (br s, 1H), 1.24 (t, J=7 Hz, 3H). ¹³C NMR (CDCl₃, 100 MHz): δ 170.58 (C), 127.49 (CH), 126.54 (CH), 77.00 (CH), 64.98 (CH₂), 64.32 (CH), 61.53 (CH₂), 14.00 (CH₃). MS (m/z, relative intensity): 172 (M⁺, 2), 141 (6), 112 (16), 81 (100); exact mass calculated for C₈H₁₂O₄ (M⁺): 172.0735. found 172.0733. Cis 3-hydroxy-3,6-dihydro-2H-pyran-2-carboxylic acid ethyl ester:

Yield=69 mg (42%); R_{f}=0.06 in 40% EtOAc-hexane. qIR (neat): 3200-3600, 2975, 2926, 2841, 1746, 1642, 1182, 1097 cm⁻¹. ¹H NMR (CDCl₃, 500 MHz): δ 6.00-6.15 (m, 1H), 5.90-5.96 (m, 1 μl), 4.10-4.40 (m, 7H), 1.28 (t, J=7 Hz, 3H). ¹³C NMR (CDCl₃, 100 MHz): δ 169.07 (C), 130.15 (CH), 125.96 (CH), 77.50 (CH), 66.09 (CH₂), 63.48 (CH), 61.36 (CH₂), 14.21 (CH₃). MS (m/z, relative intensity): 172 (M⁺, 2), 141 (6), 112 (16), 81 (100); exact mass calculated for C₈H₁₂O₄ (M⁺): 172.0735. found 172.0730.

### Example 37

### Resolution of racemic trans-3-hydroxy-3,6-dihydro-2H-pyran-2-carboxylic acid ethyl ester using lipase from Alcaligenes sp

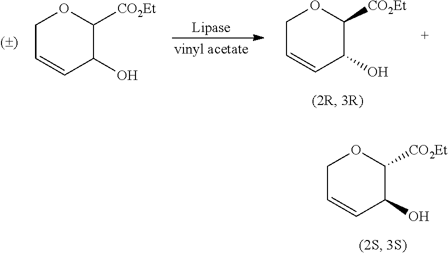

Vinyl acetate (200 μl) was added to a suspension of racemic trans-3-hydroxy-3,6-dihydro-2H-pyran-2-carboxylic acid ethyl ester (53 mg) and immobilized lipase from Alcaligenes sp. (50 mg) in 5 ml of toluene. The mixture was agitated for 14 h at ambient temperature. The mixture was filtered and the filtrate was concentrated in vacuo. The crude product was purified by flash chromatography with 20% EtOAc-hexane.

Trans-(2R,3R)-3-Acetoxy-3,6-dihydro-2H-pyran-2-carboxylic acid ethyl ester: Yield=32 mg (49%); optical purity: >95% ee; [α]_{D}^{27.4}−124.8 (CHCl₃, c 0.31). Trans-(2S,3S)-3-hydroxy-3,6-dihydro-2H-pyran-2-carboxylic acid ethyl ester: Yield=25 mg (47%); optical purity: >99.9% ee; [α]_{D}^{28.3}−14.6 (CHCl₃, c 0.33).

### Example 38

### Preparation of (2R,3S) trans-2-hydroxymethyl-3,6-dihydro-2H-pyran-3-ol

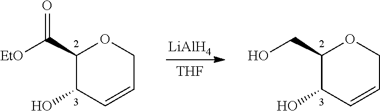

To a solution of (2S,3S) trans-3-hydroxy-3,6-dihydro-2H-pyran-2-carboxylic acid ethyl ester (108 mg, 0.63 mmol) in THF (5 mL) was added LiAlH₄ (84 mg, 2.21 mmol). The resulting solution was stirred at ambient temperature for 15 min. The reaction was completed in ca. 15 min, monitor by TLC. The reaction was quenched by addition of H₂O (10 mL). The solution was diluted with EtOAc (50 mL×2), washed with brine (50 mL), dried over Na₂SO₄, concentrated in vacuo and purified by flash column chromatography with EtOAc (R_{f}=0.25 in EtOAc) to give (2R,3S) trans-2-hydroxymethyl-3,6-dihydro-2H-pyran-3-ol as a colorless liquid (68 mg, 84% yield, 99.9% e.e).

IR (neat): 3100-3600, 2983, 1642, 1376, 1186, 1114, 1038 cm⁻¹. ¹H NMR (CDCl₃, 400 MHz): δ 5.77-5.84 (m, 2H), 4.16-4.19 (m, 3H), 3.88 (dd, J=11.5, 3.9 Hz, 1H), 3.78 (dd, J=11.5, 5.5 Hz, 1H), 3.30-3.33 (m, 1H), 1.77 (br s, 2H). ¹³C NMR (CDCl₃, 100 MHz): δ 128.41 (CH), 127.80 (CH), 78.46 (CH), 65.31 (CH₂), 64.42 (CH), 63.24 (CH₂). MS (m/z, relative intensity): 130 (M⁺, 8), 112 (29), 97 (61), 81 (100); exact mass calculated for C₆H₁₀O₃ (M⁺): 130.0630. found 130.0633. [α]_{D}^{29.3}+3.04 (CHCl₃, c 0.27).

### Example 39

### Preparation of (4aR,8aS) 2-phenyl-4,4a,6,8a-tetrahydro-pyrano[3,2-d][1,3]dioxine

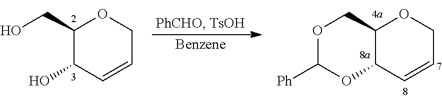

A mixture of (2R,3S) trans-2-hydroxymethyl-3,6-dihydro-2H-pyran-3-ol (458 mg, 3.8 mmol), benzaldehyde (410. mg, 3.8 mmol) and a catalytic amount of p-TsOH (5 mg) in benzene (30 ml) was heated to reflux using a Dean-Stock condenser. After 2 hr, the reaction mixture was cooled and diluted with EtOAc (30 ml), washed with water (10 ml) and brine (10 ml). The residue was purified by flash chromatography (10% EtOAc-hexane) to yield (4aR,8aS) 2-phenyl-4,4a,6,8a-tetrahydro-pyrano[3,2-d][1,3]dioxine.

Colorless solid; yield=604 mg (73%); R_{f}=0.72 in 50% EtOAc-hexane. [α]_{D}^{24.8}=+19.75 (0.15, CHCl₃). ¹H NMR (CDCl₃, 500 MHz): δ 7.47-7.52 (m, 2H), 7.30-7.38 (m, 3H), 5.96 (d, J=10.5 Hz, 1H), 5.75 (d, J=10.5 Hz, 1H), 5.59 (s, 1H), 4.19-4.39 (m, 4H), 3.70-3.80 (m, 1H), 3.50-3.56 (m, 1H). ¹³C NMR (CDCl₃, 125 MHz): δ 137.48 (C), 129.05 (CH), 128.29 (two of CH), 127.48 (CH), 126.20 (two of CH), 126.15 (CH), 101.95 (CH), 75.33 (CH), 70.24 (CH), 69.47 (CH₂), 66.45 (CH₂). MS (m/z, relative intensity): 218 (M⁺, 50), 149 (32), 105 (35), 69 (100).

### Example 40

### Preparation of (4aR,7S,8aS) 2-phenyl-hexahydro-pyrano[3,2-d][1,3]dioxin-7-ol

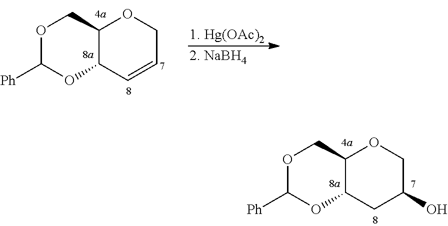

A solution of Hg(OAc)₂ (477 mg, 1.50 mmol) in 15.0 mL water was added to THF (8.0 mL). The resulting yellow solution was stirred for 15 min. A solution of (4aR,8aS) 2-phenyl-4,4a,6,8a-tetrahydro-pyrano[3,2-d][1,3]dioxine (326 mg, 1.50 mmol) in THF (7.0 mL) was added and the resulting mixture was stirred for 24 h. NaBH₄ (9.2 mL, 3.0 M solution in water) was added and the resulting mixture was stirred for 2 h. The reaction mixture was then diluted with EtOAc (50 mL×2), and washed with water and brine (30 mL), dried over Na₂SO₄, concentrated in vacuo and purified by flash chromatography with 50% EtOAc-hexane to yield (4aR,7S,8aS) 2-phenyl-hexahydro-pyrano[3,2-d][1,3]dioxin-7-ol.

Yield=328 mg, 93%; colorless solid; R_{f}=0.25 in 50% EtOAc-hexane. ¹H NMR (CDCl₃, 500 MHz): δ 7.43-7.50 (m, 2H), 7.30-7.38 (m, 3H), 5.58 (s, 1H), 4.27 (dd, J=10.5, 5.0 Hz, 1H), 4.09 (br s, 1H), 3.93-4.01 (m, 1H), 3.91 (d, J=12.5 Hz, 1H), 3.76 (dd, J=10.5, 10.5 Hz, 1H), 3.66 (d, J=12.5, 1H), 3.37 (ddd, J=10.0, 10.0, 5.0 Hz, 1H), 2.27 (dd, J=13.0, 2.0 Hz, 1H), 1.81 (ddd, J=13.0, 12.5, 3.0 Hz, 1H). ¹³C NMR (CDCl₃, 125 MHz): δ 137.43 (C), 129.05 (CH), 128.32 (2CH), 126.08 (2CH), 101.99 (CH), 74.34 (CH), 74.23 (CH), 72.56 (CH₂), 69.01 (CH₂), 67.03 (CH), 35.62 (CH₂). MS (m/z, relative intensity): 236 (M⁺, 8), 235 (5), 130 (27), 105 (18), 87 (100).

[α]_{D}²⁵=7.52 (c 15.7 CHCl₃).

### Example 41

### Preparation of (4aR,7S,8aS) toluene-4-sulfonic acid 2-phenyl-hexahydropyrano[3,2-d][1,3]dioxin-7-yl ester

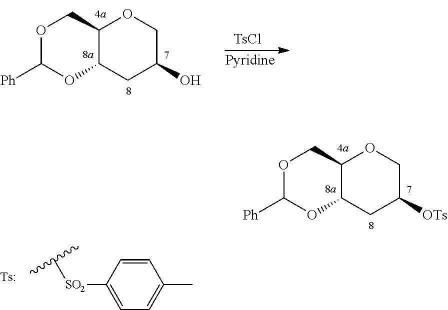

A solution of (4aR,7S,8aS) 2-phenyl-hexahydro-pyrano[3,2-d][1,3]dioxin-7-ol (118 mg, 0.5 mmol) and p-toluenesulfonyl chloride (100 mg, 0.5 mmol) in 2 mL of pyridine was stirred for 24 h at room temperature. The reaction mixture was evaporated, and the residue was dissolved in dichloromethane (50 mL). The solution was washed with water, saturated NaHCO₃ solution and brine (30 mL). The organic layer was dried over Na₂SO₄, concentrated in vacuo and purified by flash chromatography with 20% EtOAc-hexane to yield (4aR,7S,8aS) toluene-4-sulfonic acid 2-phenyl-hexahydropyrano[3,2-d][1,3]dioxin-7-yl ester.

Yield=187 mg, 96%; colorless solid; R_{f}=0.61 in 50% EtOAc-hexane. IR (neat): 3032, 2967, 2873, 1361, 1181, 1088, 920 cm⁻¹. ¹H NMR (CDCl₃, 500 MHz): δ 7.81 (d, J=8.0 Hz, 2H), 7.40-7.44 (m, 2H), 7.23-7.35 (m, 5H), 5.55 (s, 1H), 4.78 (s, 1H), 4.23 (dd, J=10.5, 5.0 Hz, 1H), 3.95 (d, J=13.0 Hz, 1H), 3.89-3.97 (m, 1H), 3.73 (dd, J=10.5, 10.5 Hz, 1H), 3.56 (d, J=13.0, 1H), 3.29-3.35 (m, 1H), 2.43 (s, 3H), 2.31 (d, J=13.5 Hz, 1H), 1.82 (ddd, J=13.5, 12.5, 2.5 Hz, 1H). ¹³C NMR (CDCl₃, 125 MHz): δ 145.03 (C), 137.25 (C), 133.72 (C), 129.97 (two of CH), 129.08 (CH), 128.28 (two of CH), 127.71 (two of CH), 126.04 (two of CH), 101.86 (CH), 76.71 (CH), 73.63 (CH), 73.37 (CH), 69.78 (CH₂), 68.78 (CH₂), 33.78 (CH₂), 21.63 (CH₃). MS (m/z, relative intensity): 390 (M⁺, 2), 389 (M⁺−1, 2), 284 (8), 173 (15), 155 (32), 105 (28), 91 (56), 69 (100).

### Example 42

### Preparation of (4aR,7R,8aS) 5-methyl-1-(2-phenyl-hexahydro-pyrano[3,2-d][1,3]dioxin-7-yl)-1H-pyrimidine-2,4-dione

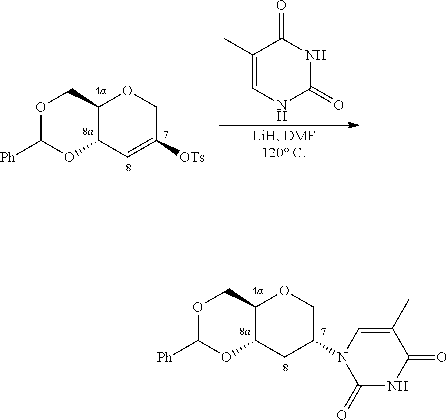

A mixture of Thymine (26 mg, 0.20 mmol) and lithium hydride (2 mg, 0.20 mmol) in 1.0 mL of dry DMF was stirred at 120□ for 1 h. After a solution of (4aR,7S,8aS) toluene-4-sulfonic acid 2-phenyl-hexahydropyrano[3,2-d][1,3]dioxin-7-yl ester (39 mg, 0.10 mmol) in 1.0 mL of dry DMF was added, stirring was continued for 3 h. The resulting mixture was cooled and diluted with EtOAc (30 mL×2), and washed with water and brine (30 mL), dried over Na₂SO₄, concentrated in vacuo and purified by flash chromatography with 10%-60% EtOAc-hexane to yield (4aR,7R,8aS) 5-methyl-1-(2-phenyl-hexahydro-pyrano[3,2-d][1,3]dioxin-7-yl)-1H-pyrimidine-2,4-dione.

Yield=6.9 mg, 20%; colorless liquid; R_{f}=0.40 in 70% EtOAc-hexane. IR (neat): 2932, 2857, 1677, 1579, 1128, 1000, 766, 706 cm⁻¹. ¹H NMR (CDCl₃, 500 MHz): δ 9.30 (br s, 1H), 7.42-7.49 (m, 2H), 7.30-7.38 (m, 3H), 7.01 (s, 1H), 5.55 (s, 1H), 4.75-4.84 (m, 1H), 4.25-4.32 (m, 1H), 3.98-4.05 (m, 1H), 3.61-3.79 (m, 2H), 3.44 (dd, J=11.0, 11.0 Hz, 1H), 3.25-3.39 (m, 1H), 2.30-2.40 (m, 1H), 1.90-2.10 (m, 1H), 1.91 (s, 3H). ¹³C NMR (CDCl₃, 125 MHz): δ 163.46 (C), 150.74 (C), 137.02 (C), 135.98 (CH), 129.22 (CH), 128.35 (CH), 128.29 (CH), 126.22 (CH), 126.08 (CH), 111.54 (C), 101.72 (CH), 76.54 (CH), 73.53 (CH), 68.94 (two of CH₂), 50.25 (CH), 33.44 (CH₂), 12.58 (CH₃). MS (m/z, relative intensity): 344 (M⁺, 6), 238 (17), 218 (8), 126 (97), 112 (88), 105 (97), 69 (100).

### Example 43

### Preparation of (3R,5S,6R) 1-(5-hydroxy-6-hydroxymethyl-tetrahydropyran-3-yl)-5-methyl-1H-pyrimidine-2,4-dione

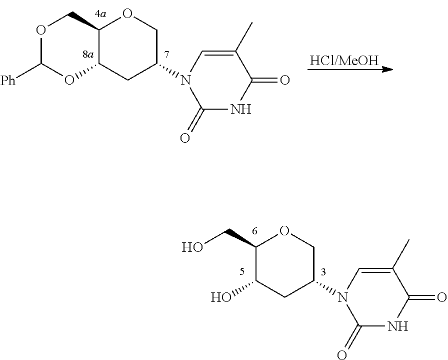

A solution of (4aR,7R,8aS) 5-methyl-1-(2-phenyl-hexahydro-pyrano[3,2-d][1,3]dioxin-7-yl)-1H-pyrimidine-2,4-dione (7.0 mg, 0.02 mmol) in 3.0 mL MeOH was added a solution of methanolic HCl (0.6 mL, prepared from 0.5 mL conc. HCl in 30 mL of MeOH). The resulting solution was stirred for 6 h at room temperature. The reaction mixture was evaporated in vacuo and purified by flash chromatography with 10% MeOH—CH₂Cl₂ to give (3R,5S,6R) 1-(5-hydroxy-6-hydroxymethyl-tetrahydropyran-3-yl)-5-methyl-1H-pyrimidine-2,4-dione.

Yield=4.0 mg, 77%; colorless liquid; R_{f}=0.44 in 15% MeOH—CH₂Cl₂. IR (neat) 3550-3100, 2925, 2850, 1737, 1459 cm⁻¹. ¹H NMR (d₆-DMSO, 500 MHz): δ 11.27 (br s 1H), 7.63 (s, 1H), 5.14 (br s, 1H), 4.55-4.58 (m, 1H), 4.14-4.20 (m, 1H), 3.60-3.79 (m, 3H), 3.38-3.44 (m, 2H), 2.96-3.05 (m, 1H), 2.00-2.10 (m, 1H), 1.70-1.90 (m, 1H). ¹³C NMR (d₆-DMSO, 125 MHz): δ 163.19 (C), 150.37 (C), 137.38 (CH), 108.48 (C), 82.21 (CH), 66.98 (CH₂), 64.53 (CH), 60.64 (CH₂), 49.28 (CH), 36.06 (CH₂), 11.54 (CH₃). MS (m/z, relative intensity) 256 (M⁺, 9), 238 (3), 195 (28), 152 (10), 127 (100).

### Example 44

### Preparation of (4aR,7R,8aS) 1-(2-phenyl-hexahydropyrano[3,2-d][1,3]dioxin-7-yl)-1H-pyrimidine-2,4-dione

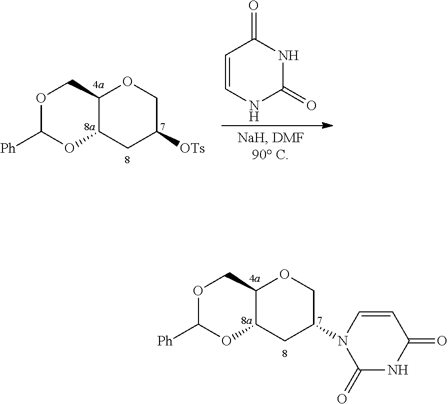

A mixture of Uracil (23 mg, 0.20 mmol) and sodium hydride (60% dispersion in mineral oil) (9 mg, 0.20 mmol) in 2.0 mL of dry DMF was stirred at 90° C. for 1 h. After a solution of (4aR,7S,8aS) toluene-4-sulfonic acid 2-phenyl-hexahydropyrano[3,2-d][1,3]dioxin-7-yl ester (39 mg, 0.10 mmol) in 1.0 mL of dry DMF was added, stirring was continued for 1 h. The resulting mixture was cooled and diluted with EtOAc (30 mL×2), and washed with water and brine (30 mL), dried over Na₂SO₄, concentrated in vacuo and purified by flash chromatography with 10%, 50% and 70% EtOAc-hexane to give (4aR,7R,8aS) 1-(2-phenyl-hexahydropyrano[3,2-d][1,3]dioxin-7-yl)-1H-pyrimidine-2,4-dione.

Yield=5.0 mg, 15%; colorless solid; R_{f}=0.28 in 70% EtOAc-hexane. IR (neat): 3180, 3059, 3022, 2925, 2859, 1699, 1105, 757 cm⁻¹. ¹H NMR (CDCl₃, 500 MHz): δ 8.76 (br s, 1H), 7.42-7.50 (m, 2H), 7.30-7.40 (m, 3H), 7.18 (d, J=8.0 Hz, 1H), 5.75 (d, J=7.0 Hz, 1H), 5.55 (s, 1H), 4.75-4.84 (m, 1H), 4.32 (dd, J=10.5, 5.0 Hz, 1H), 4.04 (dd, J=11.0, 5.0 Hz, 1H), 3.68-3.75 (m, 1H), 3.72 (dd, J=10.5, 10.5 Hz, 1H), 3.43 (dd, J=11.0, 11.0 Hz, 1H), 3.30-3.39 (m, 1H), 2.35-2.42 (m, 1H), 1.95-2.08 (m, 2H). ¹³C NMR (CDCl₃, 125 MHz): δ 162.37 (C), 150.43 (C), 140.10 (CH), 136.99 (C), 129.25 (CH), 128.38 (two of CH), 126.09 (two of CH), 103.05 (CH), 101.76 (CH), 76.51 (CH), 73.60 (CH), 68.98 (CH₂), 68.94 (CH₂), 50.56 (CH), 33.48 (CH₂). MS (m/z, relative intensity): 329 (M⁺−1, 2), 224 (5), 206 (5), 181 (22), 113 (100).

### Example 45

### Preparation of (3R,5S,6R) 1-(5-hydroxy-6-hydroxymethyl-tetrahydropyran-3-yl)-1H-pyrimidine-2,4-dione

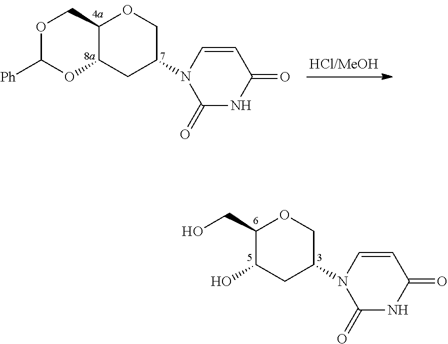

A solution of (4aR,7R,8aS) 1-(2-phenyl-hexahydropyrano[3,2-d][1,3]dioxin-7-yl)-1H-pyrimidine-2,4-dione (7.0 mg, 0.02 mmol) in 3.0 mL MeOH was added a solution of methanolic HCl (0.3 mL, prepared from 0.5 mL conc. HCl in 30 mL of MeOH). The resulting solution was stirred for 4 h at room temperature. The reaction mixture was evaporated in vacuo and purified by flash chromatography with 15% MeOH—CH₂Cl₂ to give (3R,5S,6R) 1-(5-hydroxy-6-hydroxymethyl-tetrahydropyran-3-yl)-1H-pyrimidine-2,4-dione

Yield=3.3 mg, 64%; colorless solid; R_{f}=0.29 in 15% MeOH—CH₂Cl₂. IR (neat): 3550-3100, 2930, 2856, 1729, 1451, 1105, 750, 698 cm⁻¹. ¹H NMR (DMSO-d₆, 500 MHz): δ 11.28 (br s 1H), 7.72 (d, J=8.0 Hz, 1H), 5.55 (d, J=8.5 Hz, 1H), 5.12 (d, J=5.0 Hz, 1H), 4.54 (t, J=6.0 Hz, 10H), 4.30-4.40 (m, 1H), 3.76 (dd, J=10.5, 3.0 Hz, 1H), 3.69 (dd, J=10.0, 6.0 Hz, 1H), 3.22-3.44 (m, 3H), 3.00-3.05 (m, 1H), 2.01-2.08 (m, 1H), 1.85 (dd, J=23.5, 11.5 Hz, 1H). ¹³C NMR (DMSO-d₆, 125 MHz): δ 162.54 (C), 150.38 (C), 141.83 (CH), 100.76 (CH), 82.15 (CH), 66.90 (CH₂), 64.51 (CH), 60.59 (CH₂), 49.70 (CH), 35.95 (CH₂). MS (m/z, relative intensity): 242 (M⁺, 5), 224 (10), 211 (5), 181 (39), 130 (22), 113 (100).

### Example 46

### Preparation of (4aR,7R,8aS) 1-(2-phenyl-hexahydropyrano[3,2-d][1,3]dioxin-7-yl)-5-iodo-1H-pyrimidine-2,4-dione

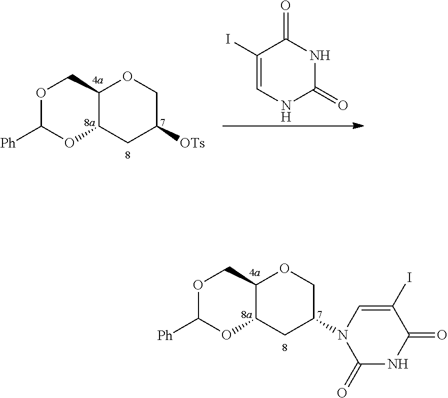

A mixture of 5-iodouracil (48 mg, 0.20 mmol) and sodium hydride (9 mg, 0.20 mmol) in 2.0 mL of dry DMF was stirred at 120□ for 1 h. A solution of (4aR,7S,8aS) toluene-4-sulfonic acid 2-phenyl-hexahydropyrano[3,2-d][1,3]dioxin-7-yl ester (39 mg, 0.10 mmol) in 1.0 mL of dry DMF was added, stirring was continued for 1 h. The resulting mixture was cooled and diluted with EtOAc (30 mL×2), and washed with water and brine (30 mL), dried over Na₂SO₄, concentrated in vacuo and purified by flash chromatography with 10%-50% EtOAc-hexane to give (4aR,7R,8aS) 1-(2-phenyl-hexahydropyrano[3,2-d][1,3]dioxin-7-yl)-5-iodo-1H-pyrimidine-2,4-dione.

Yield=5.0 mg, 11%; colorless solid; R_{f}=0.32 in 50% EtOAc-hexane. IR (neat): 3500-3100, 2919, 2844, 1735, 1468 cm⁻¹. ¹H NMR (CDCl₃, 500 MHz): δ 8.13 (s, 1H), 7.41-7.50 (m, 2H), 7.29-7.39 (m, 3H), 5.53 (s, 1H), 5.10-5.21 (m, 1H), 4.32 (dd, J=10.5, 4.5 Hz, 1H), 4.23 (dd, J=10.5, 5.0 Hz, 1H), 3.68 (dd, J=10.5, 10.5 Hz, 1H), 3.48-3.68 (m, 1H), 3.40 (dd, J=10.5, 10.5 Hz, 1H), 3.30-3.42 (m, 1H), 2.62-2.71 (m, 1H), 1.87 (dd, J=23.0, 11.5 Hz, 1H). ¹³C NMR (CDCl₃, 125 MHz): δ 161.26 (C), 160.54 (CH), 156.63 (C), 137.08 (C), 129.20 (CH), 128.37 (CH), 128.33 (CH), 126.19 (CH), 126.11 (CH), 101.78 (CH), 79.43 (C), 75.84 (CH), 73.41 (CH), 71.19 (CH), 69.03 (CH₂), 68.59 (CH₂), 34.55 (CH₂). MS (m/z, relative intensity): 456 (M⁺, 6), 349 (8), 331 (5), 306 (22), 238 (28), 105 (65), 69 (100).

### Example 47

### Preparation of (3R,5S,6R) 1-(5-hydroxy-6-hydroxymethyl-tetrahydropyran-3-yl)-5-iodo-1H-pyrimidine-2,4-dione

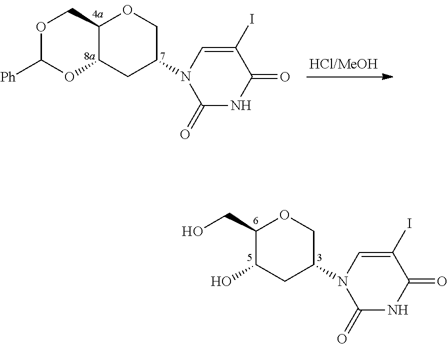

A solution of (4aR,7R,8aS) 1-(2-phenyl-hexahydropyrano[3,2-d][1,3]dioxin-7-yl)-5-iodo-1H-pyrimidine-2,4-dione (5.0 mg, 0.01 mmol) in 3.0 mL MeOH was added a solution of methanolic HCl (0.3 mL, prepared from 0.5 mL conc. HCl in 30 mL of MeOH). The resulting solution was stirred for 3 h at room temperature. The reaction mixture was evaporated in vacuo and purified by flash chromatography with 10% MeOH—CH₂Cl₂ to give (3R,5S,6R) 1-(5-hydroxy-6-hydroxymethyl-tetrahydropyran-3-yl)-5-iodo-1H-pyrimidine-2,4-dione.

Yield=1.8 mg, 45%; colorless liquid; R_{f}=0.25 in 10% MeOH—CH₂Cl₂. IR (neat) 3500-3100, 2919, 2844, 1735, 1468 cm⁻¹. ¹H NMR (d₆-DMSO, 500 MHz): δ 8.14 (s, 1H), 5.05 (d, J=6.0 Hz, 1H), 4.78-4.90 (m, 1H), 4.54 (dd, J=5.5, 6.0 Hz, 1H), 4.00-4.08 (m, 1H), 3.60-3.70 (m, 1H), 3.40-3.42 (m, 2H), 3.11 (dd, J=10.5, 10.5 Hz, 1H), 2.90-3.00 (m, 1H), 2.37-2.42 (m, 1H), 1.46 (dd, J=11.0, 22.5 Hz, 1H). MS (m/z, relative intensity): 368 (M⁺, 7), 238 (17), 195 (12), 130 (22), 69 (56), 41 (100).

### Example 48

### Preparation of the (4aR,7S,8S,8aR) epoxides

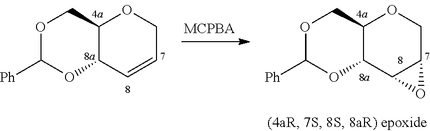

To a stirred solution of (4aR,8aS) 2-phenyl-4,4a,6,8a-tetrahydro-pyrano[3,2-d][1,3]dioxine (150 mg, 0.688 mmol) in CH₂Cl₂ (25 mL) was added meta-chloroperbenzoic acid (MCPBA) (75%, 236 mg, 1.37 mmol) at 0° C., and the mixture was stirred for 1 h at 0° C. After completion of the reaction, dimethyl sulfide (0.01 mL) was added and the resulting mixture was stirred for 10 min. The solvent was removed in vacuo and the residue was diluted with EtOAc (30 mL). The resulting solution was washed successively with saturated aqueous Na₂CO₃ (10 mL), water (10 mL×2) and brine (10 mL). The organic phase was dried over anhydrous Na₂SO₄. The solvent was removed in vacuo and the residue was purified by flash chromatography (10-20% EtOAc-hexane) to give the (4aR,7S,8S,8aR) epoxide.

Yield: 61 mg, 38% yield; R_{f}=0.54, 1:1 EtOAc-hexane. IR (neat): 2964, 2873, 1383, 1117, 1006, 751 cm⁻. ¹H NMR (CDCl₃, 400 MHz): δ 7.52-7.45 (m, 2H), 7.40-7.34 (m, 3H), 5.57 (s, 1H), 4.27 (dd, J=10.5, 4.7 Hz, 1H), 4.23 (d, 13.7 Hz, 1H), 3.95 (dd, J=13.6, 1.2 Hz, 1H), 3.70 (d, J=10.4 Hz, 1H), 3.66 (d, J=9.4 Hz, 1H), 3.49 (d, J=3.9 Hz, 1H), 3.23 (d, J=3.9 Hz, 1H), 3.18-3.08 (m, 1H). ¹³C NMR (CDCl₃, 100 MHz): δ 137.09 (C), 129.22 (CH), 128.35 (two of CH), 126.11 (two of CH), 102.37 (CH), 75.52 (CH), 69.46 (CH₂), 69.41 (CH), 65.38 (CH₂), 53.53 (CH), 49.97 (CH). [α]_{D}^{24.8}=+26.71 (0.14, CHCl₃)

### Example 49

### Preparation of (4aR,7R,8S,8aS) 1-(8-hydroxy-2-phenyl-hexahydropyrano[3,2-d][1,3]dioxin-7-yl)-5-methyl-1H-pyrimidine-2,4-dione

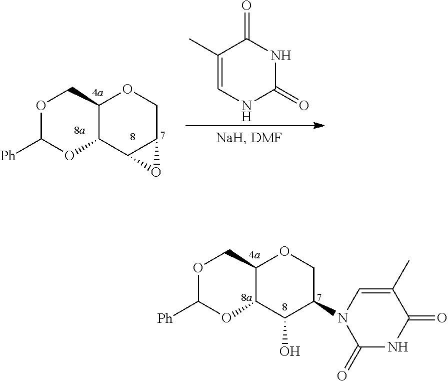

A two necked round bottom flask equipped with a magnetic stirrer and reflux condenser was charged with thymine (64 mg, 0.26 mmol), NaH (10.2 mg, 60% in oil, 0.25 mmol) and DMF (3 ml) under Argon atmosphere. The mixture was stirred at reflux for 2 hr. A solution of the (4aR,7S,8S,8aR) epoxide (20 mg, 0.085 mmol) in DMF (2 ml) was added and mixture was refluxed 24 hr. The reaction mixture was cooled to RT and diluted with 10% tert-BuOH-EtOAc 100 ml and quenched with brine (20 ml). The organic layer dried over Na₂SO₄ and the solvent was removed in vacuo. The crude residue was purified by column chromatography using 70% EtOAc-hexane to give (4aR,7R,8S,8aS) 1-(8-hydroxy-2-phenyl-hexahydropyrano[3,2-d][1,3]dioxin-7-yl)-5-methyl-1H-pyrimidine-2,4-dione.

Colorless solid, yield=19 mg, 47%; R_{f}=0.6 in 100% EtOAc. ¹H-NMR (CDCl₃, 500 MHz): δ 8.51 (s, 1H), 7.86 (s, 1H), 7.47-7.40 (m, 2H), 7.39-7.27 (m, 3H), 5.65 (s, 1H), 4.53-4.50 (m, 1H), 4.43-4.38 (m, 2H), 4.27 (br s, 1H), 4.14-4.09 (m, 1H), 4.06 (d, J=13.5 Hz, 1H), 3.77 (dd, J=10.0, 10.0 Hz, 1H), 3.70 (dd, J=10.0, 2.0 Hz, 1H). ¹³C-NMR (CDCl₃, 125 MHz): δ 163.51 (C), 150.94 (C), 137.86 (CH), 137.03 (C), 129.62 (CH), 128.63 (two of CH), 126.32 (two of CH), 111.50 (C), 102.44 (CH), 77.00 (CH), 69.14, (CH₂), 66.66 (CH) 66.53 (CH), 64.64 (CH₂), 56.23 (CH), 13.10 (CH₃).

### Example 50

### Preparation of (3R,4S,5S,6R) 1-(4,5-dihydroxy-6-hydroxymethyl-tetrahydropyran-3-yl)-5-methyl-1H-pyrimidine-2,4-dione

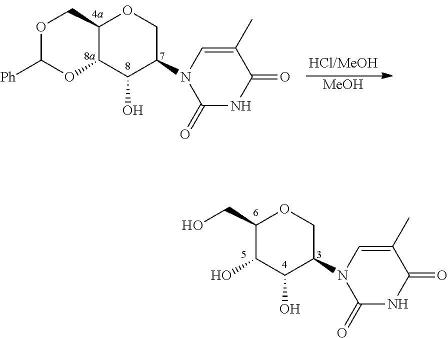

To a solution of (4aR,7R,8S,8aS) 1-(8-hydroxy-2-phenyl-hexahydropyrano[3,2-d][1,3]dioxin-7-yl)-5-methyl-1H-pyrimidine-2,4-dione (4 mg, 0.011 mmol) in MeOH (2 mL) was added a solution of methanolic HCl (0.3 mL, prepared from 0.5 mL conc. HCl in 30 mL of MeOH). The solution was stirred for 4 h at ambient temperature. The solution was diluted with ethyl acetate (40 mL), washed with NaHCO₃ (20 mL), dried over Na₂SO₄, concentrated in vacuo and purified by flash column chromatography with 20% MeOH—CH₂Cl₂ to give (3R,4S,5S,6R) 1-(4,5-dihydroxy-6-hydroxymethyl-tetrahydropyran-3-yl)-5-methyl-1H-pyrimidine-2,4-dione.

Yield=2 mg, 55%, white solid, R_{f}=0.29 in 20% MeOH—CH₂Cl₂. ¹H-NMR (CDCl₃, 500 MHz): δ 7.74 (s, 1H), 4.35 (dd, J=8.0, 3.5 Hz, 2H), 4.04-4.02 (m, 2H), 4.01-3.90 (m, 4H), 1.74 (s, 3H). ¹³C-NMR (CDCl₃, 125 MHz): δ 169.63 (C), 155.42 (C), 145.41 (CH), 114.09 (C), 79.69 (CH), 70.12 (CH), 68.34 (CH), 65.34 (CH₂), 62.96 (CH₂), 59.19 (CH), 14.62 (CH₃).

### Example 51

### Preparation of (4aR,7R,8S,8aS) 1-(8-hydroxy-2-phenyl-hexahydropyrano[3,2-d][1,3]dioxin-7-yl)-1H-pyrimidine-2,4-dione

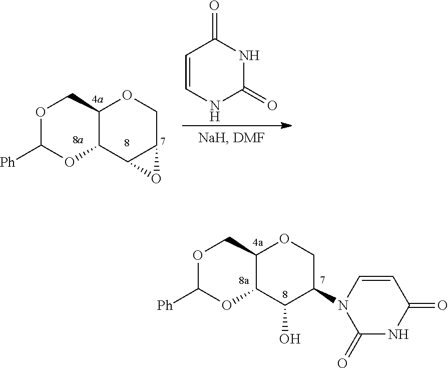

A two necked round bottom flask equipped with a magnetic stirrer and reflux condenser was charged with uracil (288 mg, 2.5 mmol), NaH (60 mg, 60% in oil, 2.5 mmol) and DMF (1.0 ml) under Argon atmosphere. The mixture was stirred at reflux for 1 hr. A solution of the (4aR,7S,8S,8aR) epoxide (100 mg, 0.43 mmol) in DMF (1.0 ml) was added and reflux was maintained for 24 hr. The reaction mixture was cooled to ambient temperature and diluted with 10% t-BuOH-EtOAc (200 ml) and quenched with brine (20 ml). The organic layer dried over Na₂SO₄ and the solvent was removed in vacuo. The crude residue was purified by column chromatography using 50% EtOAc-hexane to give (4aR,7R,8S,8aS) 1-(8-hydroxy-2-phenyl-hexahydropyrano[3,2-d][1,3]dioxin-7-yl)-1H-pyrimidine-2,4-dione.

Colorless solid; Yield=43 mg, 29%; R_{f}=0.39 in 100% EtOAc. ¹H-NMR (CDCl₃, 500 MHz): δ 9.52 (s, 1H), 7.97 (d, J=8.0 Hz, 1H), 7.38-7.44 (m, 2H), 7.29-7.38 (m, 3H), 5.74 (d, J=8.0 Hz, 1H), 5.58 (s, 1H), 4.45 (br s, 1H), 4.20-4.40 (m, 3H), 4.02-4.10 (m, 1H), 3.92 (d, J=14.0 Hz, 1H), 3.69 (dd, J=10.0, 10.0 Hz, 2H), 3.62 (d, J=10.0 Hz, 1H), 3.44 (br. s, 1-OH).

¹³C-NMR (CDCl₃, 125 MHz): δ 163.08 (C), 151.04 (C), 142.08 (CH), 136.86 (C), 129.28 (CH), 128.30 (two of CH), 126.14 (two of CH), 102.85 (CH), 102.22 (CH), 76.63 (CH), 68.85 (CH₂), 66.33 (CH) 65.98 (CH), 64.19 (CH₂), 56.45 (CH).

### Example 52

### Preparation of (3R,4S,5S,6R) 1-(4,5-dihydroxy-6-hydroxymethyl-tetrahydropyran-3-yl)-1H-pyrimidine-2,4-dione

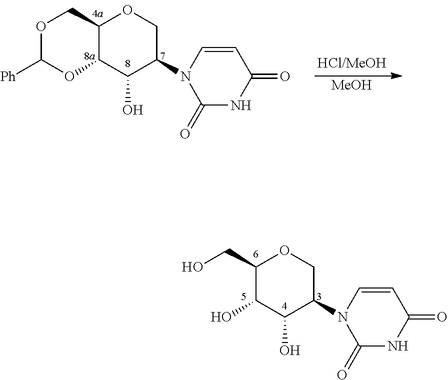

To a solution of (4aR,7R,8S,8aS) 1-(8-hydroxy-2-phenyl-hexahydropyrano[3,2-d][1,3]dioxin-7-yl)-1H-pyrimidine-2,4-dione (10 mg, 0.029 mmol) in MeOH (5 mL) was added a solution of methanolic HCl (0.3 mL, prepared from 0.5 mL conc. HCl in 30 mL of MeOH). The solution was stirred for 2 h at ambient temperature. The solution was diluted with ethyl acetate (40 mL), washed with NaHCO₃ (20 mL), dried over Na₂SO₄, concentrated in vacuo and purified by flash column chromatography with 20% MeOH—CH₂Cl₂ to give (3R,4S,5S,6R) 1-(4,5-dihydroxy-6-hydroxymethyl-tetrahydropyran-3-yl)-1H-pyrimidine-2,4-dione.

Yield=6.7 mg, 89%, white solid, R_{f}=0.65 in 50% MeOH—CH₂Cl₂. ¹H-NMR (D₂O, 500 MHz): δ 7.94 (d, J=8.0 Hz, 1H), 5.69 (d, J=8.0 Hz, 1H), 4.35-4.30 (m, 1H), 4.05-3.90 (m, 3H), 3.71-3.60 (m, 4H). ¹³C-NMR (D₂O, 125 MHz): δ 166.58 (C), 152.47 (C), 144.76 (CH), 101.95 (CH), 76.62 (CH), 67.16 (CH), 65.25 (CH), 62.53 (CH₂), 60.38 (CH₂), 56.64 (CH).

### Example 53

### Preparation of (4aR,7R,8S,8aS) 1-(8-hydroxy-2-phenyl-hexahydropyrano[3,2-d][1,3]dioxin-7-yl)-5-iodo-1H-pyrimidine-2,4-dione

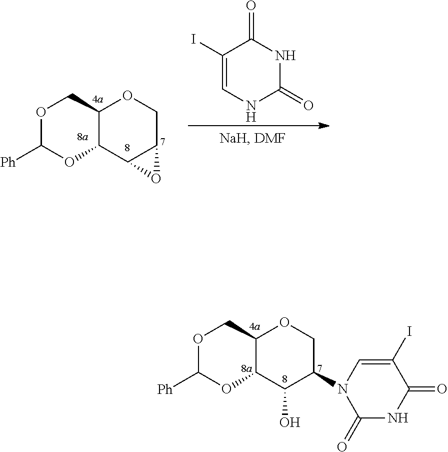

A two necked round bottom flask equipped with a magnetic stirrer and reflux condenser was charged with 5-iodouracil (81 mg, 0.34 mmol), NaH (13.6 mg, 60% in oil, 0.34 mmol) and DMF (3 ml) under Argon atmosphere. The mixture was stirred at reflux for 2 hr. A solution of the (4aR,7S,8S,8aR) epoxide (20 mg, 0.085 mmol) in DMF (2 ml) was added and mixture was refluxed 24 hr. The reaction mixture was cooled to RT and diluted with 10% t-BuOH/EtOAc (100 ml) and quenched with brine (20 ml). The organic layer dried over Na₂SO₄ and the solvent was removed in vacuo. The crude residue was purified by column chromatography using 70% EtOAc-hexane to give (4aR,7R,8S,8aS) 1-(8-hydroxy-2-phenyl-hexahydropyrano[3,2-d][1,3]dioxin-7-yl)-5-iodo-1H-pyrimidine-2,4-dione.

Colorless solid, yield=8 mg, 20%; Rf=0.36 in 100% EtOAc. ¹H-1-NMR (CD₃OD, 500 MHz): δ 9.09 (br s, 1H), 8.41 (s, 1H), 7.40-7.45 (m, 2H), 7.30-7.35 (m, 3H), 5.53 (s, 1H), 4.45 (br s, 1H), 4.26-4.40 (m, 2H), 4.26 (br s, 1H), 4.05-4.10 (m, 2H), 4.00 (d, J=14.0 Hz, 1H), 3.74 (dd, J=10.5, 10.5 Hz, 1H), 3.68 (d, J=9.5 Hz, 1H). ¹³C-NMR (CDCl₃, 125 MHz): δ 159.68 (C), 150.52 (C), 146.54 (CH), 136.76 (C), 129.36 (CH), 128.37 (two of CH), 126.24 (C), 126.13 (two of CH), 102.22 (CH), 76.45 (CH), 68.85 (CH₂), 66.50 (CH), 65.87 (CH), 64.16 (CH₂), 56.64 (CH).

### Example 54

### Preparation of (3R,4S,5S,6R) 1-(4,5-dihydroxy-6-hydroxymethyl-tetrahydropyran-3-yl)-5-iodo-1H-pyrimidine-2,4-dione

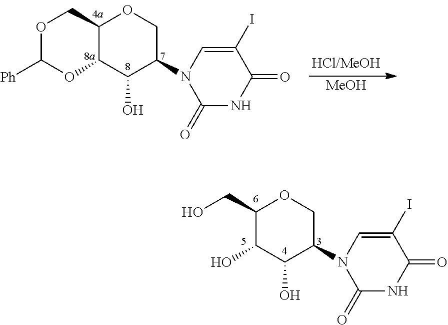

To a solution of (4aR,7R,8S,8aS) 1-(8-hydroxy-2-phenyl-hexahydropyrano[3,2-d][1,3]dioxin-7-yl)-5-iodo-1H-pyrimidine-2,4-dione (8 mg, 0.017 mmol) in MeOH (2 mL) was added a solution of methanolic HCl (0.3 mL, prepared from 0.5 mL conc. HCl in 30 mL of MeOH). The solution was stirred for 4 h at ambient temperature. The solution was diluted with ethyl acetate (40 mL), washed with NaHCO₃ (20 mL), dried over Na₂SO₄, concentrated in vacuo and purified by flash column chromatography with 20% MeOH—CH₂Cl₂ to give (3R,4S,5S,6R) 1-(4,5-dihydroxy-6-hydroxymethyl-tetrahydropyran-3-yl)-5-iodo-1H-pyrimidine-2,4-dione.

Yield=5 mg, 77%, white solid, R_{f}=0.54 in 20% MeOH—CH₂Cl₂. ¹H-NMR (D₂O, 500 MHz): δ 8.34 (s, 1H), 5.31 (s, 1H), 4.36 (dd, J=10.0, 5.0 Hz, 2H), 3.92-4.05 (m, 4H), 3.67-3.71 (m, 3H), 3.21 (br s, 1H).

### Example 55

### Preparation of (4aR,7R,8S,8aR) methanesulfonic acid 7-(2,4-dioxo-3,4-dihydro-2H-pyrimidin-1-yl)-2-phenyl-hexahydro-pyrano[3,2-d][1,3]dioxin-8-yl ester

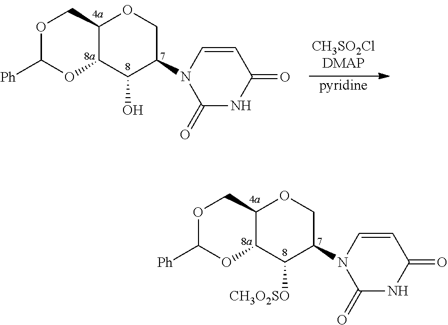

To a stirred solution of (4aR,7R,8S,8aS) 1-(8-hydroxy-2-phenyl-hexahydropyrano[3,2-d][1,3]dioxin-7-yl)-1H-pyrimidine-2,4-dione (15 mg, 0.04 mmol) in pyridine (1 ml) was added DMAP (33 mg, 0.24 mmol) and CH₃SO₂Cl (20 mg, 0.16 mmol). The mixture was stirred for 16 hr, diluted with 10% tert-BuOH-EtOAc (100 ml), washed with water (10 ml×2) and brine (10 ml). The organic layer dried over Na₂SO₄, the solvent was removed in vacuo and the residue was purified by column chromatography using 70% EtOAc-hexane to give (4aR,7R,8S,8aR) methanesulfonic acid 7-(2,4-dioxo-3,4-dihydro-2H-pyrimidin-1-yl)-2-phenyl-hexahydro-pyrano[3,2-d][1,3]dioxin-8-yl ester.

Yield=19 mg, 47%. (CDCl₃, 500 MHz): δ 8.47 (br s, 1H), 7.98 (d, J=8.0 Hz, 1H), 7.42-7.39 (m, 2H), 7.38-7.30 (m, 3H), 5.82 (dd, J=8.0, 2.0 Hz, 1H), 5.62 (br s, 1H), 5.19 (br s, 1H), 4.68 (m, 1H), 4.44-4.36 (m, 2H), 4.18 (d, J=14.0 Hz, 1H), 4.09-4.00 (m, 1H), 3.80-3.70 (m, 2H), 3.08 (s, 3H). ¹³C-NMR (CDCl₃, 125 MHz): δ 162.39 (C), 150.52 (C), 141.37 (CH), 136.63 (C), 129.68 (CH), 128.67 (two of CH), 126.16 (two of CH), 103.65 (CH), 102.59 (CH), 74.66 (CH), 73.90 (CH), 68.90 (CH₂), 67.33 (CH), 64.54 (CH₂), 55.61 (CH), 39.11 (CH₃).

### Example 56

### Preparation of (4aR,7R,8R,8aS) 1-(8-hydroxy-2-phenyl-hexahydro-pyrano[3,2-d]-[1,3]dioxin-7-yl)-1H-pyrimidine-2,4 dione

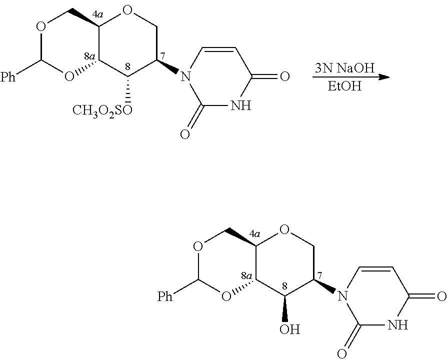

To a stirred solution of (4aR,7R,8S,8aR) methanesulfonic acid 7-(2,4-dioxo-3,4-dihydro-2H-pyrimidin-1-yl)-2-phenyl-hexahydro-pyrano[3,2-d][1,3]dioxin-8-yl ester (7 mg, 0.016 mmol) in 0.5 ml EtOH and was added 3N aqueous NaOH (1.5 ml), the mixture was stirred for 2 hr. After completion of the reaction, diluted with 10% t-BuOH-EtOAc 100 ml, and washed with water (10 ml). The organic layer dried over Na₂SO₄ and the solvent was removed in vacuo and the residue was purified by column chromatography using 70% EtOAc-hexane to give (4aR,7R,8R,8aS) 1-(8-hydroxy-2-phenyl-hexahydro-pyrano[3,2-d]-[1,3]dioxin-7-yl)-1H-pyrimidine-2,4 dione. ¹H-NMR (CDCl₃, 500 MHz): δ 8.77 (s, 1H), 8.02 (d, J=7.5 Hz, 1H), 7.44-7.28 (m, 5H), 5.79 (d, J=7.5 Hz, 1H), 5.60 (s, 1H), 4.49 (br s, 1H), 4.40-4.30 (m, 2H), 4.24 (br s, 1H), 4.10-4.00 (m, 2H), 3.75-3.60 (m, 2H), 2.93 (br s, 1H). ¹³C-NMR (CDCl₃, 125 MHz): δ 162.37 (C), 150.67 (C), 142.03 (CH), 136.74 (C), 129.37 (CH), 128.37 (2CH), 126.07 (2CH), 102.77 (CH), 102.24 (CH), 76.67 (CH), 68.82 (CH₂), 66.44 (CH), 66.15 (CH), 64.36 (CH₂), 56.08 (CH).

### Example 57

### Preparation of (3R,4R,5S,6R) 1-(4,5-dihydroxy-6-hydroxymethyl-tetrahydropyran-3-yl)-1H-pyrimidine-2,4-dione

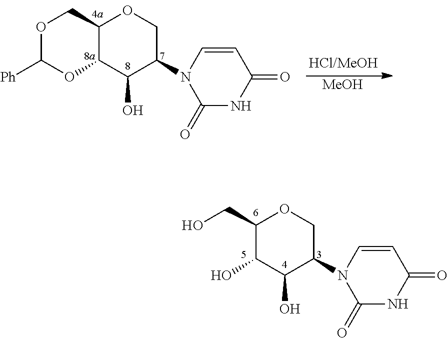

To a solution of (4aR,7R,8R,8aS) 1-(8-hydroxy-2-phenyl-hexahydro-pyrano[3,2-d]-[1,3]dioxin-7-yl)-1H-pyrimidine-2,4 dione (5 mg, 0.014 mmol) in MeOH (2 mL) was added a solution of methanolic HCl (0.3 mL, prepared from 0.5 mL conc. HCl in 30 mL of MeOH). The solution was stirred for 4 h at ambient temperature. The solution was diluted with ethyl acetate (40 mL), washed with NaHCO₃ (20 mL), dried over Na₂SO₄, concentrated in vacuo and purified by flash column chromatography with 20% MeOH—CH₂Cl₂ to give (3R,4R,5S,6R)-1-(4,5-dihydroxy-6-hydroxymethyl-tetrahydropyran-3-yl)-1H-pyrimidine-2,4-dione.

Yield=1 mg, 28%, white solid, R_{f}=0.31 in 20% MeOH—CH₂Cl₂. ¹H-NMR (CDCl₃, 500 MHz): δ 8.16 (d, J=8.5 Hz, 1H), 5.74 (d, J=8.5 Hz, 1H), 4.15 (d, J=14.0 Hz, 1H), 3.99-3.92 (m, 2H), 3.82-3.75 (m, 1H), 3.73-3.67 (m, 2H), 3.55 (dd, J=10.0, 10.0 Hz, 1H), 3.31-3.39 (m, 1H), 3.08-3.02 (m, 1H).

Although the invention has been described with reference to the above examples, it will be understood that modifications and variations are encompassed within the spirit and scope of the invention. Accordingly, the invention is limited only by the following claims.

What is claimed is:

1. A compound having the structure A: 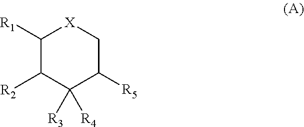
wherein:
a) X is a moiety selected from the group consisting of oxygen, sulfur, and —NR₆;
b) R₁ is a substitutent selected from the group consisting of C₁₋₁₀ substituted alkyl, and —CH₂OR₁₁;
c) R₂ is a substitutent selected from the group consisting of hydrogen, halogen, —OR₁₂, —SR₁₂, and —NHR₁₂;
d) each of R₃ and R₄ is a substitutent independently selected from the group consisting of hydrogen, halogen, azido, —CN, C₁₋₁₀ alkylcarboxy, C₁₋₁₀ arylcarboxy, and —OSO₂R₇, with the further proviso that R₃ and R₄ cannot both be hydrogen;
e) R₅ is a substitutent selected from the group consisting of heteroaryl, saturated heterocyclic, and —NR₈R₉;
f) R₆ is a substitutent selected from the group consisting of hydrogen, amino protecting group, C₁₋₁₀ alkyl, C₁₋₁₀ substituted alkyl, aryl, C₁₋₁₀ alkylcarbonyl, arylcarbonyl, C₁₋₁₀ alkyloxycarbonyl, aryloxycarbonyl, and —SO₂R₁₀;
g) each of R₈ and R₉ is a substitutent independently selected from the group consisting of hydrogen, and C₁₋₁₀ alkyl, or R₈, R₉ and the nitrogen atom to which R₈ and R₉ are attached, combine to form a saturated heterocyclic or heteroaryl ring;
h) each of R₇ and R₁₀ is a substitutent independently selected from the group consisting of C₁₋₁₀ alkyl, C₁₋₁₀ substituted alkyl, aryl and substituted aryl;
i) R₁₁ is a substitutent selected from the group consisting of hydrogen, hydroxyl protecting group, —P(O)(OR₁₅)(OR₁₆), and —CH₂P(O)(OR₁₅)(OR₁₆);
j) R₁₂ is a substitutent selected from the group consisting of hydrogen, —PR₁₃R₁₄, hydroxyl protecting group if R₂ is —OR₁₂, thiol protecting group if R₂ is —SR₁₂, and amino protecting group if R₂ is —NHR₁₂;
or if R₁ is —CH₂OR₁₁ and R₂ is —OR₁₂, then R₁₁, R₁₂ and the oxygen atoms to which R₁₁ and R₁₂ are attached, combine to form a cyclic acetal or ketal;
k) each of R₁₃ and R₁₄ is a substitutent independently selected from the group consisting of —NR₈R₉, and —OCH₂CH₂CN; and
1) each of R₁₅ and R₁₆ is a substitutent independently selected from the group consisting of hydrogen, and C₁₋₁₀ alkyl,
or a pharmaceutically acceptable salt thereof.

2. Compounds selected from a group having the formulae: 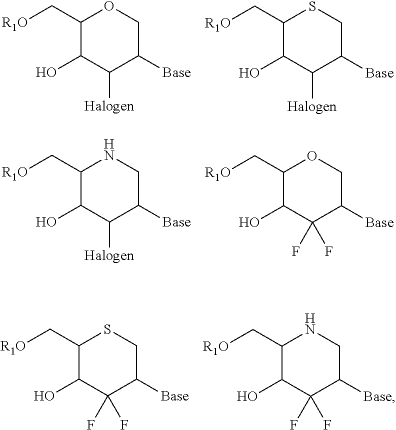
wherein:
a) R₁ is a substitutent selected from a group consisting of —H, PO₃H, and —CH₂OPO₃H;
b) halogen is selected from F, Cl, Br, and I; and
c) base is a moiety selected from the group having the formulae: 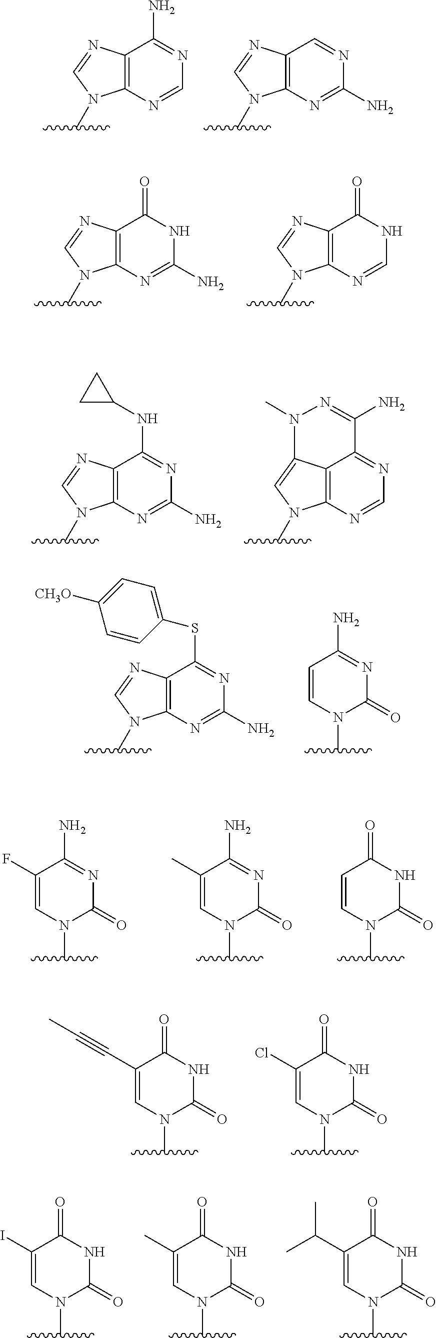 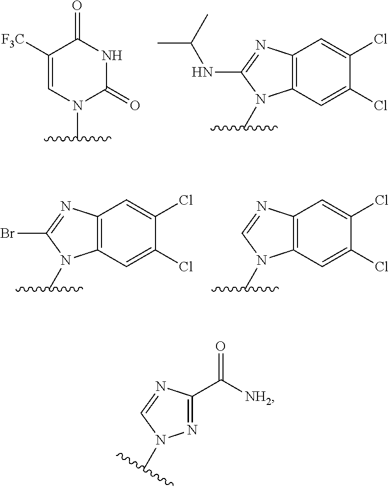
and pharmaceutically acceptable salts thereof.

3. A pharmaceutical composition comprising at least one of the compounds of claim 1, and pharmaceutically acceptable pro-drugs and salts thereof.

4. The pharmaceutical composition of claim 3, further including a pharmaceutically acceptable vehicle, for enteral, parenteral, topical or ocular administration.

5. The pharmaceutical composition of claim 3, for the treatment or prophylaxis of viral infections.

6. The pharmaceutical composition of claim 3, for the treatment or prophylaxis of bacterial infections.

7. The pharmaceutical composition of claim 3, for the treatment or prophylaxis of fungal infections.

8. The pharmaceutical composition of claim 3, for use in antisense therapy.

9. The pharmaceutical composition of claim 3, for the treatment or prophylaxis of cancer, diabetes and other diseases of genetic origin.

10. The pharmaceutical composition of claim 8, for the treatment or prophylaxis of cancer, diabetes and other diseases of genetic origin.

11. A method for treating cancer, the method comprising administering to a subject in need thereof an effective amount of at least one compound of claim 1, in a pharmaceutically acceptable vehicle.

12. A method for treating cancer, the method comprising administering to a subject in need thereof an effective amount of at least one compound of claim 1, in a pharmaceutically acceptable vehicle.

13. The method of claim 12, wherein cancer is selected from a group consisting of mammary cancer, prostate cancer, kidney cancer, Karposi's sarcoma, colon cancer, cervical cancer, lung cancer, cutaneous T-cell lymphoma, cancer of the head and neck, cancers of the aerodigestive pathway, skin cancer, bladder cancer, sarcomas, leukoplakias, and acute promyelocytic leukemia.

14. The method of claim 12, further comprising administering, in combination with a compound of claim 1, at least one other chemotherapeutic agent selected from the group consisting of Busulfan, Carboplatin, Cisplatin, Cyclophosphamide, Cytosine arabinoside, Etoposide, 5-Fluorouracil, Melphalan, Methotrexate, Mitoxantrone, Taxol, Interferon, Fareston, Arzoxifene, Evista, and Tamoxifen.

15. A method for modulating the expression of enzymes, proteins, nuclear factors or receptors in cells or tissues comprising contacting the cells or tissues with at least one compound of claim 1 or 2.

16. A method for modulating the expression of enzymes, proteins, nuclear factors or receptors in cells or tissues comprising contacting the cells or tissues with at least one composition of any one of claims 3-10.

17. A method for treating a subject suspected of having or being prone to a disease or condition associated with expression of said enzymes, proteins, nuclear factors or receptors, the method comprising administering to a subject in need thereof an effective amount of at least one compound of claim 1, in a pharmaceutically acceptable vehicle.

18. A nucleic acid probe constructed from at least one compound of claim 1.

19. A method for using a nucleic acid probe according to claim 18 for the identification and quantification of a bacterium, virus or any other organism in sputum, urine, blood, tissue sections, food, soil, water.
